(19) United States
(12) Patent Application Publication
(10) Pub. No.: US 2025/0122244 A1
(43) Pub. Date: Apr. 17, 2025

(54) METHOD FOR PRODUCING AN ANTITUMORAL ARENAVIRUS AS WELL AS ARENAVIRUS MUTANTS

(71) Applicant: ABALOS THERAPEUTICS GMBH, Dusseldorf (DE)

(72) Inventors: Karl LANG, Essen (DE); Cornelia HARDT, Bochum (DE); Michael BERGERHAUSEN, Duisburg (DE); Hilal BHAT, Essen (DE)

(73) Assignee: ABALOS THERAPEUTICS GMBH, Dusseldorf (DE)

(21) Appl. No.: 18/927,732

(22) Filed: Oct. 25, 2024

Related U.S. Application Data
(63) Continuation of application No. 17/275,967, filed on Mar. 12, 2021, now Pat. No. 12,129,281.

(30) Foreign Application Priority Data
Sep. 12, 2018 (DE) 10 2018 215 551.8

(51) Int. Cl. C07K 14/005; A61K 35/768; A61K 45/06; A61P 35/00; C12N 7/00

(52) U.S. Cl. CPC C07K 14/005; A61K 35/768; A61K 45/06; A61P 35/00; C12N 7/00; C12N 2760/10021; C12N 2760/10022; C12N 2760/10032; C12N 2760/10052; C12N 2760/10071

(57) ABSTRACT

The invention relates to methods of treating or preventing a tumor.

### CROSS REFERENCE TO RELATED APPLICATIONS

The present application is a continuation application of U.S. patent application Ser. No. 17/275,967, now U.S. Pat. No. 12,129,281, which is the United States national stage patent application of International Application No. PCT/EP2019/074307, filed Sep. 12, 2019, which claims the right of priority of German Patent Application DE 10 2018 215 551.8 filed on Sep. 12, 2018, the content of each of which is hereby incorporated by reference in its entirety for all purposes.

### SEQUENCE LISTING

The application contains a Sequence Listing which has been submitted electronically in .XML format and is hereby incorporated by reference in its entirety. Said .XML copy, created on Oct. 25, 2024, is named “SCH-6900-CON1.xml” and is 195 kilobytes in size. The sequence listing contained in this .XML file is part of the specification and is hereby incorporated by reference herein in its entirety.

### FIELD OF THE INVENTION AND BACKGROUND

The invention relates to a method for the preparation of an antitumoral arenavirus, arenavirus mutants, isolated proteins or peptides, in particular isolated glycoproteins, an arenavirus and nucleic acids encoding for corresponding proteins or peptides.

Arenaviruses belong to the family of human pathogenic pleomorphic RNA viruses. Diseases with these viruses belong to the zoonoses due to their natural reservoir in animals, predominantly rodents. Zoonoses are diseases that can be transmitted from animals to humans and vice versa from humans to animals.

At least eight arenaviruses are known to cause a disease in humans, typically aseptic meningitis and haemorrhagic fever. Known viruses that can cause a disease in humans are the lymphocytic choriomeningitis virus (LCMV), guanarito virus (GTOV), junin virus (JUNV), lassa virus (LASV), lujo virus (LUJV), machupo virus (MACV), sabia virus (SABV) and whitewater arroyo virus (WWAV).

Arenaviruses with the genus mammarenavirus are divided into two groups, the Old World Arenaviruses and the New World Arenaviruses. These groups differ geographically and genetically. Old World Arenaviruses, such as the lymphocytic choriomeningitis virus, were found mainly in countries of the Eastern Hemisphere, such as European, Asian and African countries. In contrast, New World Arenaviruses have been found in Western Hemisphere countries such as Argentina, Bolivia, Venezuela, Brazil and the United States of America.

Arenaviruses replicate in the cell and enter the extracellular space as virions (infectious particles). Virions have a pleomorphic, often round shape with a diameter of mostly 110 nm to 130 nm up to 50 nm to 300 nm. The capsid of the virion is surrounded by an envelope protein consisting of a double lipid membrane and homotrimers of glycoproteins (GP1 and GP2) that protrude in the shape of spikes. GP1 is directed outwards and binds to the cellular receptor during infection. GP2 is directed in the opposite direction and mediates fusion with the cell. The Z-proteins lay like a ring below the lipid layer, located inside the nucleocapsid the ribonucleoprotein (RNP)-complexes each consists of the shorter RNA segment (S segment 3.5 kb) respectively the longer RNA segment (L segment 7.2 kb) and the nucleoproteins. The L-protein, the viral polymerase, is associated with the RNP complexes. L- and S-segments carry the genetic information and code for two proteins each. The single-stranded RNAs have a mixed (i.e. ambisense polarity), at their 3′ untranslated ends the sequences (approx. 19-30 bp) are conserved, also within the virus family. First, the mRNAs of nucleoprotein and L-protein are transcribed, followed by replication and mRNA transcription of Z-protein and the glycoprotein precursor, and finally followed by translation and modification of the viral proteins.

The use of arenaviruses as vaccination vectors is well known. A prominent example is the vaccination virus Candid #1 used against Argentine haemorrhagic fever. This is a vaccination variant of the Junin virus.

From WO 2009/083210 A1, the use of replication defective, i.e. genetically modified arenavirus particles (virions) inter alia for the treatment of neoplastic diseases such as melanoma, prostate carcinoma, breast carcinoma and lung carcinoma is known. The publication “Development of replication-defective lymphocytic choriomeningitis virus vectors for the induction of potent CD8+ T cell immunity” (Nature Medicine, vol. 16, no. 3, March 2010, p. 339-345; doi: 10.1038/nm.2104) mentions as a potential application area for such virus particles cancer immunotherapy.

Further from WO 2006/008074 A1 the use of packaging cells which produce both retroviral and with arenavirus-glycoprotein pseudotyped virions for gene therapy of solid tumors is known.

The above described state-of-the-art methods for the treatment of tumors are based on the use of virus particles that are very complicated to generate by means of genetic engineering. In addition, in case of gene therapy treatment methods a sufficient, therapeutically effective transduction of the tumor tissue with genetically engineered virions or packaging cells which produce virions is often not achievable.

From WO 2016/166285 A1, however, arenaviruses for the treatment and/or prevention of tumors are known, whereat said arenaviruses are free of genomic foreign RNA. Additionally, from WO 2016/166285 A1 a method for the production of arenaviruses with tumor regressive properties is known.

Nevertheless, there is still a need for specific and in particular therapeutically effective arenaviruses for use in the treatment and/or prevention of tumors.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows LCMV spread in MC57, C643, H1975 and UKE-Mel-13a cells. Ø=without infection, LCMV=LCMV infected.

FIG. 2 shows the number of infectious virus particles measured in the supernatant after 24 hours of infection in MC57 cells, Tramp-C2 cells, and primary tumor cell cultures 511950 and 511950R.

FIG. 3 shows an exemplary passaging protocol for LCMV-WE in UKE-Mel-13a cell cultures.

FIG. 4 shows an exemplary passaging protocol for LCMV-WE in H1975 cell cultures.

FIG. 5 shows an exemplary passaging protocol for LCMV-WE in H1975 cell cultures.

FIG. 6 shows the number of infectious virus particles measured in the supernatant after 12 hours of infection with LCMV-P52 and LCMV-WE in H1975 cells.

FIG. 7 shows the number of infectious virus particles measured in the supernatant after 24 hours of infection with LCMV-P52 and LCMV-WE in murine bone marrow-derived dendritic cells.

FIG. 8 shows LCMV-P52 and LCMV WE spread in H1975 cells.

FIG. 9 shows LCMV-P52 and LCMV WE spread in UKE-Mel-13a cells

FIG. 10 shows activation of the innate immune system as determined by measuring IFN-alpha in serum from C57BL/6 mice following infection with LCMV-WE or LCMV-P52.

FIG. 11 shows a time course of activation of the adaptive immune system as determined by measuring virus-specific CD8+ T cells in C57BL/6 mice following infection with LCMV-WE or LCMV-P52.

FIG. 12 shows a time course of MOPC tumor growth in NOD.SCID mice following infection with LCMV-WE or LCMV-P52.

FIG. 13 shows a time course of MOPC tumor growth in C57BL/6 mice following infection with LCMV-P52.

FIG. 14 shows a time course of CMT167 tumor growth in C57BL/6 mice following infection with LCMV-P52.

FIG. 15 shows CMT167 tumor growth in lung tissue of control C57BL/6 mice and mice infected with LCMV-P52.

FIG. 16 shows measurement of CD8+ T cells in the blood of control OY-1 mice and mice infected with LCMV-P52 following administration of B16F10-OVA cells.

FIG. 17 shows measurement of CD8+ T cells in the spleen of control OY-1 mice and mice infected with LCMV-P52 following administration of B16F10-OVA cells.

FIG. 18 shows a time course of B16F10-OVA tumor growth in C57BL/6 mice following infection with LCMV-WE, treatment with LCL-161, or co-treatment with both.

FIG. 19 shows a time course of B16F10-OVA tumor growth in C57BL/6 mice following infection with LCMV-WE, treatment with LCL-161, or co-treatment with both.

FIG. 20 shows a time course of B16F10-OVA tumor growth in C57BL/6 mice following infection with LCMV-P52, treatment with anti-PD-1, or co-treatment with both.

FIG. 21 shows the number of infectious virus particles measured in the supernatant after 24 hours of infection of H1975 cells with LCMV-WE, LCMV-P52.1 (I181M, R185W), LCMV-P52-1.3 (R185W) and LCMV-P52-2.1 (I181M).

FIG. 22 shows the number of infectious virus particles measured in the supernatant after 24 hours of infection of HCC1954 cells with LCMV-WE, LCMV-P52.1 (I181M, R185W), LCMV-P52-1.3 (R185W) and LCMV-P52-2.1 (I181M).

FIG. 23 shows the number of infectious virus particles measured in the supernatant after 24 hours of infection of Murine Pancreatic cancer (511950) cells, human Melanoma (UKE118b) cells, or human Melanoma (UKE118c) cells with LCMV-WE or LCMV-42 (I181M).

FIG. 24 shows measurement of LCMV-GP33-specific CD8+ T cells in the blood of C57BL/6 mice infected with LCMV-WE, LCMV-P52.1 (I181M, R185W), LCMV-P52-1.3 (R185W) and LCMV-P52-2.1 (I181M).

FIGS. 25A-E show nucleic acid and amino acid sequences of LCMV-WE (FIG. 25A), LCMV mutant P42 (FIG. 25B), LCMV mutant P52 (FIG. 25C), LCMV mutant P91 (FIG. 25D), LCMV mutant P52-1 (FIG. 25E), LCMV mutant P52-1.3 (FIG. 25F), and LCMV mutant P52-2.1 (FIG. 25G).

FIG. 26 A-H show alignments of nucleic acid and amino acid sequences LCMV-WE and mutants P42, P52, P92, P52-1, P52-1.3, and P52-2.1 pre-glycoprotein polyprotein GP complex (FIG. 26A and FIG. 26B), nucleoprotein (FIG. 26C and FIG. 26D), and ring finger protein Z (FIG. 26E and FIG. 26F), RNA-directed RNA polymerase L (FIG. 26G and FIG. 26H).

### OBJECT AND SOLUTION

The underlying object of the present invention is to produce arenaviruses with antitumor properties and/or improved antitumor properties. Furthermore, the underlying object of the invention is to provide corresponding arenavirus mutants, isolated proteins or peptides, in particular glycoproteins and/or L-proteins, of arenaviruses as well as nucleic acids coding for them.

The above objects are solved by a lymphocytic choriomeningitis virus mutant according to the invention, a method according to the invention, a lymphocytic choriomeningitis virus mutant a medicament or pharmaceutical composition according to the invention, an isolated protein or peptide according to the invention as well as an isolated nucleic acid according to the invention. Preferred embodiments of the method and the lymphocytic choriomeningitis virus mutant are subject of the dependent claims and to the present description. Additional aspects of the present invention are disclosed in the description. The wording of all claims is hereby incorporated by explicit reference to the content of the present description.

The present invention is based on the surprising finding that certain mutants of an arenavirus, in particular lymphocytic choriomeningitis virus (LCMV) may have improved antitumoral altivites as compared to a wild type virus.

Accordingly, the present invention generally relates to a mutant of lymphocytic choriomeningitis virus, preferably a mutant of strain WE.

The mutant may be capable of undergoing a stronger propagation in a tumor cell, such as a H1975 cell, a HCC1954 cell, a murine pancreatic cancer cell, or a human melanoma cell, as compared to the wild type lymphocytic choriomeningitis virus strain WE. The mutant may also be capable of inducing a stronger innate immune activation than LCMV-WE wild type in vivo. The mutant may also be capable of having a stronger antitumoral effect in vivo than LCMV-WE wild type. The mutant may also be capable of increasing expansion of tumor-specific CD8+ T cells as compared to LCMV-WE wild type. The mutant may also be capable of increasing the function of tumor-specific CD8+ T cells. The mutant may also have a higher capacity to stimulate tumor specific T cells as compared to the LCMV-WE wild type.

The mutant a of lymphocytic choriomeningitis virus of the invention preferably comprises a nucleic acid encoding a glycoprotein, wherein said glycoprotein comprises at least one mutation at positions corresponding to positions 181 and 185 of the wild type glycoprotein sequence set forth in SEQ ID NO: 10. The preferred mutations are Arg 185→Trp and/or Ile 181→Met (such as Arg 185→Trp only, Ile 181→Met only, or Arg 185→Trp and Ile 181→Met) as compared to the wild type glycoprotein sequence set forth in SEQ ID NO: 10 and/or (a) respective protein(s) encoded by the nucleic acid. While the examples of the present application demonstrate that the presence of either one of the mutations may already be sufficient in order to provide an improved function over a wild type lymphocytic choriomeningitis virus, the presence of both of the recited mutations will further improve the function of the LCMV, which may be additively or even synergistically. Accordingly, in a preferred embodiment, both mutations are present in the LCMV of the invention.

Without wishing to be bound by theory, it is further believed that the presence of the one or two above-mentioned mutations in the glycoprotein can improve the function of the LCMV (e.g. replication or propagation in a tumor cell, anti-tumoral activities, and/or immune system stimulation as described above), independent from the presence of other mutations, in particular of other mutations in other genes or proteins of LCMV. The assumption is based on the finding that the mutations Arg 185→Trp and Ile 181→Met in the glycoprotein remain conserved while some other mutations appear and disappear during serial passages, and/or are not conserved among different mutant strains. The assumption is further based on the fact that the glycoprotein(s) of LCMV form the spikes on the virion envelope, and are thus believed to play a predominant role in the virus's ability to infect a tumor cell.

The LCMV mutant of the invention may comprise a nucleic acid encoding a glycoprotein, wherein the glycoprotein has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.5%, preferably at least about 99.7% sequence identity, to the wild type glycoprotein sequence set forth in SEQ ID NO: 10, and/or may comprise (a) respective protein(s) encoded by the nucleic acid.

The LCMV mutant of the invention may comprises a nucleic acid encoding a glycoprotein, wherein the glycoprotein comprises the mutations Arg 185→Trp and Ile 181→Met as compared to the wild type glycoprotein set forth in SEQ ID NO: 10.

The LCMV mutant of the invention may comprise a nucleic acid encoding a glycoprotein, wherein said glycoprotein has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.5%, preferably at least about 99.7% sequence identity, or is preferably identical, to a sequence set forth in any one of SEQ ID NOs: 18, 26, 34, 42, 50, and 58, and/or may comprise (a) respective protein(s) encoded by the nucleic acid.

The LCMV mutant of the invention may comprise a nucleic acid encoding a glycoprotein, wherein said nucleic acid comprises a sequence that has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity, or is preferably identical, to a sequence set forth in SEQ ID NOs: 17, 25, 33, 41, 49, and 57, and/or may comprise (a) respective protein(s) encoded by the nucleic acid.

As already indicated above, it is believed that the glycoprotein(s) of LCMV, which form the spikes on the virion envelope, play a predominant role in the virus's ability to infect tumor cells. However, further proteins are preferably present in an LCMV.

The LCMV mutant of the invention may comprise a nucleic acid encoding a L-protein that has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity to the wild type L-protein sequence set forth in SEQ ID NO: 16, and/or may comprise a respective protein encoded by the nucleic acid.

The LCMV mutant of the invention may comprise a nucleic acid encoding a L-protein, wherein said L-protein has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, preferably at least about 99.8%, preferably at least about 99.9% sequence identity, or is preferably identical, to a sequence set forth in any one of SEQ ID NOs: 24, 32, 40, 48, 56, and 64, and/or may comprise a respective protein encoded by the nucleic acid.

The LCMV mutant of the invention may comprise a nucleic acid encoding a L-protein, wherein said L-protein comprises 1, 2, 3, 4, 5, 6, 7, 8, 9, or 10 mutations at positions corresponding to positions 253, 1512, 1513, 1758, 1995, 2094, 2115, 2141, 2175, and 2185 of the wild type L-protein sequence set forth in SEQ ID NO: 16. Preferred embodiments of mutations at these positions are: Lys 253→Arg; Lys 1512→Met; Lys 1513→Glu; Ser 1758→Phe; Phe 1995→Ser, Ile 2094→Val; Lys 2115→Glu; Thr 2141→Ala; Arg 2175→Lys; Thr 2185→Ala as compared to the wild type L-protein sequence set forth in SEQ ID NO: 16, and/or may comprise a respective protein encoded by the nucleic acid. Preferably, the LCMV mutein comprises 1, 2, 3, 4, or 5 of the aforementioned mutations.

The LCMV mutant of the invention may comprise a nucleic acid encoding a L-protein, wherein said L-protein comprises as compared to the wild type L-protein sequence set forth in SEQ ID NO: 16 one of the following sets of mutations: Ser 1758→Phe (corresponding to the mutations of mutant strain P42); Phe 1995→Ser, optionally Ile 2094→Val, and optionally Thr 2141→Ala (corresponding to the mutations of mutant strain P52, which is oligoclonal for positions 2094 and 2141); Lys 1513→Glu, Phe 1995→Ser, and optionally Arg 2175→Lys (corresponding to the mutations of mutant strain P91, which is oligoclonal for position 2175); Lys 253→Arg; Lys 1512→Met; Lys 2115→Glu; Thr 2185→Ala (corresponding to the mutations of mutant strain P52-1); Phe 1995→Ser (corresponding to the mutations of mutant strain P52-1.3); or Lys 2115→Glu (corresponding to the mutations of mutant strain P52-2.1), and/or may comprise a respective protein encoded by the nucleic acid.

The LCMV mutant of the invention may comprise a nucleic acid encoding an L-protein, wherein said nucleic acid has or—is preferably complementary to a sequence that has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, preferably at least about 99.8%, preferably at least about 99.9% sequence identity, or is preferably identical, to a sequence set forth in SEQ ID NOs: 17, 25, 33, 41, 49, and 57, and/or may comprise a respective protein encoded by the nucleic acid.

The LCMV mutant of the invention may comprise a nucleic acid encoding an nucleoprotein, wherein said nucleoprotein has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or is identical, to a nucleoprotein set forth in any one of SEQ ID NOs: 12, 20, 28, 36, 44, 52, and 60, and/or may comprise a respective protein encoded by the nucleic acid.

The LCMV mutant of the invention may comprise a nucleic acid encoding an nucleoprotein, wherein said nucleic acid has or—is preferably complementary to a sequence that has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or is identical, to a nucleoprotein set forth in any one of SEQ ID NOs: 11, 19, 27, 35, 43, 51, and 59, and/or may comprise a respective protein encoded by the nucleic acid.

The LCMV mutant of the invention may comprise a nucleic acid encoding a Z-protein, wherein said Z-protein has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or is identical, to a sequence set forth in any one of SEQ ID NOs: 14, 22, 30, 38, 46, 54, and 62, and/or may comprise a respective protein encoded by the nucleic acid.

The LCMV mutant of the invention may comprise a nucleic acid encoding a Z-protein, wherein said nucleic acid comprises a sequence that has at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity, or is identical, to a sequence set forth in any one of SEQ ID NOs: 13, 21, 29, 37, 45, 53, and 61, and/or may comprise a respective protein encoded by the nucleic acid.

A LCMV mutant of the invention may comprise (a) glycoprotein(s) preferably as defined herein, an L protein preferably as defined herein, a Z protein preferably as defined herein, and a nucleoprotein preferably as defined herein. A LCMV mutant of the invention may comprise (a) nucleic acid(s) that encode a) glycoprotein(s) preferably as defined herein, an L protein preferably as defined herein, an Z protein preferably as defined herein, and a nucleoprotein preferably as defined herein.

A LCMV mutant of the invention may comprise (a) nucleic acid(s) that encode the proteins of SEQ ID NOs: 18, 20, 22, and 24, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. The glycoprotein may comprise a Ile 181→Met mutation. The L-protein may comprise a Ser 1758→Phe mutation. A LCMV mutant of the invention may comprise (a) nucleic acid(s) that comprise(s) a sequence set forth in SEQ ID NOs: 17 and 21 and that comprise(s) a sequence that is complementary to the sequence set forth in SEQ ID NOs: 19 and 23.

A LCMV mutant of the invention may comprise (a) nucleic acid(s) that encode the proteins of SEQ ID NOs: 26, 28, 30, and 32, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. The glycoprotein may comprise a Ile 181→Met and a Arg 185→Trp mutation. The L protein may comprise following mutations Phe 1995→Ser, optionally Ile 2094→Val, and optionally Thr 2141→Ala. A LCMV mutant of the invention may comprise (a) nucleic acid(s) that comprise(s) a sequence set forth in SEQ ID NOs: 25 and 29 and that comprise(s) a sequence that is complementary to the sequence set forth in SEQ ID NOs: 27 and 31.

A LCMV mutant of the invention may comprise (a) nucleic acid(s) that encode the proteins of SEQ ID NOs: 34, 36, 38, and 40, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. The glycoprotein may comprise a Ile 181→Met and a Arg 185→Trp mutation. The L protein may comprise following mutations Lys 1513→Glu, Phe 1995→Ser, and optionally Arg 2175→Lys. A LCMV mutant of the invention may comprise (a) nucleic acid(s) that comprise(s) a sequence set forth in SEQ ID NOs: 33 and 37 and that comprise(s) a sequence that is complementary to the sequence set forth in SEQ ID NOs: 35 and 39.

A LCMV mutant of the invention may comprise (a) nucleic acid(s) that encode the proteins of SEQ ID NOs: 50, 52, 54, and 56, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. The glycoprotein may comprise a Ile 181→Met and a Arg 185→Trp mutation. The L protein may comprise following mutations Lys 253→Arg; Lys 1512→Met; Lys 2115→Glu; Thr 2185→Ala. A LCMV mutant of the invention may comprise (a) nucleic acid(s) that comprise(s) a sequence set forth in SEQ ID NOs: 41 and 45 and that comprise(s) a sequence that is complementary to the sequence set forth in SEQ ID NOs: 43 and 47.

A LCMV mutant of the invention may comprise (a) nucleic acid(s) that encode the proteins of SEQ ID NOs: 58, 60, 62, and 64, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. The glycoprotein may comprise a Arg 185→Trp mutation. The L protein may comprise a Phe 1995→Ser mutation. A LCMV mutant of the invention may comprise (a) nucleic acid(s) that comprise(s) a sequence set forth in SEQ ID NOs: 49 and 53 and that comprise(s) a sequence that is complementary to the sequence set forth in SEQ ID NOs: 51 and 55.

A LCMV mutant of the invention may comprise (a) nucleic acid(s) that encode the proteins of SEQ ID NOs: 66, 68, 70, and 72, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. The glycoprotein may comprise a Ile 181→Met mutation. The L protein may comprise a Lys 2115→Glu mutation. A LCMV mutant of the invention may comprise (a) nucleic acid(s) that comprise(s) a sequence set forth in SEQ ID NOs: 57 and 61 and that comprise(s) a sequence that is complementary to the sequence set forth in SEQ ID NOs: 59 and 63.

A LCMV of the disclosure may comprise (a) nucleic acid(s) that encode the proteins of SEQ ID NOs: 10, 12, 14, and 18, or proteins having at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7%, sequence identity to these sequences. A LCMV of the disclosure may comprise (a) nucleic acid(s) that comprise(s) a sequence set forth in SEQ ID NOs: 10 and 14 and that comprise(s) a sequence that is complementary to the sequence set forth in SEQ ID NOs: 12 and 16.

The present invention also generally relates to a LCMV of the disclosure, in particular of an LCMV mutant of the disclosure, for use in therapy.

In particular when a LCMV mutant of the disclosure is used, such use may comprise a stronger propagation of the LCMV mutant in a tumor (cell), as compared to the use wild type lymphocytic choriomeningitis virus strain WE. Such use may also comprise inducing a stronger innate immune activation as compared to the use of LCMV-WE wild type in vivo. Such use may also comprise promoting a stronger antitumoral effect in vivo as compared to the use of LCMV-WE wild type. Such use may also comprise increasing expansion of tumor-specific CD8+ T cells as compared to the use of LCMV-WE wild type. The use may also comprise increasing the function of tumor-specific CD8+ T cells. The use may also comprise stimulating tumor specific T cells to a larger extent as compared to the LCMV-WE wild type.

The LCMV (mutant) of the disclosure may be for use in the treatment and/or prevention of a tumor. The tumor may be any tumor disclosed herein. The tumor is preferably selected from the group consisting of carcinoma, melanoma, blastoma, lymphoma and sarcoma.

According to a first aspect, the invention relates to a method for the production of an antitumor arenavirus, i.e. a tumor-fighting or -repulsive arenavirus (so-called tumor regressive arenavirus), in particular an arenavirus which has in comparison to an original arenavirus improved antitumor properties, i.e. improved tumor-fighting or -repulsive properties.

The method comprises the following steps:
a) plating primary tumor cells in a nutrient medium, preferably liquid nutrient medium, or plating cells of the cell line H1975, C643 or Tramp-C2 onto and/or into a nutrient medium, preferably liquid nutrient medium,
b) inoculation of the plated primary tumor cells with an original arenavirus or inoculation of the plated cells of the cell line H1975, C643 or Tramp-C2 with an original arenavirus,
c) incubating the inoculated primary tumor cells or incubating the inoculated cells of the cell line H1975, C643 or Tramp-C2, i.e. incubating the primary tumor cells and the original arenavirus or incubating the cells of the cell line H1975, C643 or Tramp-C2 and the original arenavirus under conditions suitable for causing at least a portion of the inoculated primary tumor cells or at least a portion of the inoculated cells of the cell line H1975, C643 or Tramp-C2, in particular only part of the inoculated primary tumor cells or only part of the inoculated cells of the cell line H1975, C643 or Tramp-C2 or all inoculated primary tumor cells or all inoculated cells of the cell line H1975, C643 or Tramp-C2, to be infected with the original arenavirus,
d) extracting an arenavirus-containing cell culture supernatant from an incubated primary tumor cell culture, or extracting an arenavirus-containing cell culture supernatant from the incubated cells of the cell line H1975, C643 or Tramp-C2 containing cell culture,
wherein the step sequence a) to d) is repeated a plurality of times, wherein the primary tumor cells or the cells of the cell line H1975, C643 or Tramp-C2 when performing the first repetition of step sequence a) to d), for performing step b) is inoculated with the arenavirus-containing cell culture supernatant or a part thereof extracted when performing step d) before the first repetition of the step sequence a) to d, and wherein the primary tumor cells or the cells of cell line H1975, C643 or Tramp-C2 when performing each further repetition of the step sequence a) to d), for performing step b) is inoculated with the arenavirus-containing cell culture supernatant or a part thereof extracted when performing step d) of a previous repetition of the step sequence a) to d).

The first conduct of step sequence a) to d) can also be referred to as the “first passage” within the meaning of the present invention. The first passage is therefore performed with the original arenavirus as inoculum. Each further passage is then performed with an arenavirus-containing cell culture supernatant or part thereof extracted in step d) of a preceding preferably immediately preceding passage. From performing a second passage onwards said passage can be referred to as “repeated passage”, within the meaning of the present invention.

The term “original arenavirus” in the context of the present invention can be understood to mean an arenavirus which is used before the first repetition of step sequence a) to d), i.e. before the first passage for inoculation of the primary tumor cells or the cells of the cell line H1975, C643 or Tramp-C2 (step b)). The “original arenavirus” may refer to, which is described in more detail below, a wildtype arenavirus or a mutant thereof. In particular, the term may refer to an original arenavirus without antitumor properties or with antitumor properties.

The term “wild-type arenavirus” within the in the context of the present invention should be understood to mean an arenavirus with a genome which occurs in nature in its genetically normal form.

The term “primary tumor cells” in the context of the present invention should be understood to mean unpassaged or non-passaged tumor cells, i.e. tumor cells isolated directly from a tumor tissue or tumor cells isolated directly from a tumor tissue, which have been passaged before performing step a) a maximum of 1000 times, in particular a maximum of 100 times, preferably a maximum of 10 times, or primary tumor cells which are characterized by having different cell clones. In the case of the latter, heterogeneity can be demonstrated by different protein expression and by different DNA sequences (genetic fingerprinting).

The term “cell line H1975” in the context of the present invention should be understood to mean a cell line which has been isolated from a human adenocarcinoma and is deposited at ATCC (American Type Culture Collection) under the designation NCI-H1975 (ATCC® CRL-5908®) and under the accession or deposit number CVCL-1511.

The term “cell line C643” in the context of the present invention should be understood to mean a cell line which has been isolated from human anaplastic thyroid carcinoma deposited under the designation C643 and under the accession or deposit number CVCL-5969 (ExPASy Cellosaurus).

The term “cell line Tramp-C2” in the context of the present invention should be understood to mean a cell line isolated from a murine adenocarcinoma and deposited under the designation Tramp-C2 and under the accession or deposit number CVCL-3615 (ExPASy Cellosaurus).

The term “plating out” in the context of the present invention should be understood to mean the seeding or introduction of primary tumor cells or cells of the cell line H1975, C643 or Tramp-C2 onto and/or into a culture medium, preferably a liquid culture medium. In other words, the term “plating” in the sense of this invention should be understood to mean the cultivation, in particular initial cultivation, of primary tumor cells or cells of the cell line H1975, C643 or Tramp-C2 on and/or in a culture medium, preferably liquid culture medium.

The term “lymphocytic choriomeningitis virus WE strain” may refer to the LCMV-WE strain that has been described by Seiler, P., et al., J. Immunol. 162 (8), 4536-4541 (1999). The term “lymphocytic choriomeningitis virus WE strain” as used herein preferably refers to a LCMV that comprises glycoproteins having the sequence of positions 59-265 and 266-498 of SEQ ID NO: 10, a nucleoprotein having the sequence of SEQ ID NO: 12; a Z-protein having the sequence of SEQ ID NO: 14; and/or a L-protein having the sequence of SEQ ID NO: 16. The term “lymphocytic choriomeningitis virus WE strain” as used herein also preferably refers to a LCMV that comprises one ore more, preferably two nucleic acid molecules, which comprise sequences that are or are complementary to the sequences of SEQ ID NOs: 9, 11, 13, and/or 15.

The term “arenavirus mutant” or “mutant of an arenavirus” in the context of the present invention should be understood to mean an arenavirus whose genome and/or proteome has at least one mutation, in particular at least one point mutation, with respect to the genome and/or proteome of its wild type. Preferably, The term “arenavirus mutant” or “mutant of an arenavirus” should be understood as an arenavirus with a protein, in particular glycoprotein and/or L-protein, which has with respect to a corresponding protein, in particular glycoprotein and/or L-protein of a corresponding wildtype arenavirus, at least one mutation, preferably in the form of an amino acid substitution.

Accordingly, the term “lymphocytic choriomeningitis virus mutant” should be understood as a mutant of the lymphocytic choriomeningitis virus, whose genome and/or proteome has with respect to the genome and/or proteome of the wild-type lymphocytic choriomeningitis virus at least one mutation, in particular at least one point mutation. Preferably, the term “lymphocytic choriomeningitis virus mutant” should be understood to mean an arenavirus with a protein, in particular glycoprotein and/or L-protein, which has with respect to a corresponding protein, in particular glycoprotein and/or L-protein, of the wild-type lymphocytic choriomeningitis virus at least one mutation, preferably in the form of an amino acid substitution.

The term “glycoprotein” in the context of the present invention should be understood to mean a macromolecule which consists of a protein, which in a certain context can also be referred to as a protein moiety, or protein component and one or more covalently bound carbohydrate groups (sugar groups), in particular mono- and/or oligo- and/or polysaccharide groups.

The term “repeated passage” or “repeated passaging” in the context of the present invention, unless otherwise stated, should be understood as a single or repeated process in which primary tumor cells or cells of the cell line H1975, C643 or Tramp-C2 are treated with arenavirus produced from primary tumor cells or cells of the cell line H1975, C643 or Tramp-C2. Therefor a culture medium for primary tumor cells or cells of the cell line H1975 C643 or Tramp-C2, such as DMEM (Dulbecco's Modified Eagle's Medium) with 10% Fetal Calf Serum (FKS) and 1% penicillin/streptomycin/glutamine (equivalent to 1 unit/ml penicillin, 100 microg/ml streptomycin and 2 mM L-glutamine), must be used. Non-infected primary tumor cells or non-infected cells of the cell line H1975, C643 or Tramp-C2 are preferably plated 0 to 7 days, in particular 1 to 7 days, prior to the inoculation step (step b). Preferably, a culture medium used for cell plating is replaced prior to the inoculation step by a fresh culture medium. In some cases, the culture medium may be extracted 1, 10 or 100 minutes after the inoculation step (step b) and replaced with a fresh culture medium. After an incubation period of 24, 48, 72 or 96 hours, a cell culture supernatant containing an accumulation of mutated arenaviruses is extracted. Part of this supernatant is added to new, uninfected primary tumor cells or cells of the cell lines H1975, C643 or Tramp-C2.

The term “nucleic acid” as used herein may generally refers to DNA or RNA. DNA and RNA differ—among others—in their nucleobases. The complementary base to adenine in DNA is thymine, whereas in RNA, it is uracil. For the sake of simplification, the corresponding base to adenine is denoted as “t” throughout the application, which—depending on its context—may refer to thymine (in DNA) or uracil (in RNA).

The term “encoding” or “encode(s)” as used herein in the context of a nucleic acid, relates to a nucleic acid having a sequence that can be translated into a particular amino acid sequence that is encoded by the nucleic acid. The nucleic acid sequence may encompass the sequence of a coding strand, and may also encompass the sequence of a strand that is complementary to the coding strand.

“Percent (%) sequence identity” with respect to sequences disclosed herein is defined as the percentage of amino acid residues or nucleotides in a candidate sequence that are pair-wise identical with the amino acid residues or nucleotides in a reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is true for nucleotide sequences disclosed herein. For determining sequence identity, uracil (e.g. in RNA) may be considered to be identical to thymine (e.g. in DNA).

The invention is also based on the surprising finding that through passaging of primary tumor cells or cells of the cell line H1975, C643 or Tramp-C2 infected with an arenavirus, an arenavirus with antitumor properties, in particular an arenavirus with improved antitumor properties compared to the arenavirus that was used can be produced. The production of such an arenavirus is based on the fact that the original arenavirus, usually a wild-type arenavirus, is difficultly able to proliferate in the primary tumor cells or the cells of the cell line H1975. An equally limited proliferation was observed in the cell lines C643 and Tramp-C2. The limited proliferation triggers an increased selection or adaptation pressure in the arenavirus during infection and replication in the primary tumor cells or in the cells of the cell line H1975, C643 or Tramp-C2. Randomly emerged virus mutants, which were able to proliferate in the primary tumor cells or the cells of the cell line H1975, C643 or Tramp-C2, overgrow the original arenavirus, usually the wild type arenavirus. Through passaging, in particular a repeated passaging (multiple repetition of the step sequence a) to d)), preferably results in an enrichment of an arenavirus, which has an antitumoral effect with respect to the primary tumor cells or cells of the cell line H1975, C643 or Tramp-C2 or which has improved antitumoral properties as compared to the original arenavirus. The antitumoral effect of the arenavirus mutant is based on the fact that it can infect the cells better and replicates better within the cells. In doing so, the arenavirus mutant can spread more successfully in the cells used as compared to the original arenavirus and, in particular, promote enhanced immune activation. Since many tumor cells do not have any antiviral factors and therefore show a limited viral resistance, the method according to the invention can be particularly advantageous not only for the production of an antitumoral arenavirus, but also for the production of a tumor-specific arenavirus, i.e. for the production of an antitumoral and tumor-specific arenavirus.

The primary tumor cells as well as the cells of the cell lines H1975, C643 and Tramp-C2 were identified by the inventors as cells which surprisingly allow only a reduced replication of arenaviruses and which in the course of passing trigger a particularly high selection pressure or adaptation compulsion in arenaviruses.

In an embodiment of the invention, the primary tumor cells are passaged before performing step a) at most 1000 times, in particular at most 100 times, preferably at most 10 times. The term “passaging” shall in this context be understood as the dilution of the cells in a cell culture, thus the uptake of the cells into a suspension and plating of a part of the cells (e.g. 50%, 10% or 1%) in a new nutrient medium. The less the primary tumor cells are passaged before step a), the more the cells correspond to the properties of the tumor cells of a patient. In particular, large differences (heterogeneity) between individual primary tumor cells in morphology, RNA expression pattern, protein expression and DNA sequence map tumor cells in vivo. Thereby an arenavirus can be produced in an advantageous way, which is particularly suitable for a therapeutic treatment of respective tumor patients, in particular patient groups of it.

In particular, the primary tumor cells can prior to step a) be subjected to no passage. In other words, to perform step a) unpassaged, i.e. not passaged primary tumor cells can be used. Thereby the original arenavirus or the arenavirus contained in the extracted cell culture supernatant or a part thereof is forced to adapt to cells that represent the tumor cells of a patient particularly well. In doing so, it is possible to generate particularly suited arenaviruses for the therapeutic treatment of respective tumor patients, in particular patient groups thereof.

The already often passaged cells of the cell lines H1975, C643 and Tramp-C2, were identified by the inventors as equally suitable cells, although they have already been passaged in vitro for years.

In a further embodiment of the invention, step c) is performed during a period of 1 hour to 1000 hours, in particular 3 hours to 300 hours, preferably 12 hours to 96 hours. The time periods disclosed in this paragraph have been shown to be particularly beneficial for efficient incubation and consequently infecting of the inoculated primary tumor cells or the inoculated cells of the cell line H1975, C643 or Tramp-C2 with the original arenavirus or the arenavirus contained in the extracted cell culture supernatant or part thereof.

In a further embodiment of the invention, step (b) and/or step (c) is performed at a temperature of 4° C. to 50° C., in particular 20° C. to 42° C., preferably 34° C. to 39° C. The temperature ranges disclosed in this paragraph have been found to be particularly advantageous for efficient inoculation and/or incubation (and consequently infection) of the primary tumor cells or cells of the cell line H1975, C643 or Tramp-C2 with the original arenavirus or the arenavirus contained in the extracted cell culture supernatant or part thereof.

In a further embodiment of the invention, step a) and/or step b) and/or step c) and/or step d) is performed in a nutrient medium preferably selected from the group consisting of RPMI-1640 (Roswell Park Memorial Institute), DMEM (Dulbecco's Modified Eagle's Medium) and IMDM (Iscove's Modified Dulbecco's Medium). The nutrient medium may additionally contain serum (0.1-20%), such as fetal calf serum and/or human serum, and/or amino acids, such as glutamate and/or glutamine, and/or antibiotics. A culture medium enables the cultivation of the primary tumor cells or the cells of cell line H1975, C643 or Tramp-C2. In particular, a culture medium favours with particular advantage the growth and/or cell division of the primary tumor cells or cells of the cell line H1975, C643 or Tramp-C2. The culture media mentioned in this paragraph are particularly suitable for the cultivation of primary tumor cells or cells of the cell line H1975, C643 or Tramp-C2.

In a further embodiment of the invention, step a) and/or step b) and/or step c) are performed under a carbon dioxide atmosphere (CO2 atmosphere) of 0% to 20%, in particular 0.1% to 20%, preferably 2% to 10%, more preferably 4% to 6%. This allows the pH of a nutrient medium used for step a) and/or step b) and/or step c) to be kept constant. This allows efficient plating and/or inoculation and/or incubation (and consequently infection) of the primary tumor cells or the cells of the cell line H1975, C643 or Tramp-C2 with the original arenavirus respectively the arenavirus contained in the extracted cell culture supernatant or part thereof.

Preferably step a) and/or step b) and/or step c) is performed in an incubator, by means of which controlled external conditions for growth and/or cell division of the primary tumor cells or the cells of cell line H1975, C643 or Tramp-C2 can be created.

In the further embodiment of the invention, the sequence of steps a) to d) is repeated 3 times to 1000 times, in particular 10 times to 100 times, preferably 20 times to 50 times. A multiple repetition of the step sequence a) to d), in particular as disclosed in this paragraph, has been shown to be particularly beneficial in view of the development of beneficial mutations with regard to antitumoral properties, in particular point mutations, and consequently to the production of an antitumoral arenavirus.

In the further embodiment of the invention, the nutrient medium is replaced by a fresh or new nutrient medium before performing step b). Through this step the conditions of infection can advantageously be standardized.

In a further embodiment of the invention, the culture medium is replaced by a fresh or new culture medium within a period of 0.1 minutes to 600 minutes, in particular 1 minute to 30 minutes, preferably 5 minutes to 15 minutes after step b). This step advantageously increases the selection pressure of the infection, as the time of infection is limited.

In a further embodiment of the invention multiple nutrient medium changes are performed. For example, the nutrient medium can be replaced by a fresh or new nutrient medium before step b) and the latter can be replaced by a fresh or new nutrient medium within a period of 0.1 minutes to 600 minutes, in particular 1 minute to 30 minutes, preferably 5 minutes to 15 minutes after performing step b).

In a further embodiment of the invention, the primary tumor cells or the cells of the cell line H1975, C643 or Tramp-C2 and/or the original arenavirus and/or the arenavirus contained in the extracted cell culture supernatant or part thereof are treated with at least one chemotherapeutic agent before performing step d), in particular before performing step c), in particular before performing step b), in particular before performing step a). Thereby tumor cells can be simulated which have already been chemotherapeutically treated. This advantageously allows the production of an arenavirus which is particularly suited for the treatment of tumor patients who have already been treated with chemotherapy and/or who are considered to have undergone all therapy options already.

In the further embodiment of the invention, the original arenavirus and/or the arenavirus contained in the extracted cell culture supernatant or a part thereof is before performing step b) treated with at least one chemotherapeutic agent. Thereby the natural mutation rate in the arenavirus can advantageously be increased and thus accelerating its adaptation to the primary tumor cells or cells of the cell line H1975, C643 or Tramp-C2.

The at least one chemotherapeutic agent may in particular be selected from the group consisting of alkylants, topoisomerase inhibitors, mitotic inhibitors, antimetabolites, antibiotics, kinase inhibitors, thalidomide derivatives, cell apoptosis inducers, biological therapeutics such as biological cytostatics, isotope-containing compounds, hormones, hormone antagonists, histone deacetylase inhibitors and other cytostatic agents.

The alkylating agents may be selected from the group consisting of oxazaphosphorins, N-lost derivatives, alkyl sulfonates, hydrazines, platinum-containing substances, anthracyclines and mixtures of at least two of the above alkylating agents.

The oxazaphosphorins may, for example, be selected from the group consisting of cyclophosphamide, ifosfamide and mixtures thereof.

The N-lost derivatives may be selected from the group consisting of chlorambucil, melphalan and mixtures thereof.

The alkyl sulfonates may, for example, be husulfan.

The hydrazines may, for example, be selected from the group consisting of temozolomide, dacarbazine, procarbazine and mixtures of at least two of the above hydrazines.

The platinum-containing substances may, for example, be selected from the group consisting of cisplatin, carboplatin, oxaliplatin and mixtures of at least two of said platinum-containing substances.

The anthracyclines may, for example, be selected from the group consisting of doxorubicin, daunorubicin, idarubicin, elirubicin and mixtures of at least two of said anthracyclines.

The above-mentioned topoisomerase inhibitors may be selected from the group consisting of topoisomerase I inhibitors, topoisomerase II inhibitors (etoposide) and mixtures thereof.

For example, the topoisomerase I inhibitors may be selected from the group consisting of irinotecan, topotecan and mixtures thereof.

Topoisomerase II inhibitors may be etoposide, for example.

The above-mentioned mitosis inhibitors or antimetabolites may be selected from the group consisting of vinca alkaloids, taxanes, folic acid antagonists, pyrimidine antagonists, purine antagonists, ribonucleotide reductase inhibitors and mixtures of at least two of the above-mentioned mitosis inhibitors or antimetabolites.

For example, the vinca alkaloids may be selected from the group consisting of vincristine, vinblastine and mixtures thereof.

Taxanes may be selected from the group consisting of docetaxel, paclitaxel and mixtures thereof.

For example, the folic acid antagonists may be selected from the group consisting of methotrexate, pemetrexed and mixtures thereof.

The pyrimidine antagonists may, for example, be selected from the group consisting of cytarabine, 5-fluorouracil, gemcitabine, capecitabine and mixtures of at least two of said pyrimidine antagonists.

The purine antagonists may, for example, be selected from the group consisting of 5-azacytidine, azathioprine, 6-mercaptopurine, fludarabine and mixtures of at least two of said purine antagonists.

For example, the ribonucleotide reductase inhibitor may be hydroxyurea.

The above antibiotics may be selected from the group consisting of bleomycin, actinomycin D, mitomycin and mixtures of at least two of the above antibiotics.

The above mentioned kinase inhibitors may be selected from the group consisting of afatinib, alectinib, axitinib, crizotinib, cobimetinib, dasatinib, dabrafenib, erlotinib, gefitinib, imatinib, Ixazomib, lenvatinib, nilotinib, osimertinib, palbociclib, pazopanib, ponatinib, regorafenib, sunitinib, vemurafenib, trametinib, everolimus and mixtures of at least two of said kinase inhibitors.

The above mentioned thalidomide derivatives may be selected from the group consisting of lenalidomide, pomalidomide and mixtures thereof.

The cell apoptosis inducers mentioned above may be selected from the group consisting of methoxals, venetoclax and mixtures thereof.

The above mentioned biological therapeutics may be selected from the group consisting of rituximab, trastuzumab, cetuximab, panitumumab, ipilimumab, pembrolizumab, nivolumab, atezolizumab, avelumab, nivolumab, olaratumab, ramucirumab and mixtures of at least two of the above mentioned biological therapeutics.

The above-mentioned hormones and/or hormone antagonists may be selected from the group consisting of buserelin, goserelin, leuprorelin, triptorelin, estramustin, tamoxifen, aromatase inhibitors such as anastrozole, Antiandrogens such as enzalutamide, flutamide, bica-lutamide, progestins such as megestrol acetate and medroxyprogesterone acetate, glucocorticoids and mixtures of at least two of the aforementioned hormones and/or hormone antagonists.

The other cytostatic agents mentioned above may be selected from the group consisting of bexarotene, afatinib, crizotinib erlotinib, gefitinib, lapatinib, dasatinib, imatinib, nilotinib, ponatinib, regorafenib, sonidegib, hydroxycarbamide, trametinib, tretininoin, isotretinoin, alitretinoin, MAOP (5-amino-4-oxopentanoic acid methyl ester) and mixtures of at least two of the foregoing other cytostatic agents.

Furthermore, the primary tumor cells or the cells of the cell line H1975, C643 or Tramp-C2 and/or the original arenavirus and/or the arenavirus contained in the extracted cell culture supernatant or a part thereof, before performing step d), in particular before performing step c), in particular before performing step b), in particular before performing step a), may be treated with radiation, in particular selected from the group consisting of ultraviolet (UV) rays, in particular UVA rays and/or UVB rays, alpha rays, beta rays, gamma rays and X-rays. Thereby, tumor cells can advantageously be simulated which have already been subjected to therapeutic radiation. This advantageously allows the production of an arenavirus, which can be used especially for the treatment of tumor patients who have already been treated with radiation therapy.

Furthermore, the original arenavirus and/or the arenavirus contained in the extracted cell culture supernatant or part thereof before performing step b) be treated with radiation, in particular selected from the group consisting of ultraviolet (UV) rays, in particular UVA rays and/or UVB rays, alpha rays, beta rays, gamma rays and X-rays. Thereby the natural mutation rate in the arenavirus and thus its adaptation to the primary tumor cells or cells of the cell line H1975, C643 or Tramp-C2 can advantageously be increased.

In another embodiment of the invention primary tumor cells or cells of the cell line H1975, C643 or Tramp-C2 are used which are resistant to at least one chemotherapeutic agent. For example, primary tumor cells resistant to paclitaxel and/or trametinib may be used. Thereby, tumor cells of a patient can advantageously be simulated, which are resistant to at least one chemotherapeutic agent during tumor treatment. The embodiments of the invention described in this paragraph thus represent further possibilities for the production of a powerful antitumoral arenavirus.

In a further embodiment of the invention, the primary tumor cells or the cells of the cell line H1975, C643 or Tramp-C2 and/or the original arenavirus and/or the arenavirus contained in the extracted cell culture supernatant or a part thereof are before performing step d), in particular before performing step c), in particular before performing step b), in particular before performing step a) treated with at least one antiviral compound, in particular with alpha-interferon and/or gamma-interferon. Thereby original arenavirus or the arenavirus contained in the cell culture supernatant extracted or in a part thereof can advantageously be forced to adapt in an antiviral environment, whereby an antitumoral arenavirus exhibiting resistance at the same time can be produced. Such an arenavirus is particularly effective from a therapeutic point of view, as it can also be used to combat tumor tissue that may exhibit viral resistance.

Furthermore, the method may comprise a further step e) isolating and/or identifying an antitumoral arenavirus, in particular an arenavirus, which has improved antitumoral properties with respect to the original arenavirus, from the cell culture supernatant extracted or a part thereof by means of cloning and/or PCR (polymerase chain reaction) and/or sequencing.

Primary tumor cells preferably used are primary malignant tumor cells, in particular primary carcinoma cells, primary melanoma cells, primary blastoma cells, primary lymphoma cells or primary sarcoma cells.

In a further embodiment of the invention, primary tumor cells are primary choroidal melanoma cells, primary anal carcinoma cells, primary angiosarcoma cells, primary astrocytoma cells, primary basal cell carcinoma cells, primary cervical carcinoma cells, primary chondrosarcoma cells, primary chorionic carcinoma cells, primary dermal squamous cell carcinoma cells, primary small intestine carcinoma cells, primary endometrial carcinoma cells, primary Ewing sarcoma cells, primary fibrosarcoma cells, primary gallbladder carcinoma cells, primary bile duct carcinoma cells, primary glioblastoma cells, primary bladder carcinoma cells, primary ureter carcinoma cells, primary urethral carcinoma cells, primary hepatocellular carcinoma cells, primary testicular tumor cells, primary hypopharyngeal carcinoma cells, primary pituitary carcinoma cells, primary Kaposi sarcoma cells, primary small cell bronchial carcinoma cells, primary colon carcinoma cells, primary colorectal carcinoma cells, primary laryngeal carcinoma cells, primary leiomyosarcoma cells, primary liposarcoma cells, primary gastric carcinoma cells, primary malignant fibrous histiocytoma cells, primary breast carcinoma cells, primary medulloblastoma cells, primary melanoma cells, primary oral floor carcinoma cells, primary sinus carcinoma cells, primary nasopharyngeal carcinoma cells, primary adrenal cortex carcinoma cells, primary parathyroid carcinoma cells, primary neurogenic sarcoma cells, primary non-small-cell bronchial carcinoma cells, primary renal carcinoma cells, primary oropharyngeal carcinoma cells, primary osteosarcoma cells, primary ovarian carcinoma cells, primary pancreatic tumor cells, primary penis carcinoma cells, primary pheochromocytoma cells, primary pleural mesothelioma cells, primary prostate carcinoma cells, primary rectal carcinoma cells, primary retinoblastoma cells, primary rhabdomyosarcoma cells, primary thyroid carcinoma cells, primary salivary gland carcinoma cells, primary esophageal carcinoma cells, primary tonsil carcinoma cells, primary vaginal carcinoma cells, primary vulvar carcinoma cells, primary Wilms tumor cells, primary cells of neuroendocrine tumors or primary tongue carcinoma cells.

Particularly preferred are primary melanoma cells, primary lung carcinoma cells, primary pancreatic carcinoma cells, primary colon carcinoma cells, primary gastric carcinoma cells, primary pharyngeal carcinoma cells, primary laryngeal carcinoma cells, primary renal cell carcinoma cells are particularly preferred as primary tumor cells, primary ovarian carcinoma cells, primary endometrial carcinoma cells, primary thyroid carcinoma cells, primary prostate carcinoma cells, primary liver carcinoma cells or primary sarcoma cells, such as primary neurogenic sarcoma cells, primary osteosarcoma cells or primary rhabdomyosarcoma cells.

In a further embodiment of the invention, a wild type arenavirus, i.e. a so-called wild type arenavirus, is used as the original arenavirus.

In a further embodiment of the invention, an old world arenavirus is used as the original arenavirus. The old world arenavirus is preferably selected from the group consisting of Catarina Virus, Danfenong Virus, Ippy Virus (IP-PYV), Kodoko Virus, Lassa Virus (LASV), Lymphocytic Choriomeningitis Virus (LCMV), Morogoro Virus, Mobala Virus (MOBV), Gairo Virus and Mopeia Virus (MOPV), Pinhal Virus, Skinner Tank Virus.

Preferred is as original arenavirus the lymphocytic choriomeningitis virus, in particular a wild type of the lymphocytic choriomeningitis virus. For example, a strain of the lymphocytic choriomeningitis virus, in particular selected from the group consisting of WE, Armstrong, Clone 13 (Clone 13) and Docile, can be used as the original arenavirus.

Particularly preferred is the wild-type lymphocytic choriomeningitis virus, i.e. a wild-type lymphocytic choriomeningitis virus comprising an L-protein comprising or consisting of an amino acid sequence according to SEQ ID NO: 7, and/or a nucleic acid, in particular ribonucleic acid, preferably L-ribonucleic acid, comprising or consisting of a nucleic acid sequence, in particular ribonucleic acid sequence, preferably L-ribonucleic acid sequence, which is complementary to a nucleic acid sequence pursuant to SEQ ID NO: 8.

Furthermore, a new world arenavirus can be used as original arenavirus. The new world arenavirus is preferably selected from the group consisting of Allpahuayo Virus (ALLV), Amapari Virus (AMAV), Bear Canyon Virus (BCNV), Chapare Virus, Cupixi Virus (CPXV), Flexal Virus (FLEV), Guanarito Virus (GTOV), Junin Virus (JUNV), Candid #1 (Candid No. 1).), Latino virus (LATV), Machupo virus (MACV), Oliveros virus (OLVV), Parana virus (PARV), Pichinide virus (PICV), Pirital virus (PIRV), Sabia virus (SABV), Tacaribe virus (TCRV), Tamiami virus (TAMV) and Whitewater arroyo virus (WWAV).

Furthermore, an arenavirus without antitumoral properties can be used as the original arenavirus.

Alternatively, an original arenavirus with antitumoral properties can be used.

According to a second aspect, the invention relates to a lymphocytic choriomeningitis virus mutant, i.e. a mutant of the lymphocytic choriomeningitis virus.

The lymphocytic choriomeningitis virus mutant comprises a protein or peptide, in particular a glycoprotein. The protein or peptide comprises a mutation, in particular a point mutation. The mutation, in particular point mutation, is preferably different from the sequences AJ297484, AJ233196 and the reference sequence LCMV, (strain WE-Essen).

Preferably the lymphocytic choriomeningitis virus mutant has a glycoprotein, in particular a protein component or a protein moiety of a glycoprotein, with at least one mutation, wherein the at least one mutation is an amino acid substitution of the isoleucine at position 181 of the glycoprotein by another amino acid, preferably methionine, and/or an amino acid substitution of the arginine at position 185 of the glycoprotein by another amino acid, preferably tryptophan.

Alternatively or in combination, the lymphocytic choriomeningitis virus mutant preferably comprises an L-protein with at least one mutation, wherein the at least one mutation is an amino acid substitution of the lysine at position 1513 of the L-protein by another amino acid, preferably glutamate, and/or an amino acid substitution of the phenylalanine at position 1995 of the L-protein by another amino acid, preferably serine, and/or an amino acid substitution of isoleucine at position 2094 of the L-protein by another amino acid, preferably valine, and/or an amino acid substitution of threonine at position 2141 of the L-protein by another amino acid, preferably alanine, and/or an amino acid substitution of arginine at position 2175 of the L-protein by another amino acid, preferably lysine.

Alternatively or in combination, the lymphocytic choriomeningitis virus mutant preferably comprises a protein or peptide, in particular a glycoprotein, in particular a protein component or a protein moiety of a glycoprotein, or an L-protein, comprising or consisting of an amino acid sequence pursuant to SEQ ID NO: 1, SEQ ID NO: 3, SEQ ID NO: 5, SEQ ID NO: 18, SEQ ID NO: 26, SEQ ID NO: 34, SEQ ID NO: 42, SEQ ID NO: 50, SEQ ID NO: 58, SEQ ID NO: 24, SEQ ID NO: 32, SEQ ID NO: 40, SEQ ID NO: 48, SEQ ID NO: 56, and SEQ ID NO: 64. The amino acid sequences according to SEQ ID NO: 1, SEQ ID NO: 3, SEQ ID NO: 18, SEQ ID NO: 26, SEQ ID NO: 34, SEQ ID NO: 42, SEQ ID NO: 50, and SEQ ID NO: 58 are preferably amino acid sequences of a glycoprotein of the lymphocytic choriomeningitis virus mutant. The amino acid sequence of SEQ ID NO: 5, SEQ ID NO: 24, SEQ ID NO: 32, SEQ ID NO: 40, SEQ ID NO: 48, SEQ ID NO: 56, and SEQ ID NO: 64 are preferably the amino acid sequence of an L-protein of the lymphocytic choriomeningitis virus mutant.

Alternatively or in combination, the lymphocytic choriomeningitis virus mutant preferably comprises a nucleic acid, in particular ribonucleic acid, which encodes a protein or peptide, in particular a glycoprotein or L-protein, comprising or consisting of an amino acid sequence according to SEQ ID NO: 1, SEQ ID NO: 3, SEQ ID NO: 5, SEQ ID NO: 18, SEQ ID NO: 26, SEQ ID NO: 34, SEQ ID NO: 42, SEQ ID NO: 50, SEQ ID NO: 58, SEQ ID NO: 24, SEQ ID NO: 32, SEQ ID NO: 40, SEQ ID NO: 48, SEQ ID NO: 56, or SEQ ID NO: 64.

Alternatively or in combination, the lymphocytic choriomeningitis virus mutant preferably comprises a nucleic acid, in particular ribonucleic acid, which comprises a nucleic acid sequence, in particular ribonucleic acid sequence, or consists of a nucleic acid sequence, in particular ribonucleic acid sequence, which is complementary to a nucleic acid sequence pursuant to SEQ ID No. 2, SEQ ID No. 4, SEQ ID No. 6, SEQ ID NO: 17, SEQ ID NO: 25, SEQ ID NO: 33, SEQ ID NO: 41, SEQ ID NO: 49, SEQ ID NO: 57, SEQ ID NO: 23, SEQ ID NO: 31, SEQ ID NO: 39, SEQ ID NO: 47, SEQ ID NO: 55, or SEQ ID NO: 63.

It was surprisingly found out that a respective mutant/respective mutants of the lymphocytic choriomeningitis virus—as described in the previous paragraphs—has/have improved replication competence in primary tumor cells or cells of the cell line H1975 as well as antitumoral or improved antitumoral properties, in particular with respect to the wild type of the lymphocytic choriomeningitis virus.

Furthermore, it is preferred that the codon for isoleucine at position 181 of the glycoprotein is replaced by another codon, preferably by a codon for methionine.

Furthermore, it is preferred that the codon for arginine at position 185 of the glycoprotein is replaced by another codon, preferably by a codon for tryptophan.

Furthermore, it is preferred that the codon for histidine at position 155 of the glycoprotein is replaced by another codon, preferably a codon for tyrosine.

Furthermore, it is preferred that the codon for arginine at position 358 of the glycoprotein is replaced by another codon, preferably by a codon for lysine.

Furthermore, it is also preferred that the lymphocytic choriomeningitis virus mutant comprises a nucleic acid, in particular ribonucleic acid, preferably L-ribonucleic acid, comprising a mutation, wherein the mutation is a nucleotide substitution of adenine at position 4537 of the nucleic acid, in particular ribonucleic acid, preferably L-ribonucleic acid, by guanine.

Furthermore, it is preferred that the lymphocytic choriomeningitis virus mutant comprises a nucleic acid, in particular ribonucleic acid, preferably L-ribonucleic acid, encoding an L-protein comprising a mutation, wherein the mutation is an amino acid substitution of the lysine at position 1513 of the L-protein, by another amino acid, preferably by glutamate.

Furthermore, it is preferred that the lymphocytic choriomeningitis virus mutant comprises a nucleic acid, in particular a ribonucleic acid, preferably L-ribonucleic acid, encoding an L-protein comprising a mutation, wherein the mutation is an amino acid substitution of the phenylalanine at position 1995 of the L-protein, by another amino acid, preferably by a serine.

Furthermore, it is also preferred that the lymphocytic choriomeningitis virus mutant comprises a nucleic acid, in particular ribonucleic acid, preferably L-ribonucleic acid, comprising a mutation, wherein the mutation is a nucleotide substitution of thymine at position 5984 of the nucleic acid, in particular ribonucleic acid, preferably L-ribonucleic acid, preferably by the nucleotide cytosine.

Furthermore, it is also preferred that the lymphocytic choriomeningitis virus mutant comprises a nucleic acid, in particular ribonucleic acid, preferably L-ribonucleic acid, which encodes an L-protein comprising a mutation, wherein the mutation is an amino acid substitution of the isoleucine at position 2094 of the L-protein, by another amino acid, preferably by valine.

Furthermore, it is preferred that the lymphocytic choriomeningitis virus mutant comprises a nucleic acid, in particular ribonucleic acid, preferably L-ribonucleic acid, comprising a mutation, wherein the mutation is a nucleotide substitution of adenine at nucleotide position 6280 of the nucleic acid, in particular ribonucleic acid, preferably L-ribonucleic acid, by another nucleotide, preferably guanine.

Furthermore, it is preferred that the lymphocytic choriomeningitis virus mutant comprises a nucleic acid, in particular ribonucleic acid, preferably L-ribonucleic acid, encoding an L-protein comprising a mutation, wherein the mutation is an amino acid substitution of the threonine at position 2141 of the L-protein, by another amino acid, preferably by alanine.

Furthermore, it is also preferred that the lymphocytic choriomeningitis virus mutant comprises a nucleic acid, in particular ribonucleic acid, preferably L-ribonucleic acid, comprising a mutation, wherein the mutation is a nucleotide substitution of adenine at position 6421 of the nucleic acid, in particular ribonucleic acid, preferably L-ribonucleic acid, preferably by another nucleotide, preferably guanine.

Furthermore, it is also preferred that the lymphocytic choriomeningitis virus mutant comprises a nucleic acid, in particular ribonucleic acid, preferably L-ribonucleic acid, encoding an L-protein comprising a mutation, wherein the mutation is an amino acid substitution of arginine at position 2175 of the L-protein, preferably by lysine.

Furthermore, it is also preferred that the lymphocytic choriomeningitis virus mutant comprises a nucleic acid, in particular ribonucleic acid, preferably L-ribonucleic acid, comprising a mutation, wherein the mutation is a nucleotide substitution of guanine at position 6524 of the nucleic acid, in particular ribonucleic acid, preferably L-ribonucleic acid, by the nucleotide adenine.

In another embodiment of the invention the lymphocytic choriomeningitis virus mutant is a lymphocytic choriomeningitis virus mutant, including any lymphocytic choriomeningitis virus mutant of the disclosure, for application or use in medicine.

Preferably the lymphocytic choriomeningitis virus mutant is a lymphocytic choriomeningitis virus mutant for application or use in the treatment and/or prevention of a tumor.

The tumor is preferably selected from the group consisting of carcinoma, melanoma, blastoma, lymphoma and sarcoma.

The term “carcinoma” in the context of the present invention should be understood to mean a malignant neoplasia of epithelial origin.

The term “sarcoma” in the context of the present invention should be understood to mean a malignant neoplasia of mesodermal origin.

The term “melanoma” in the context of the present invention should be understood to mean a malignant neoplasia of melanocytic origin.

The term “lymphoma” in the context of the present invention should be understood to mean a malignant neoplasia of lymphocytic origin.

The term “blastoma” in the context of the present invention should be understood to mean a malignant neoplasia of embryonic origin.

The carcinoma is preferably selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, oropharyngeal carcinoma, prostate carcinoma, thyroid carcinoma and cervical carcinoma.

The sarcoma can be selected from the group consisting of angiosarcoma, chondrosarcoma, Ewing sarcoma, fibrosarcoma, Kaposi sarcoma, liposarcoma, leiomyosarcoma, malignant fibrous histiocytoma, neurogenic sarcoma, osteosarcoma and rhabdomyosarcoma.

Further, the lymphocytic choriomeningitis virus mutant can be prepared or used for local, in particular intramuscular, intraperitoneal, or subcutaneous administration.

Alternatively, the lymphocytic choriomeningitis virus mutant can be prepared or used for systemic, in particular intravenous, administration.

For further characteristics and benefits of the lymphocytic choriomeningitis virus mutant, full reference is made to the previous and the following description.

According to a third aspect, the invention relates a lymphocytic choriomeningitis virus mutant comprising or consisting of a protein or peptide, in particular glycoprotein or L protein, comprising or consisting of an amino acid sequence pursuant to SEQ ID NO: 1, SEQ ID NO: 3, SEQ ID NO: 5, SEQ ID NO: 18, SEQ ID NO: 26, SEQ ID NO: 34, SEQ ID NO: 42, SEQ ID NO: 50, SEQ ID NO: 58, SEQ ID NO: 66, SEQ ID NO: 24, SEQ ID NO: 32, SEQ ID NO: 40, SEQ ID NO: 48, SEQ ID NO: 56, or SEQ ID NO: 64.

Alternatively or in combination, the lymphocytic choriomeningitis virus mutant comprises a nucleic acid, in particular ribonucleic acid, wherein the nucleic acid, in particular ribonucleic acid, comprises a nucleic acid sequence, in particular ribonucleic acid sequence, or consists of a nucleic acid sequence, in particular ribonucleic acid sequence, which is or is complementary to a nucleic acid sequence pursuant to SEQ ID NO: 2, SEQ ID NO: 4, SEQ ID NO: 6, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 23, SEQ ID NO: 31, SEQ ID NO: 39, SEQ ID NO: 47, SEQ ID NO: 55, or SEQ ID NO: 63.

Preferably the lymphocytic choriomeningitis virus mutant is a lymphocytic choriomeningitis virus mutant for application or use in medicine.

The lymphocytic choriomeningitis virus mutant is particular preferably a lymphocytic choriomeningitis virus mutant for use or application in the treatment and/or prevention of a tumor.

The tumor is preferably selected from the group consisting of carcinoma, melanoma, blastoma, lymphoma and sarcoma.

The term “carcinoma” in the context of the present invention should be understood to mean a malignant neoplasia of epithelial origin.

The term “sarcoma” in the context of the present invention should be understood to mean a malignant neoplasia of mesodermal origin.

The term “melanoma” in the context of the present invention should be understood to mean a malignant neoplasia of melanocytic origin.

The term “lymphoma” in the context of the present invention should be understood to mean a malignant neoplasia of lymphocytic origin.

The term “blastoma” in the context of the present invention should be understood to mean a malignant neoplasia of embryonic origin.

The carcinoma is preferably selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, oropharyngeal carcinoma, prostate carcinoma, thyroid carcinoma and cervical carcinoma.

The sarcoma can be selected from the group consisting of angiosarcoma, chondrosarcoma, Ewing's sarcoma, fibrosarcoma, Kaposi's sarcoma, liposarcoma, leiomyosarcoma, malignant fibrous histiocytoma, neurogenic sarcoma, osteosarcoma and rhabdomyosarcoma.

Further, the lymphocytic choriomeningitis virus mutant can be prepared or used for local, in particular intramuscular, intraperitoneal, or subcutaneous administration.

Alternatively, the lymphocytic choriomeningitis virus mutant can be prepared or used for systemic, in particular intravenous, administration.

With regard to other characteristics and advantages of the lymphocytic choriomeningitis virus mutant, in order to avoid repetitions, full reference is also made to the previous description, in particular to the statements made in the context of the second aspect of the invention. The features and advantages described therein, in particular with regard to the lymphocytic choriomeningitis virus mutant, the protein or peptide, in particular glycoprotein, and the nucleic acid, in particular ribonucleic acid, also apply mutatis mutandis to the lymphocytic choriomeningitis virus mutant according to the third aspect of the invention.

According to a fourth aspect, the invention relates to a drug or medicament which comprises a lymphocytic choriomeningitis virus mutant according to the second or third aspect of the invention.

The drug or medicament can further comprise a checkpoint blocker, such as PD-1 (Programmed Cell Death Protein 1), and/or an apoptosis modulator, in particular an apoptosis inhibitor, such as SMAC-mimeticum (LCL-161).

The drug or medicament preferably further comprises a pharmaceutically acceptable carrier. The carrier may be selected from the group consisting of water, aqueous saline solution, aqueous buffer solution, cell culture medium and combinations of at least two of the foregoing carriers.

With regard to further features and advantages of the drug or medicament, in order to avoid repetitions, full reference is made to the previous description, in particular to the statements made in the context of the second and third aspects of the invention. The features and advantages described therein, in particular with respect to the lymphocytic choriomeningitis virus mutant, also apply mutatis mutandis to the drug or medicament referred to in the fourth aspect of the invention.

According to a fifth aspect, the invention relates an protein or peptide, which is preferably an isolated protein or peptide, in particular a glycoprotein or an L-protein, comprising or consisting of an amino acid sequence according to SEQ ID NO: 1, SEQ ID NO: 3, SEQ ID NO: 5, SEQ ID NO: 7, SEQ ID NO: 10, SEQ ID NO: 18, SEQ ID NO: 26, SEQ ID NO: 34, SEQ ID NO: 42, SEQ ID NO: 50, SEQ ID NO: 58, SEQ ID NO: 24, SEQ ID NO: 32, SEQ ID NO: 40, SEQ ID NO: 48, SEQ ID NO: 56, or SEQ ID NO: 64. The invention also relates to an isolated protein or peptide having at least at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity to SEQ ID NO: 1, SEQ ID NO: 3, SEQ ID NO: 5, SEQ ID NO: 7, SEQ ID NO: 10, SEQ ID NO: 18, SEQ ID NO: 26, SEQ ID NO: 34, SEQ ID NO: 42, SEQ ID NO: 50, SEQ ID NO: 58, SEQ ID NO: 24, SEQ ID NO: 32, SEQ ID NO: 40, SEQ ID NO: 48, SEQ ID NO: 56, or SEQ ID NO: 64.

The amino acid sequences according to SEQ ID NO: 1 and SEQ ID NO: 3 are preferably the amino acid sequence of a glycoprotein, in particular a protein component or a protein moiety of a glycoprotein, of a lymphocytic choriomeningitis virus mutant. The amino acid sequence according to SEQ ID NO: 5 is preferably the amino acid sequence of an L-protein of a lymphocytic choriomeningitis virus mutant. The amino acid sequence of SEQ ID NO: 7 is preferably the amino acid sequence of an L-protein of a wild type lymphocytic choriomeningitis virus.

The invention also relates an protein or peptide, which is preferably an isolated protein or peptide, in particular a nucleoprotein or a Z-protein, comprising or consisting of an amino acid sequence according to SEQ ID NO: 12, SEQ ID NO: 20, SEQ ID NO: 28, SEQ ID NO: 36, SEQ ID NO: 44, SEQ ID NO: 52, SEQ ID NO: 60, SEQ ID NO: 14, SEQ ID NO: 22, SEQ ID NO: 30, SEQ ID NO: 38, SEQ ID NO: 46, SEQ ID NO: 54, or SEQ ID NO: 62. The invention also relates to an isolated protein or peptide having at least at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity to SEQ ID NO: 12, SEQ ID NO: 20, SEQ ID NO: 28, SEQ ID NO: 36, SEQ ID NO: 44, SEQ ID NO: 52, SEQ ID NO: 60, SEQ ID NO: 14, SEQ ID NO: 22, SEQ ID NO: 30, SEQ ID NO: 38, SEQ ID NO: 46, SEQ ID NO: 54, or SEQ ID NO: 62.

With regard to other features and advantages of the protein or peptide, in order to avoid repetitions, full reference is also made to the previous description, in particular to the statements made in the second and third aspects of the invention. The advantages and features described therein, in particular with respect to the protein or peptide, in particular glycoprotein, also apply mutatis mutandis to the isolated protein or peptide according to the fifth aspect of the invention.

According to a sixth aspect, the invention relates to an nucleic acid, which is preferably an isolated nucleic acid, in particular ribonucleic acid, comprising or consisting of a nucleic acid sequence, in particular ribonucleic acid sequence, according to or complementary to SEQ ID NO: 2, SEQ ID NO: 4, SEQ ID NO:6, SEQ ID NO:8 SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 23, SEQ ID NO: 31, SEQ ID NO: 39, SEQ ID NO: 47, SEQ ID NO: 55, or SEQ ID NO: 63. The invention also relates to an isolated protein or peptide having at least at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least about 99.5%, preferably at least about 99.6%, preferably at least about 99.7% sequence identity to SEQ ID NO: 2, SEQ ID NO: 4, SEQ ID NO: 6, SEQ ID NO: 8 SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 23, SEQ ID NO: 31, SEQ ID NO: 39, SEQ ID NO: 47, SEQ ID NO: 55, or SEQ ID NO: 63 or a respective complementary sequence.

The invention also relates to a nucleic acid, which is preferably an isolated nucleic acid, in particular ribonucleic acid, comprising or consisting of a nucleic acid sequence, in particular ribonucleic acid sequence, according to or complementary to SEQ ID NO: 11, SEQ ID NO: 19, SEQ ID NO: 27, SEQ ID NO: 35, SEQ ID NO: 43, SEQ ID NO: 51, SEQ ID NO: 59, SEQ ID NO: 13, SEQ ID NO: 21, SEQ ID NO: 29, SEQ ID NO: 37, SEQ ID NO: 45, SEQ ID NO: 53, or SEQ ID NO: 61. The invention also relates to an isolated protein or peptide having at least at least about 95%, preferably at least about 96%, preferably at least about 97%, preferably at least about 98% preferably at least about 99%, preferably at least about 99.1%, preferably at least about 99.2%, preferably at least about 99.3%, preferably at least about 99.4%, preferably at least SEQ ID NO: 11, SEQ ID NO: 19, SEQ ID NO: 27, SEQ ID NO: 35, SEQ ID NO: 43, SEQ ID NO: 51, SEQ ID NO: 59, SEQ ID NO: 13, SEQ ID NO: 21, SEQ ID NO: 29, SEQ ID NO: 37, SEQ ID NO: 45, SEQ ID NO: 53, or SEQ ID NO: 61 or a respective complementary sequence.

The nucleic acid sequences according to SEQ ID NO: 2 and SEQ ID NO: 4 each preferably encode for a glycoprotein, in particular for a protein component or a protein moiety of a glycoprotein, of a lymphocytic choriomeningitis virus mutant.

The nucleic acid sequence according to SEQ ID NO: 6 preferably encodes for an L-protein of a lymphocytic choriomeningitis virus mutant.

The nucleic acid sequence of SEQ ID NO: 8 preferably encodes for an L-protein of a wild type lymphocytic choriomeningitis virus.

With regard to further features and advantages of the isolated nucleic acid, in order to avoid repetitions, full reference is also made to the previous description, in particular to the statements made in the context of the second and third aspects of the invention. The features and advantages described therein, in particular with regard to nucleic acid, in particular ribonucleic acid, also apply mutatis mutandis to the isolated nucleic acid according to the sixth aspect of the invention.

According to a seventh aspect, the invention relates to an isolated gene cluster or operon containing at least one nucleic acid, in particular ribonucleic acid, according to the fifth aspect of the invention.

With regard to further features and advantages of the gene cluster or operon, in order to avoid repetitions, full reference is also made to the previous description, in particular to the statements made in the context of the sixth aspect of the invention. The features and advantages described therein, in particular with regard to nucleic acid, in particular ribonucleic acid, also apply mutatis mutandis to the isolated gene cluster or operon according to the seventh aspect of the invention.

According to an eighth aspect, the invention relates to an expression vector containing at least one nucleic acid according to the sixth aspect of the invention or a gene cluster or operon according to the seventh aspect of the invention.

With regard to further features and advantages of the expression vector, in order to avoid repetitions, full reference is also made to the description, in particular to the statements made under the sixth and seventh aspects of the invention. The features and advantages described therein, in particular with respect to nucleic acid and gene cluster or operon, also apply mutatis mutandis to the expression vector according to the eighth aspect of the invention.

According to a ninth aspect, the invention relates to an organism, a virus, a vector or a plasmid which expresses or contains a protein or peptide according to the fifth aspect of the invention and/or which contains a nucleic acid, in particular ribonucleic acid, according to the sixth aspect of the invention.

The organism can be a host cell, a fungus or a bacterium.

The host cell can, for example, be a eukaryotic or prokaryotic cell. Furthermore, the host cell can be a cell that can be used for the production of organisms and/or vectors and/or plasmids.

The bacterium can for example be a vaccine vector or another therapeutic bacterium.

The virus can for example be a vaccine vector or another therapeutic virus.

With regard to further features and advantages of the host cell, in order to avoid repetitions, full reference is also made to the description, in particular to the statements made in the fifth and sixth aspects of the invention. The features and advantages described there, in particular with regard to the protein or peptide, in particular glycoprotein, and the nucleic acid, in particular ribonucleic acid, also apply mutatis mutandis to the host cell according to the ninth aspect of the invention.

Further features and advantages of the invention result from the following description of preferred embodiments in the form of examples, the corresponding figures and the claims. The embodiments described in the following are only intended to provide further description and a better understanding of the invention and should by no means be construed as limiting.

The present invention also relates to a pharmaceutical composition comprising a LCMV of the disclosure, preferably a LCMV mutant of the disclosure. The composition may further comprise pharmaceutically acceptable excipient. The pharmaceutical composition can further comprise a checkpoint blocker, such as PD-1 (Programmed Cell Death Protein 1), and/or an apoptosis modulator, in particular an apoptosis inhibitor, such as SMAC-mimeticum (LCL-161). The pharmaceutical composition may further comprises a pharmaceutically acceptable carrier. The carrier may be selected from the group consisting of water, aqueous saline solution, aqueous buffer solution, cell culture medium and combinations of at least two of the foregoing carriers.

The present disclosure also relates to the use of a LCMV of the disclosure, preferably a LCMV mutant of the disclosure for the manufacture of a medicament. The medicament may be for the treatment a tumor. The medicament may be a medicament according to the fourth aspect of the invention.

The present disclosure also relates to a method of treating a tumor, comprising administering to a subject in need thereof an effective amount of an LCMV of the disclosure, preferably an LCMV mutant of the disclosure, or a medicament of the disclosure or a pharmaceutical composition of the disclosure.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms. E.g. the term “comprising” is meant to provide explicit support also for “consisting essentially of” and “consisting of”, the term “consisting essentially of” is meant to provide explicit support also for “comprising” and “consisting of”, the term “consisting of” is meant to provide explicit support also for “consisting essentially of” and “comprising”,

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

### 1. Methods and Materials

### 1.1 Mice

For in vivo anti-tumoral analyses WT animals (on C57BL/6 background) or NOD.SCID mice without an adaptive immune systrain WEre used. OT-1 mice carry an ovalbumin-specific MHC-I restricted T cell receptor as transgene.

### 1.2 Cell Lines

MC57 (CVCL_4985) is a murine fibroblast cell line in which the arenavirus LCMV can multiply well. C643 (CVCL_5969) is a human anaplastic thyroid carcinoma cell line. H1975 is a human lung carcinoma cell line (CVCL_1511, ATCC, CRL-5908, adenocarcinoma). TrampC2 is a murine adenocarcinoma cell line (CVCL_3615). MOPC is a murine oropharyngeal cell line. CMT167(CVCL_2405) is a murine lung carcinoma cell line. B16F10-OVA is a murine melanoma cell line (CVCL_0159) expressing ovalbumin as a model antigen. UKE-Mel-13a are primary tumor cells isolated from a human melanoma metastasis and have been passeged less than 100×. 511950 are primary tumor cells isolated from a transgenic murine pancreatic carcinoma (Mazur P K et al Nat Med. 2015 October; 21(10):1163-71.) and have been passaged less than 20×. 511950R originate from 511950 cells and have been passaged less than 100× under treatment with the MEK inhibitor trametinib.

### 1.3 Viruses

The LCMV strain WE was obtained from the laboratory of Prof. Zinkemagel (Experimental Immunology, Zurich, Switzerland) and has been propagated in L929 cells or BHK cells since 2008. The clones LCMV-P42, LCMV-P52 and LCMV-P91 were isolated and sequenced after different passages.

### 1.4 Reagents

LCL161 (Selleckchem) and anti-PD1 (BioXcell) were tested in combination with LCMV and LCMV-P52, respectively, for their anti-tumor activity.

### 1.5 Determination of LCMV Infected Cells by Immunofluorescence

Immunofluorescence was used to detect LCMV in cells. Cells were seeded in 24 well plates, each containing a cover glass. After 24 hours the cells were infected with LCMV and stained 24 hours later with a fluorochrome-labelled anti-LCMV-NP antibody (clone VIA), visualized with a fluorescence microscope and photographed with an integrated CCD camera.

### 1.6 Determination of LCMV in Supernatant by Plaque Assay

To determine the LCMV production of cells, cells were sown in 24-well plates and infected with LCMV after 24 hours. The supernatant was extracted after 24 hours. The supernatant was titrated in 24-well plates and MC57 cells (150,000 cells/hole) were added. Methyl cellulose was added after 4 hours. After another 48 hours, the cell lawn was analyzed for LCMV plaques with anti-LCMV-NP antibodies (clone KL53). The plaques were counted to determine the number of infectious particles/ml in the supernatant.

### 1.7 Passages of Viruses

In order to adapt viruses to tumors, different primary tumor cells or tumor cell lines were infected with LCMV-WE. Cells were plated in 24-well plates (approx. 100,000 cells/well in 1 mL medium). After 24 hours viruses with a Multiplicity of Infection (MOI)=1 in 100 μL were added. Depending on the setup, the initial inoculum was removed between 1-30 minutes and new medium was added. After 24 or 48 or 72 hours the cell culture supernatant was extracted and frozen for further analysis. Newly plated cells were infected with 100 microL of the extracted supernatant. This procedure was repeated between 30 and 100 times.

### 1.8 Sequencing

After reverse transcription of the viral RNA, the cDNA was amplified with sequence specific primer pairs (oligonucleotides) in the polymerase chain reaction (PCR). The PCR products were purified, sequenced by cycle sequencing (modified singer method), the products separated by capillary electrophoresis and the sequence recorded as an electropherogram. The nucleic acid sequence was translated to obtain the protein sequence.

### 1.9 Innate Immune Activation

The ability of LCMV to activate the innate immune system was determined by the biomarker IFN-alpha using murine IFN-alpha ELISA (ThermoFisher).

### 1.10 Adaptive Immune Activation

The ability of LCMV to activate the adaptive immune system was tested by tetramer staining (NIH, Tetramer Facility) of activated lymphocytes.

### 1.11 Tumor Growth and Treatments

To measure the anti-tumoral effect, C57BL/6 or NOD.SCID mice (6-12 weeks old) 5×10⁵ tumor cells (in 100 μL) were injected subcutaneously into the right or left flank. After a visible tumor was formed, the animals were treated and the mean tumor diameter or tumor volume was determined. For a metastasis model, B16F10-OVA cells were applied intravenously.

### 1.12 Isolation and Transfer of Ovalbumin (Tumor)-Specific CD8+ T Cells

For the analysis of tumor-specific CD8+ T cells, cells from the spleen of OT-1 mice were transferred into C57BL/6 mice carrying ovalbumin-expressing tumor (B16F10-OVA) cells. Spleens were mechanically crushed. After filtration, 10⁷ spleen cells per mouse were injected intravenously.

### 1.13 Measurement of Tumor-Specific CD8+ T Cells

To analyze the number of tumor-specific CD8+ T cells, cells from the blood were incubated with fluorescent ovalbumin tetramers (four coupled H-2 Kb MHC-I molecules carrying the peptide SIINFEKL, NIH Tetramer Facility) and fluorochrome-coupled anti-CD8 antibodies (eBioscience) and analyzed after washing in a flow cytometer. For the analysis of T cell function, spleen cells were mechanically crushed and incubated after filtration with Brefeldin A and with or without SIINFEKL peptide. After six hours the cells were fixed, permeabilized and stained with fluorescent antibodies specific for CD8 (anti-CD8, eBioscience) and intracellular interferon-gamma (anti-interferon-gamma, eBioscience). The frequency of IFN-gamma producing cells was analyzed by flow cytometry.

### 1.14 Statistical Analysis

The mean values were compared using an unpaired two-sample Student's t-test. The data are presented as mean±SEM. The level of statistical significance was determined to be p<0.05.

### 1.15 Generation of LCMV-P42:

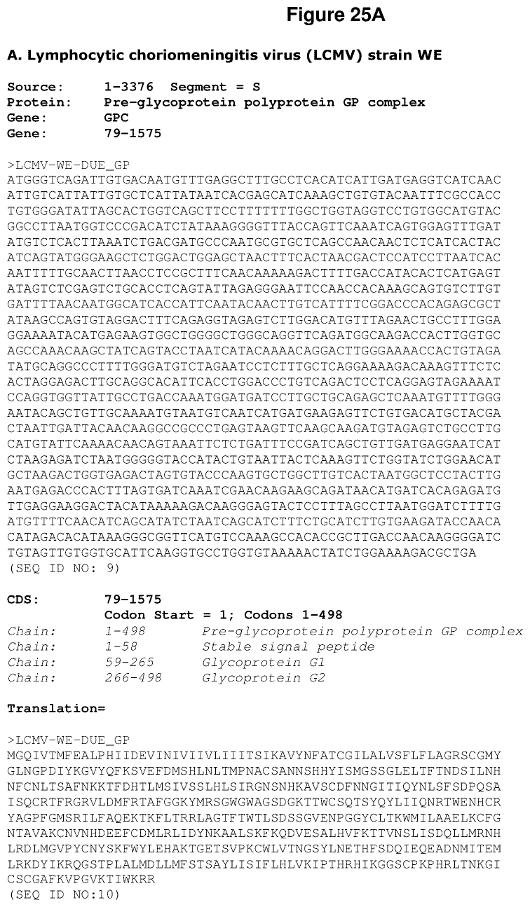

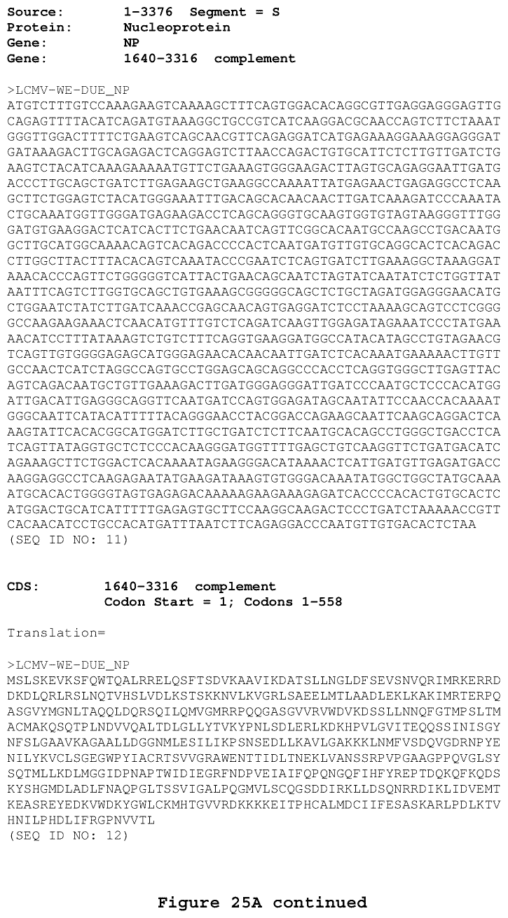

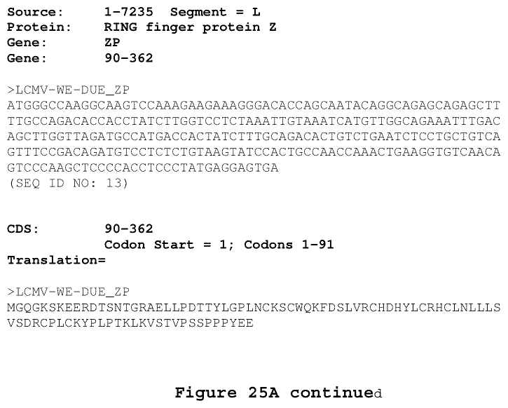

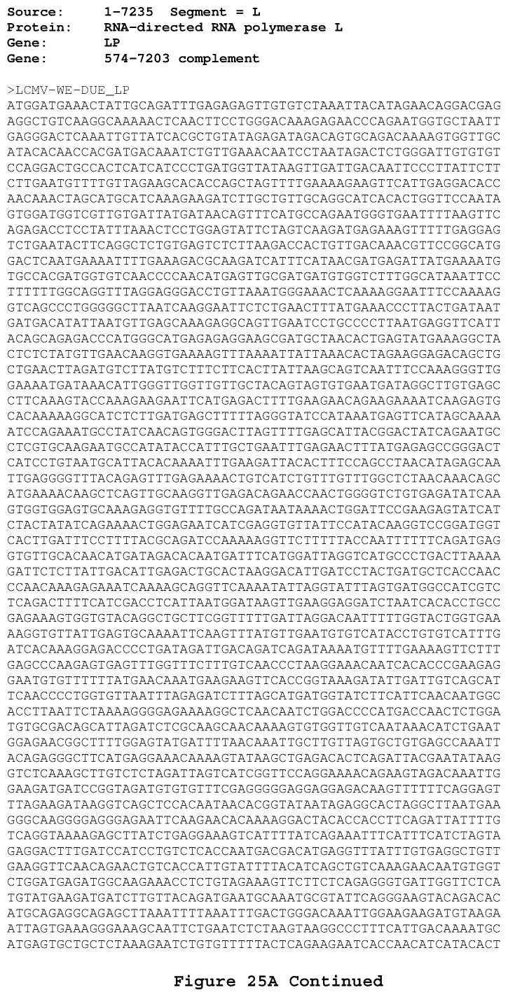

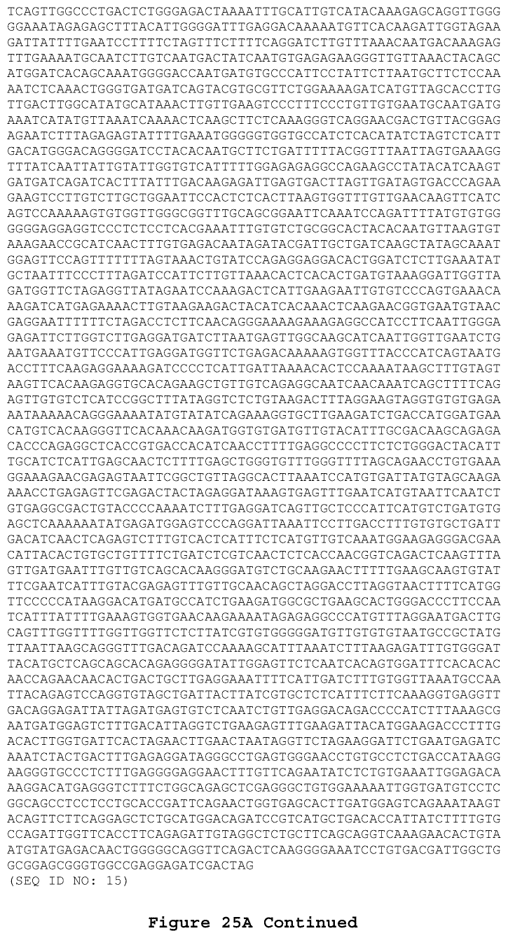

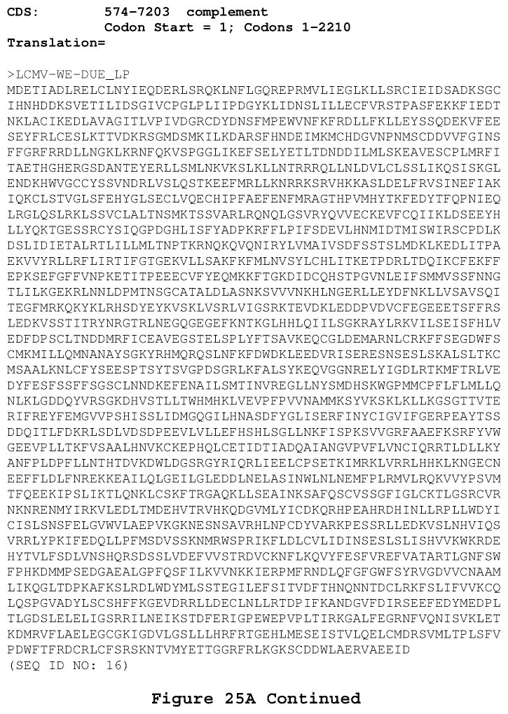

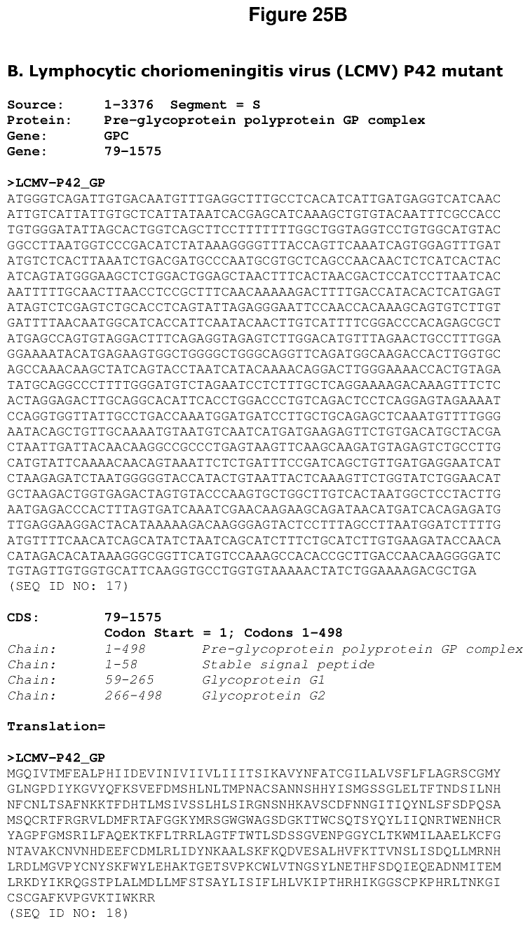

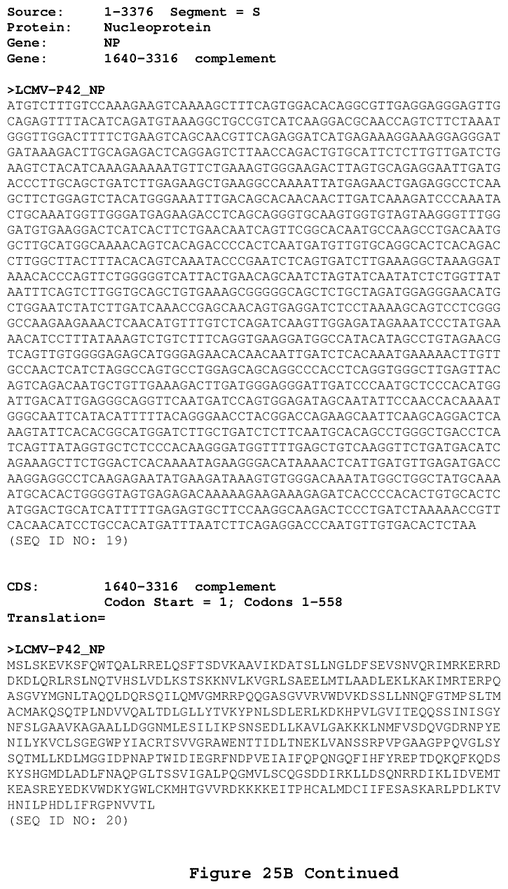

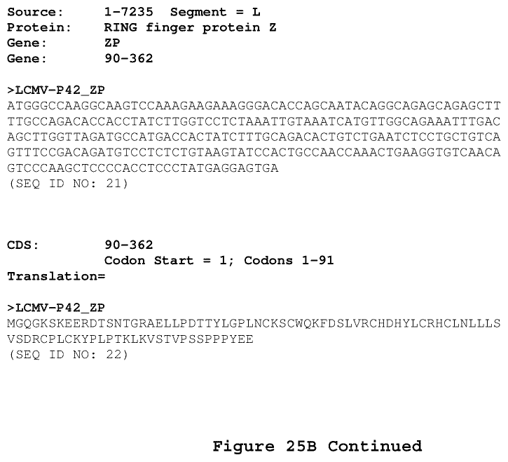

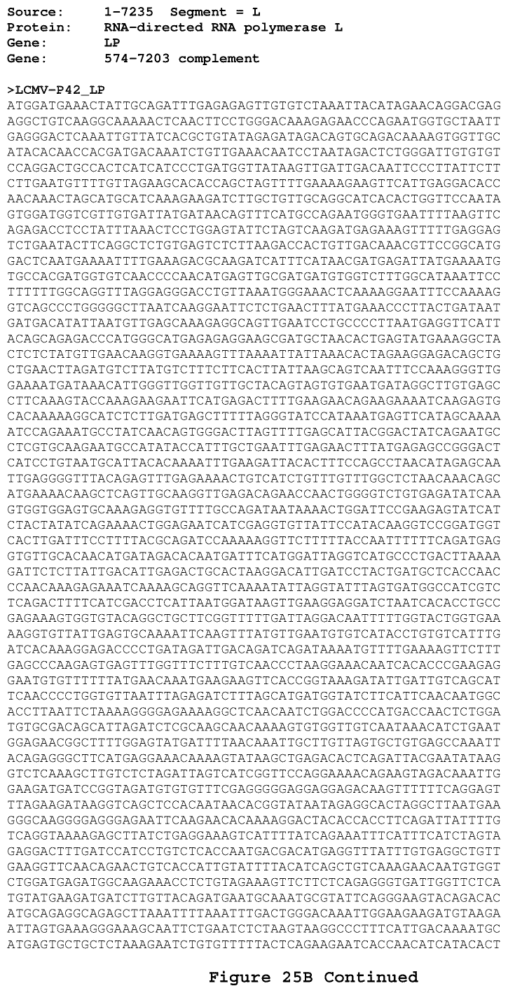

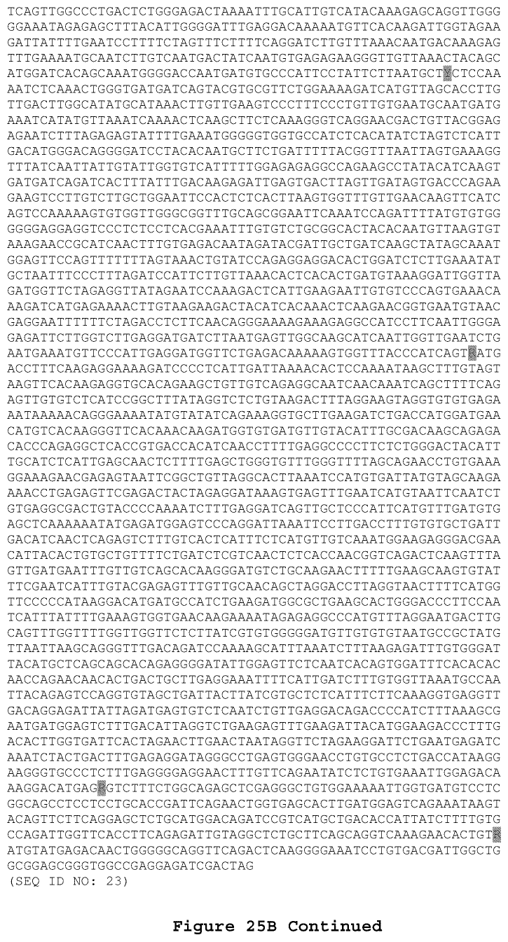

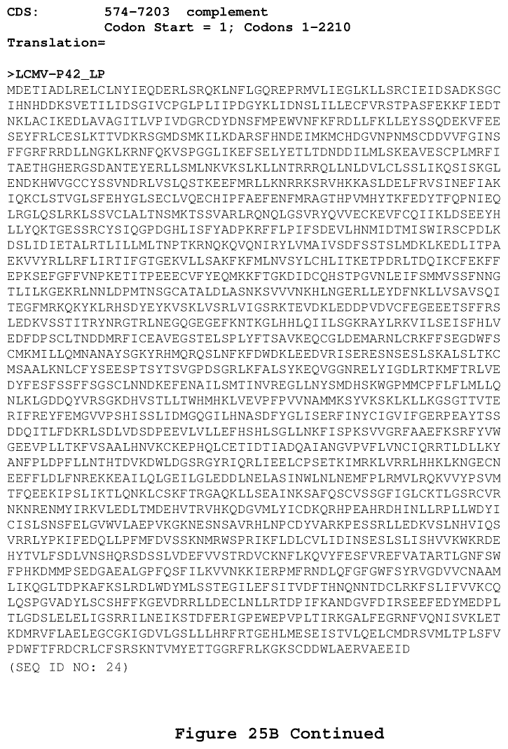

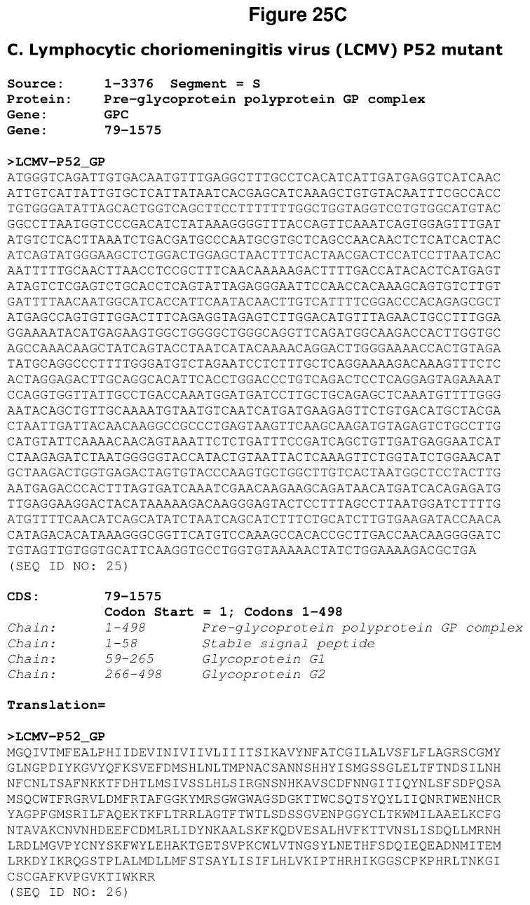

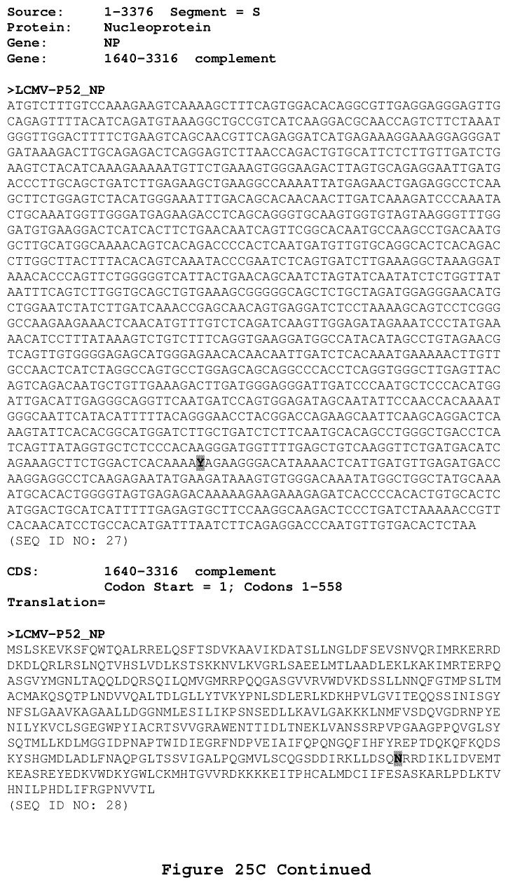

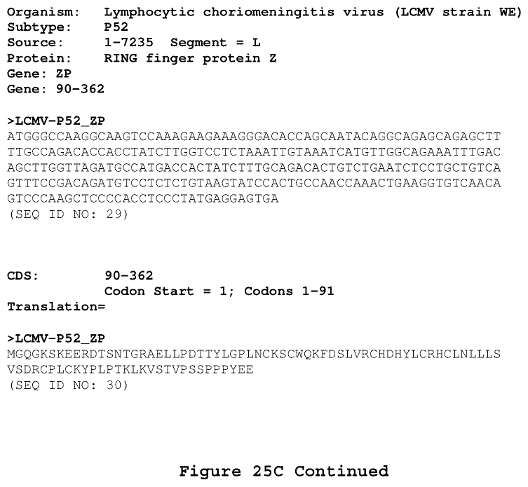

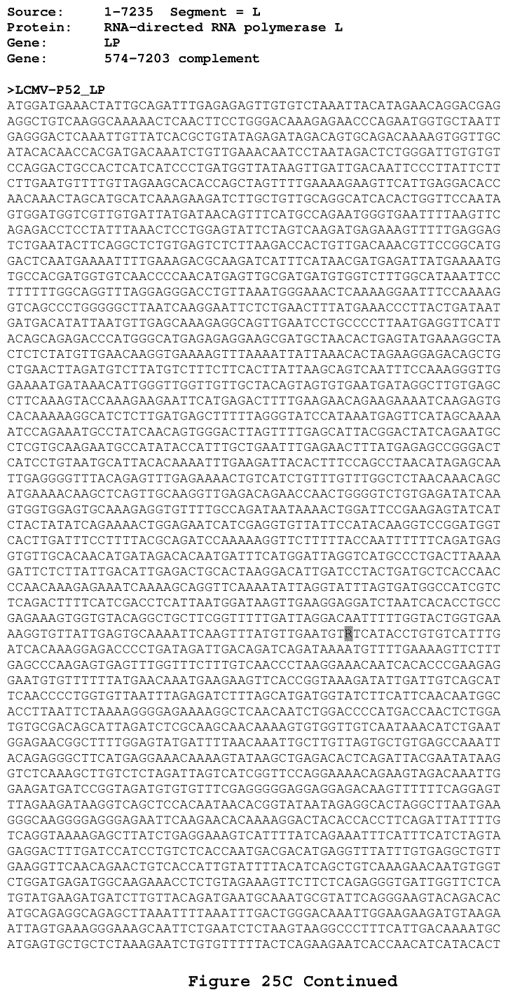

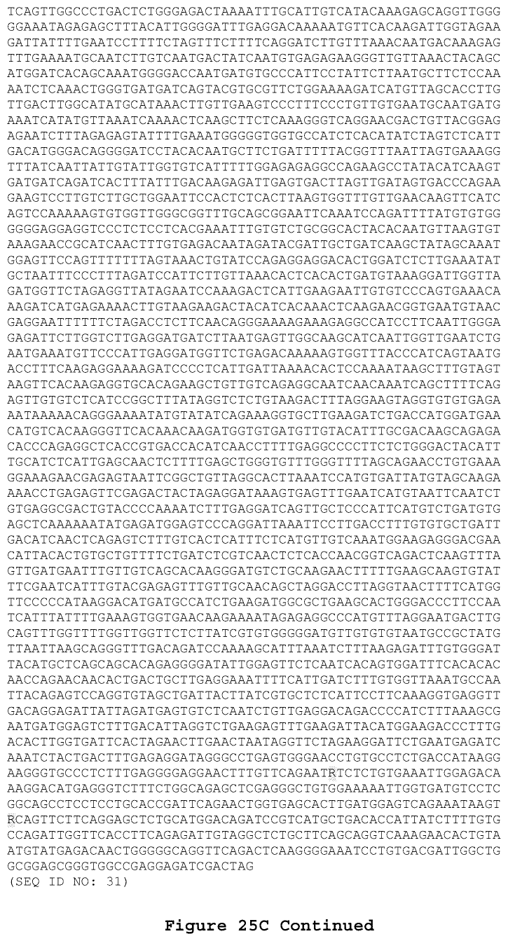

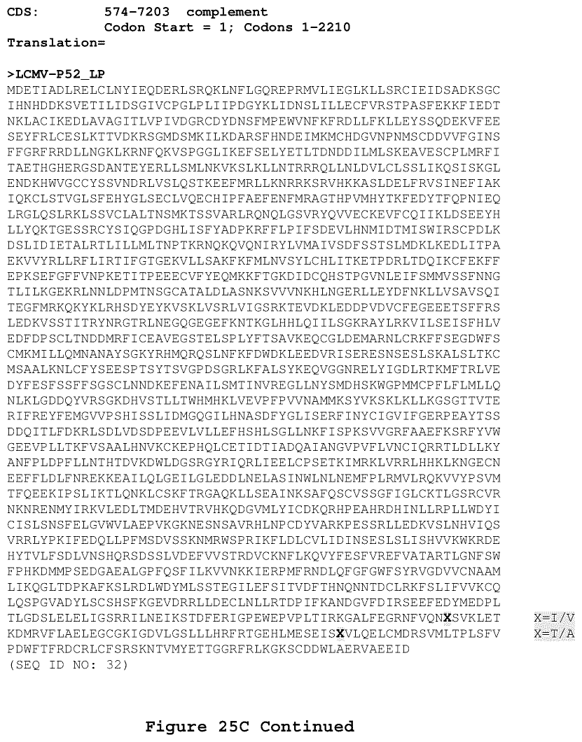

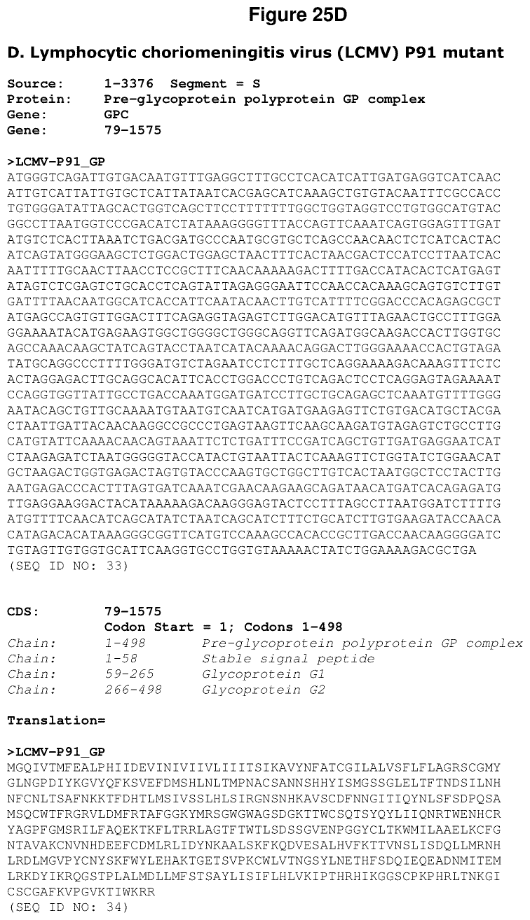

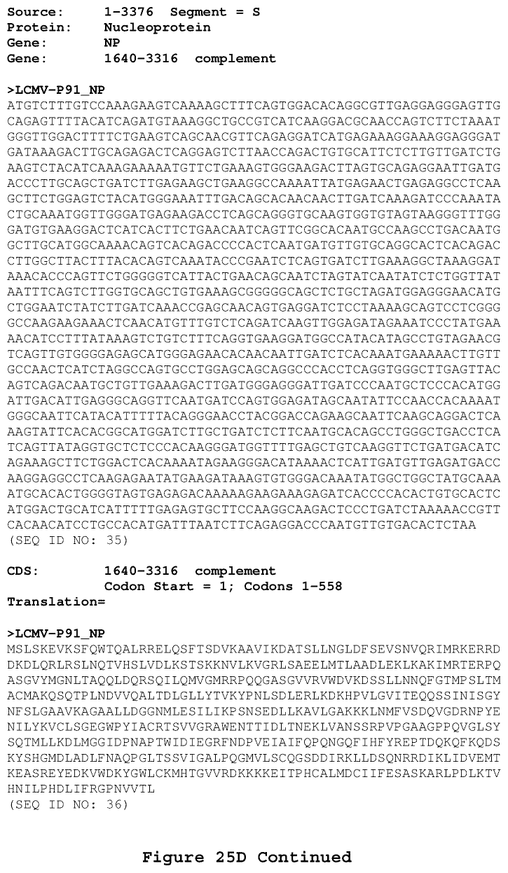

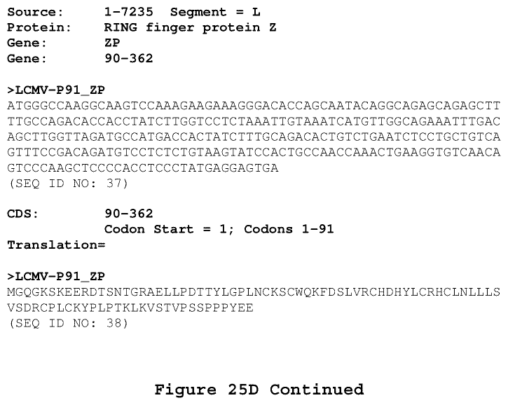

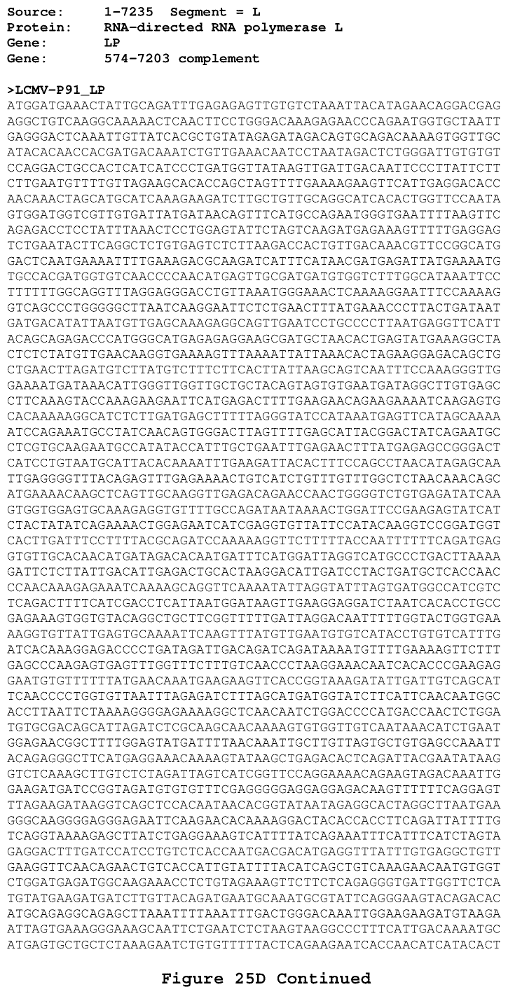

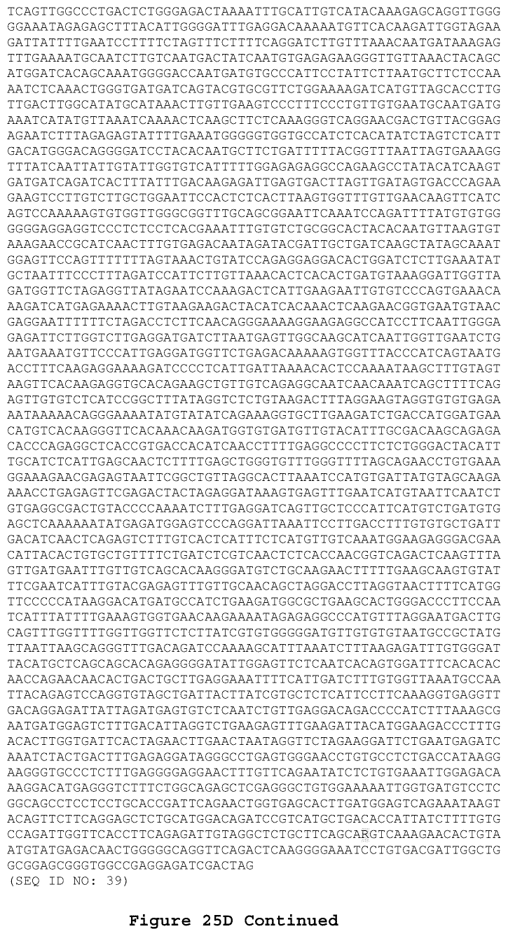

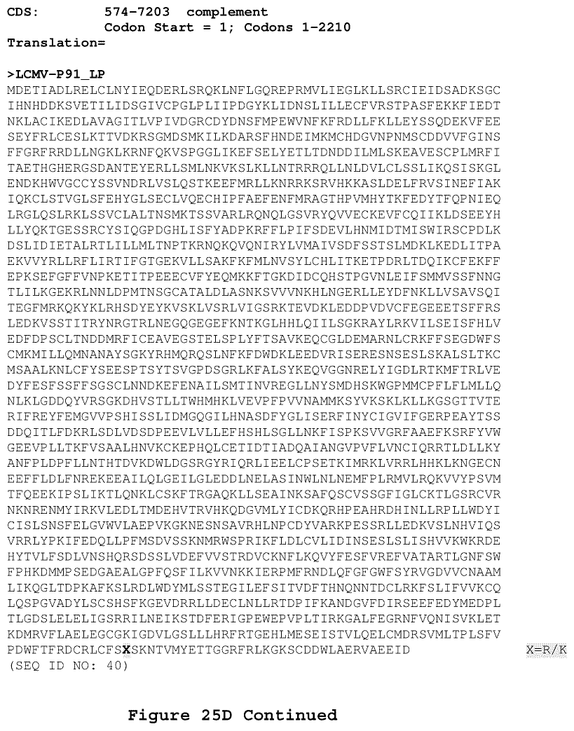

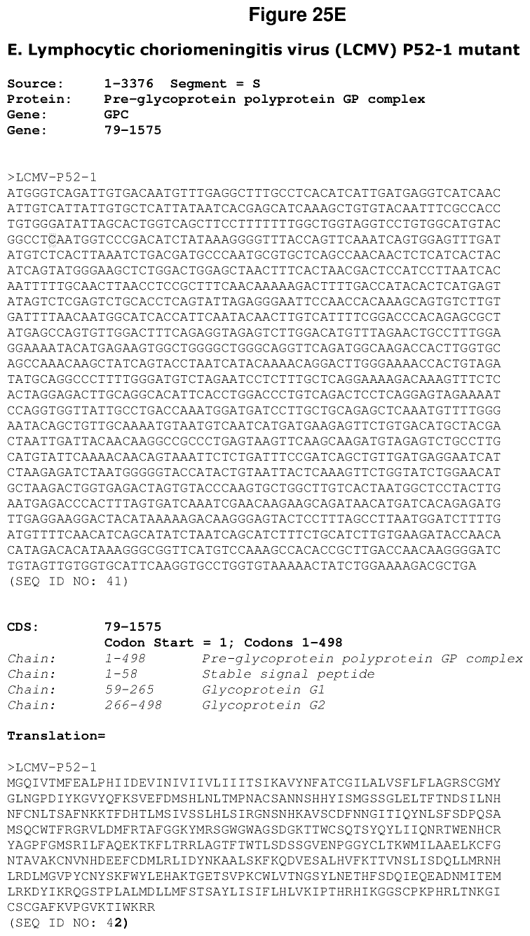

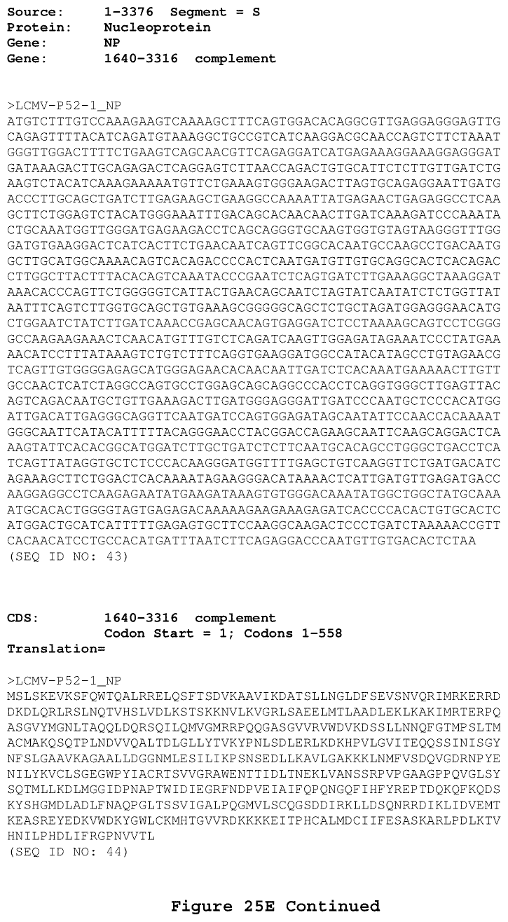

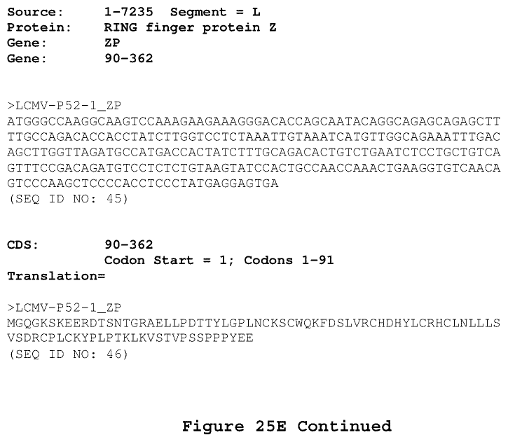

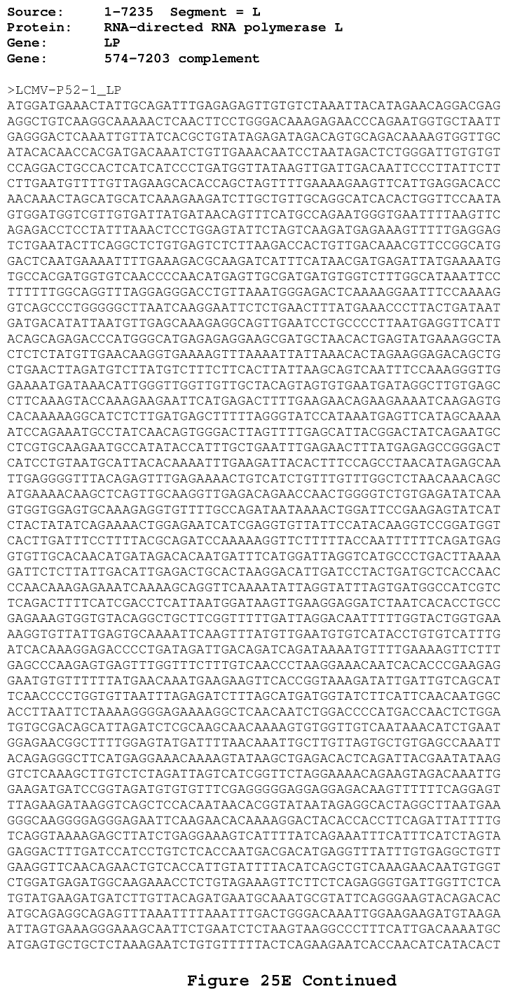

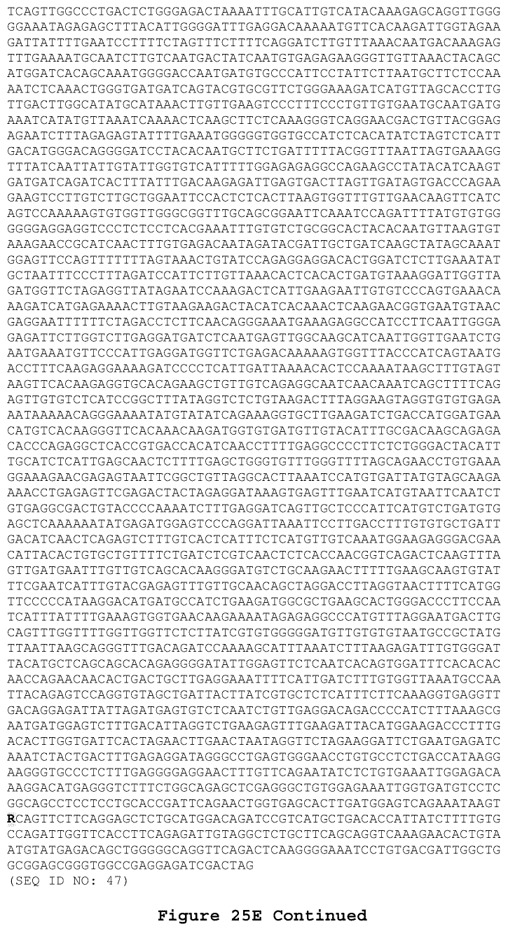

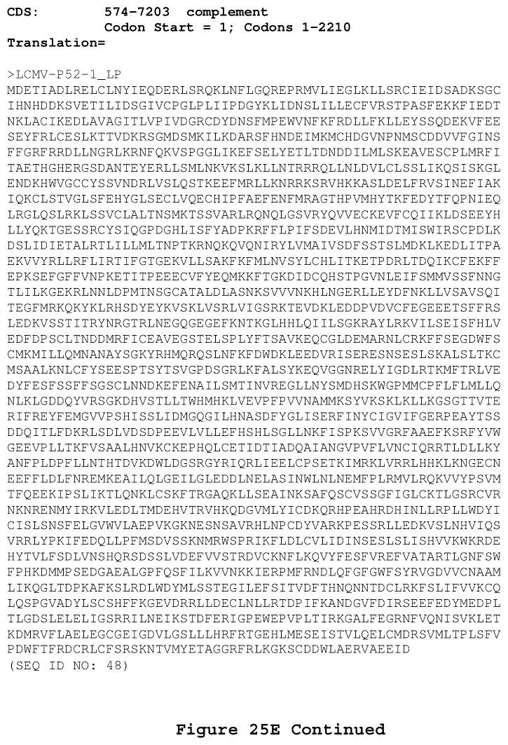

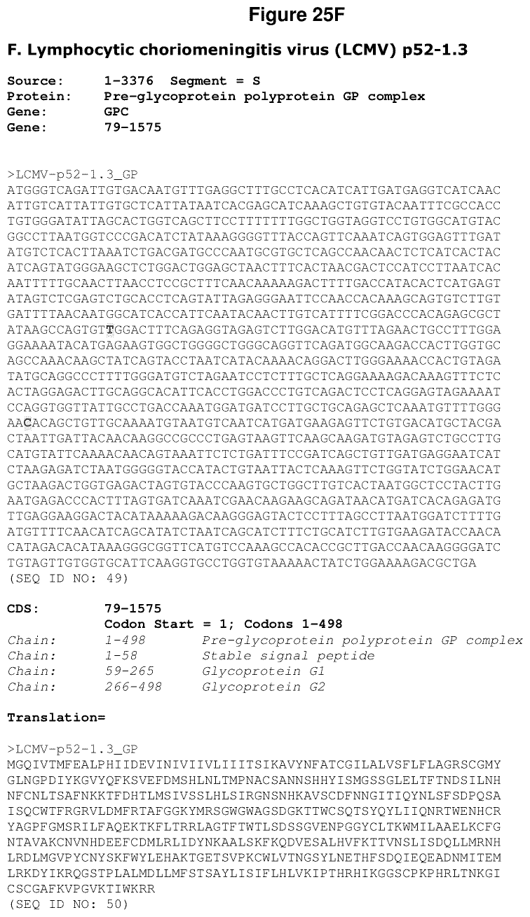

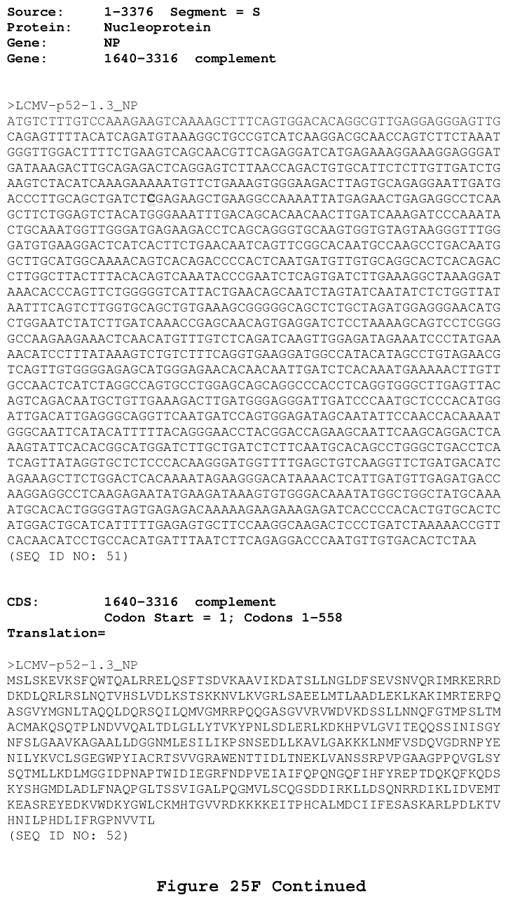

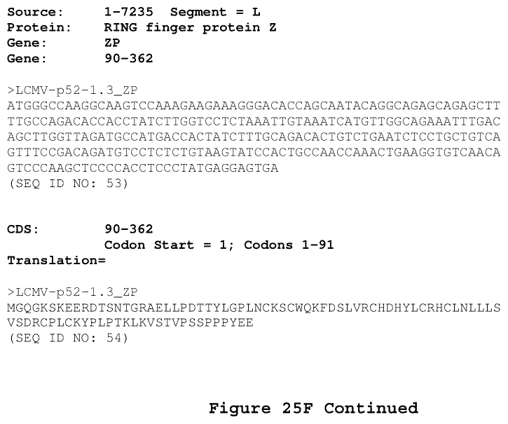

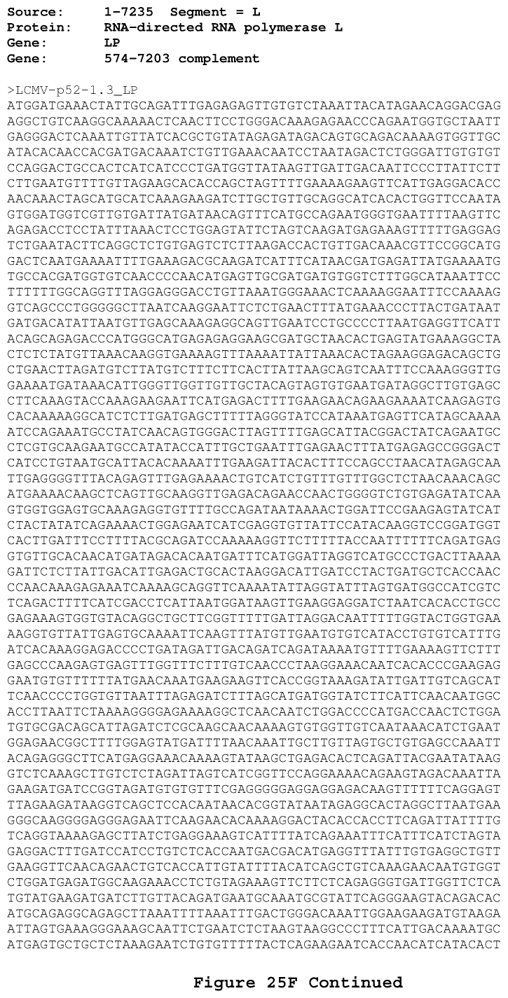

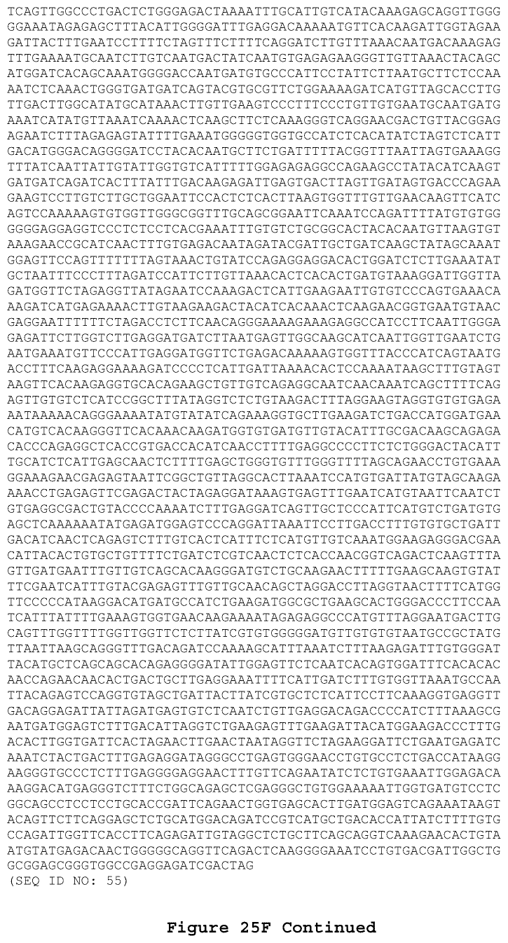

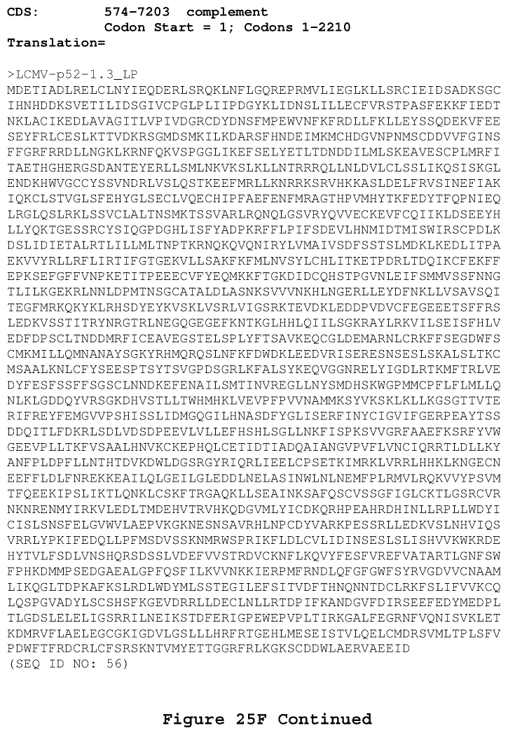

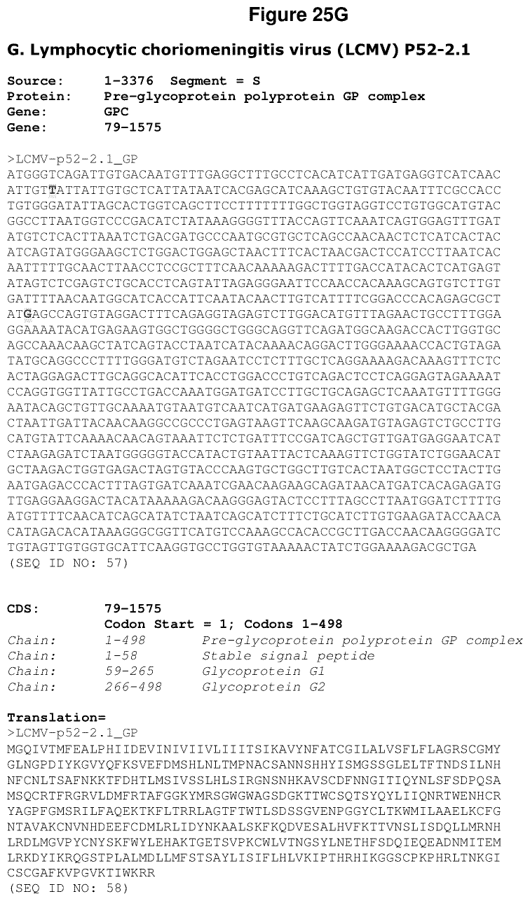

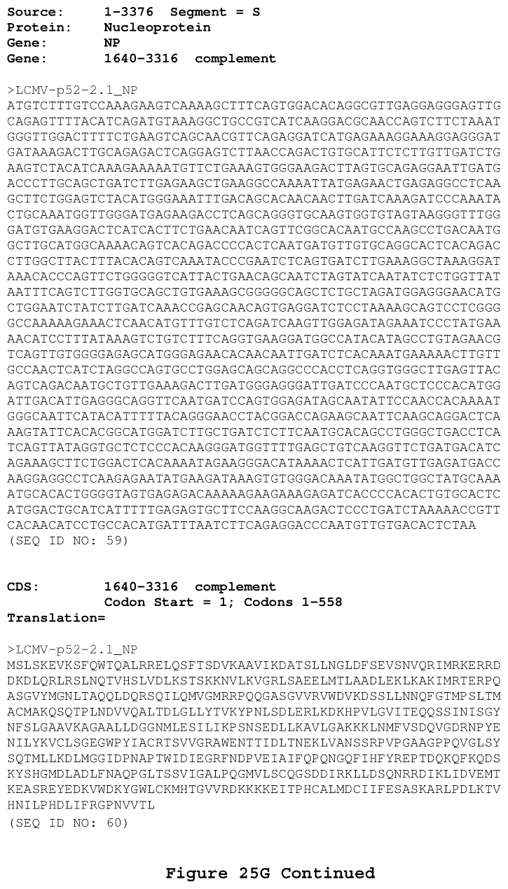

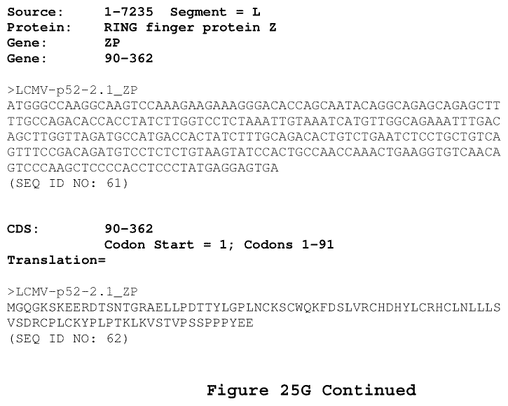

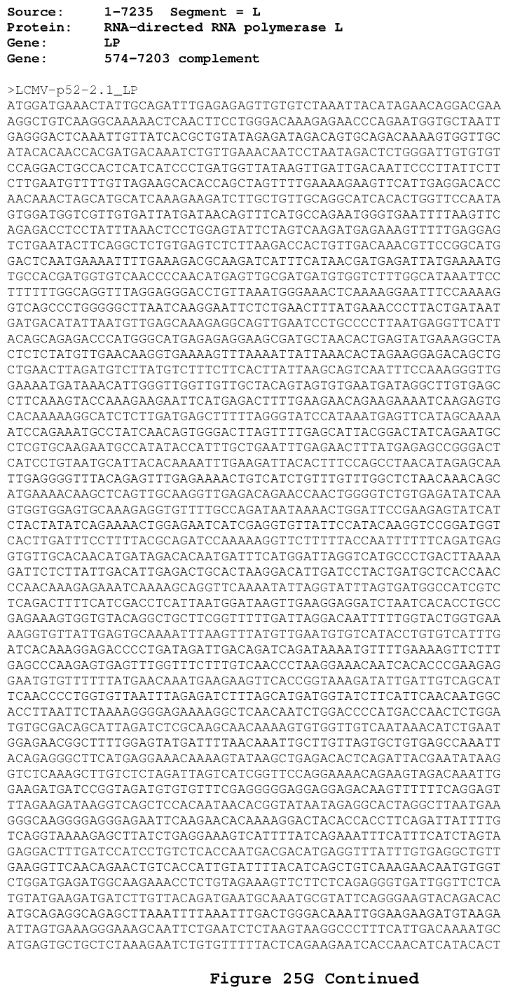

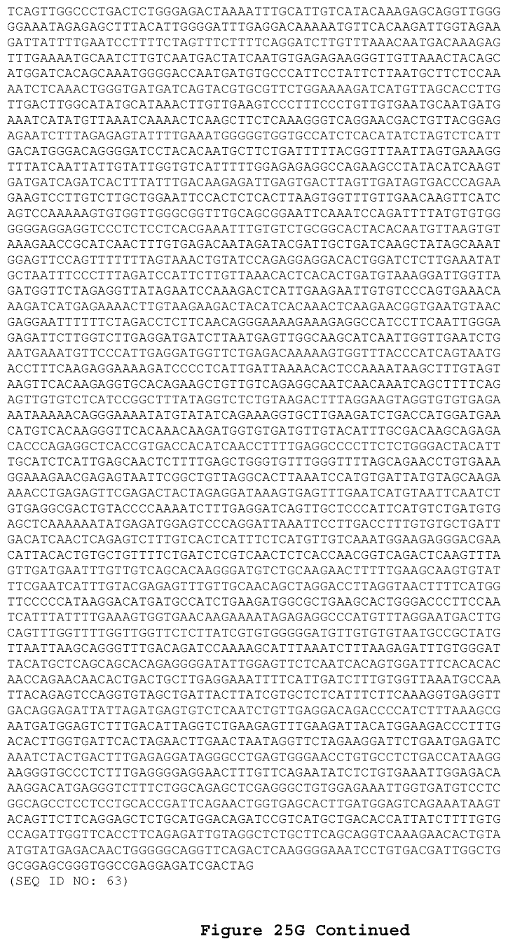

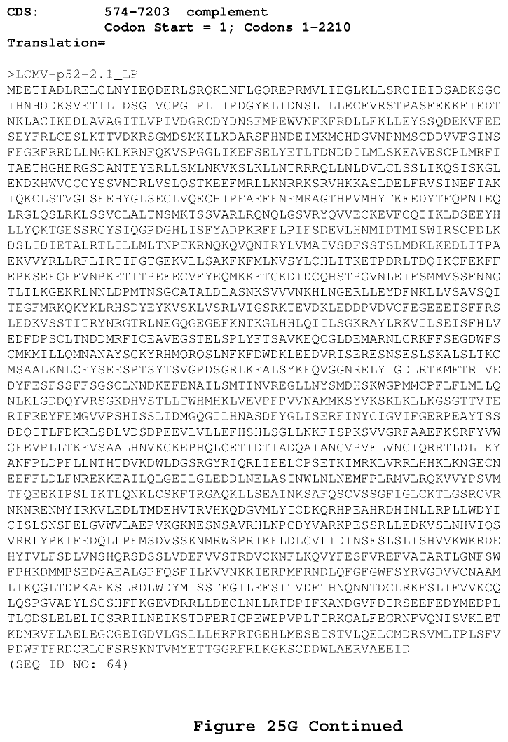

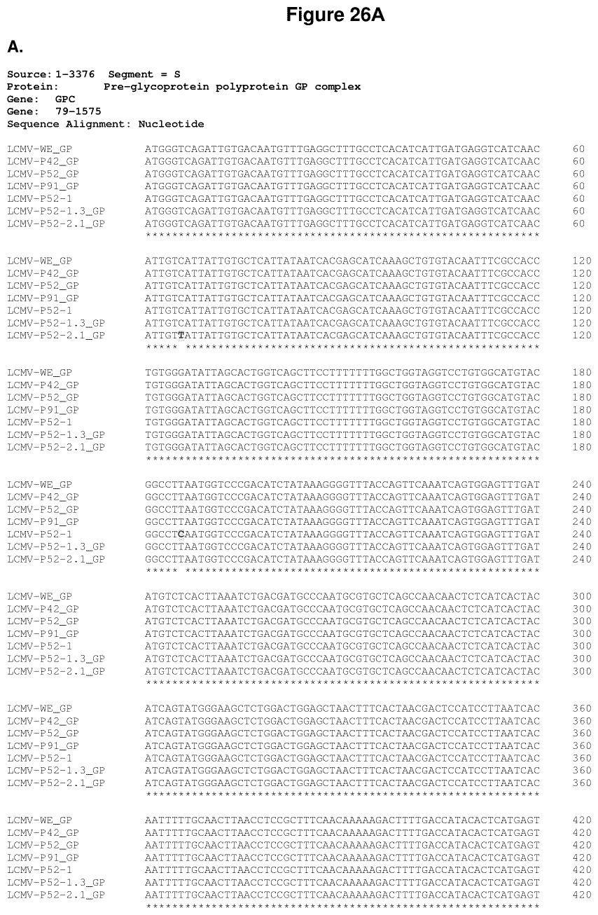

LCMV-WE was passaged 42 times in primary tumor cell cultures (UKE-Mel-13a). After 42 passages, a functional mutation was detected in the new virus (LCMV-P42). Nucleic acid and amino acid sequences of LCMV-WE and mutant P42 are shown in FIG. 25 (A and B).

### 1.16 Generation of LCMV-P91, LCMV-P52 and It's Subdones:

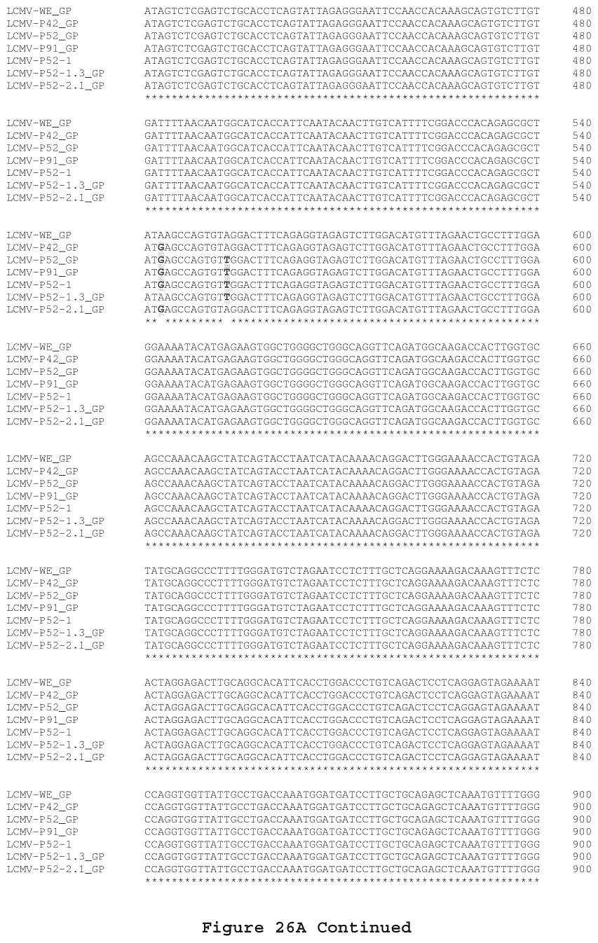

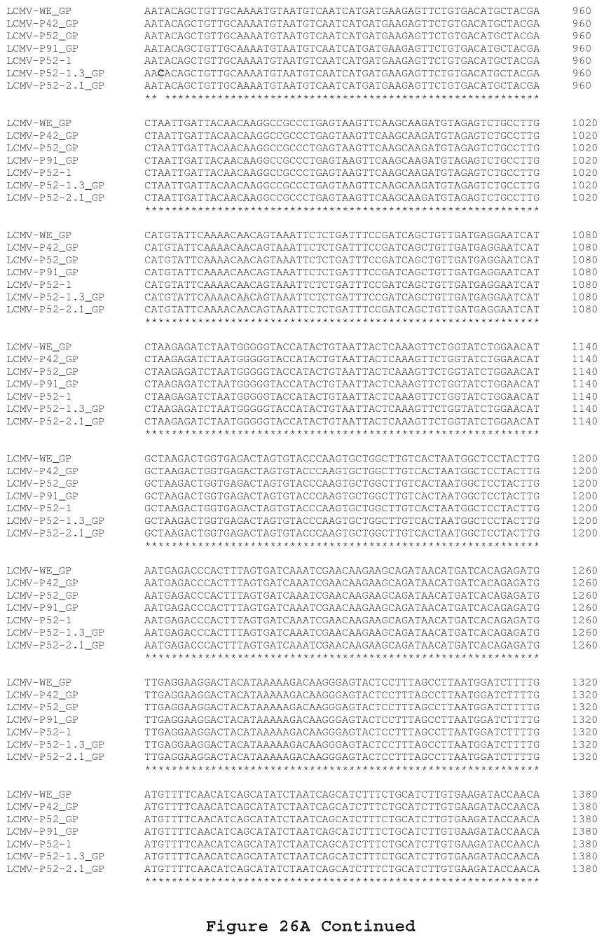

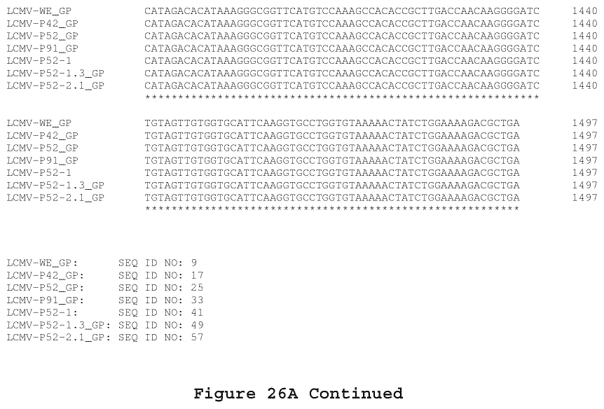

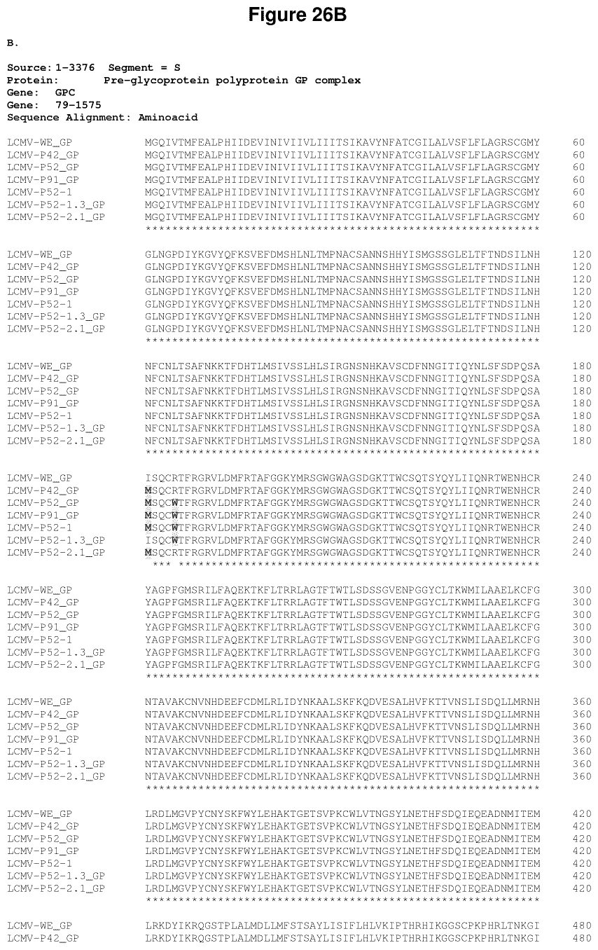

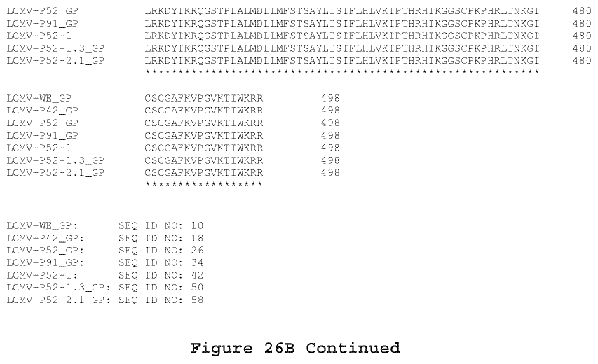

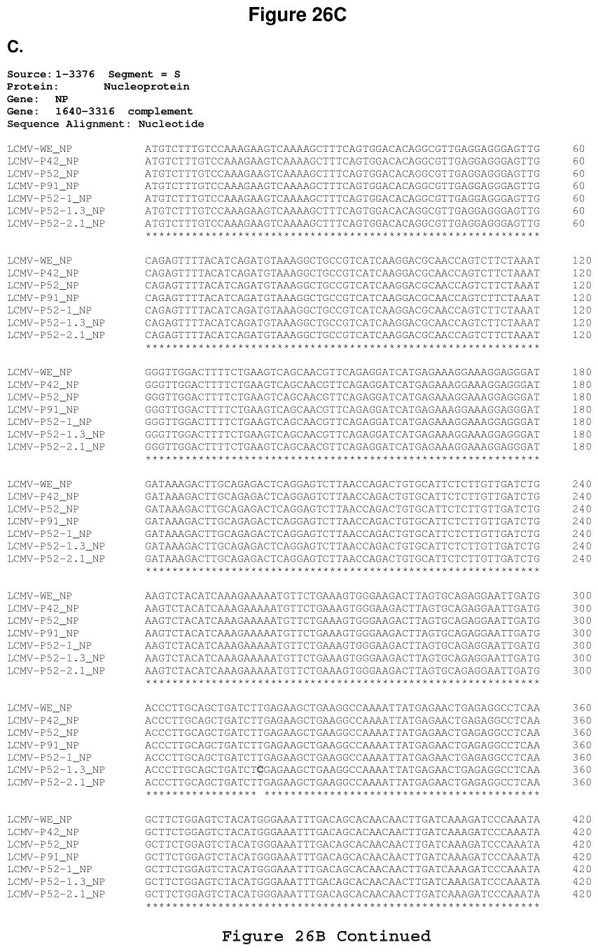

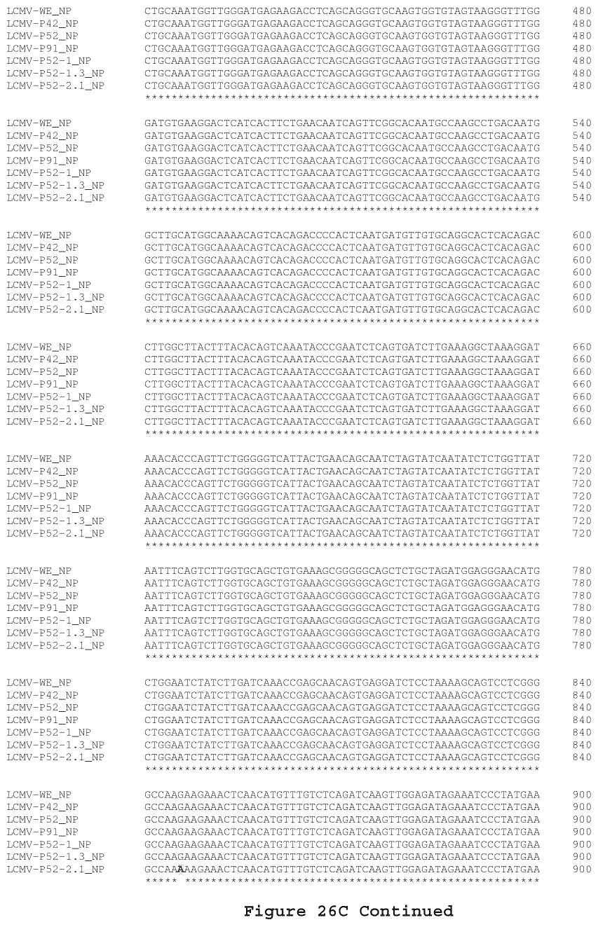

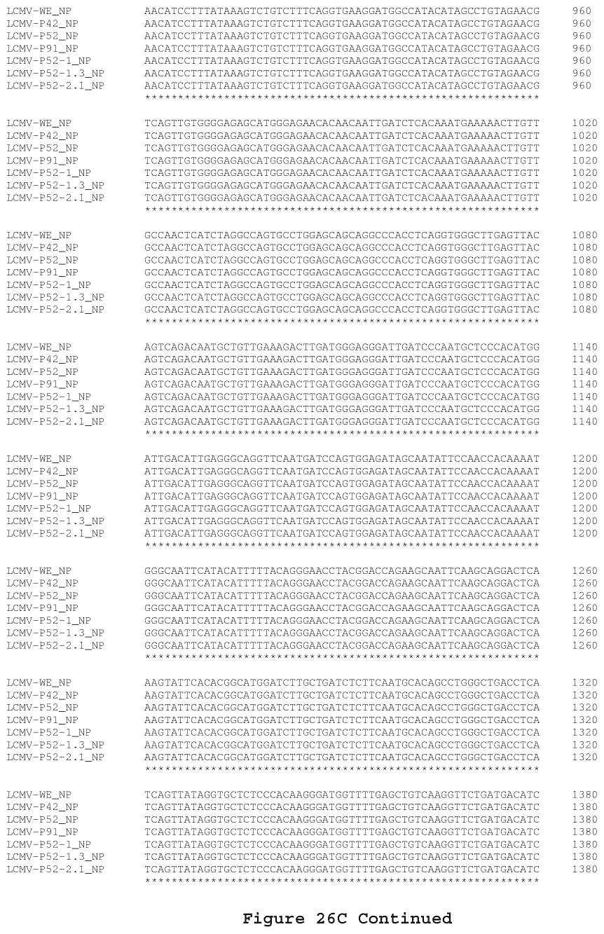

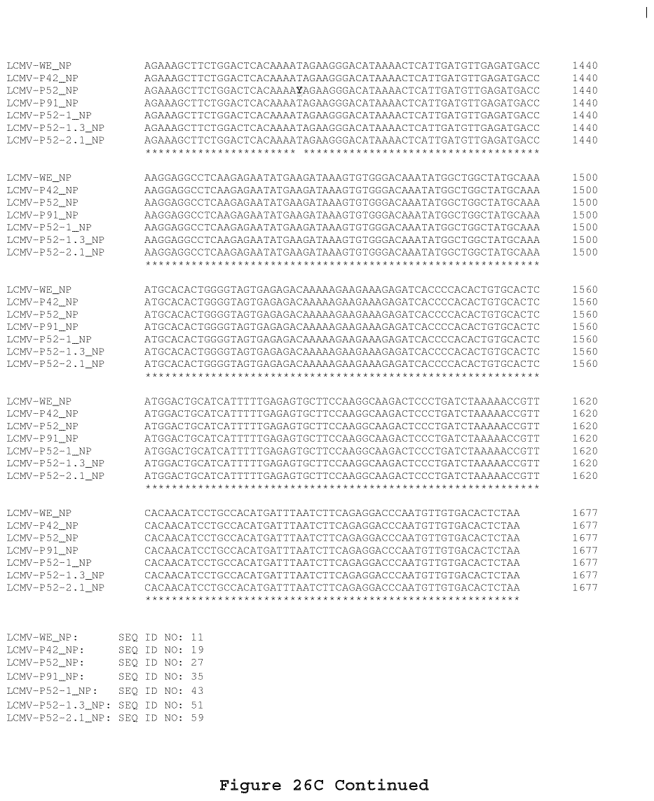

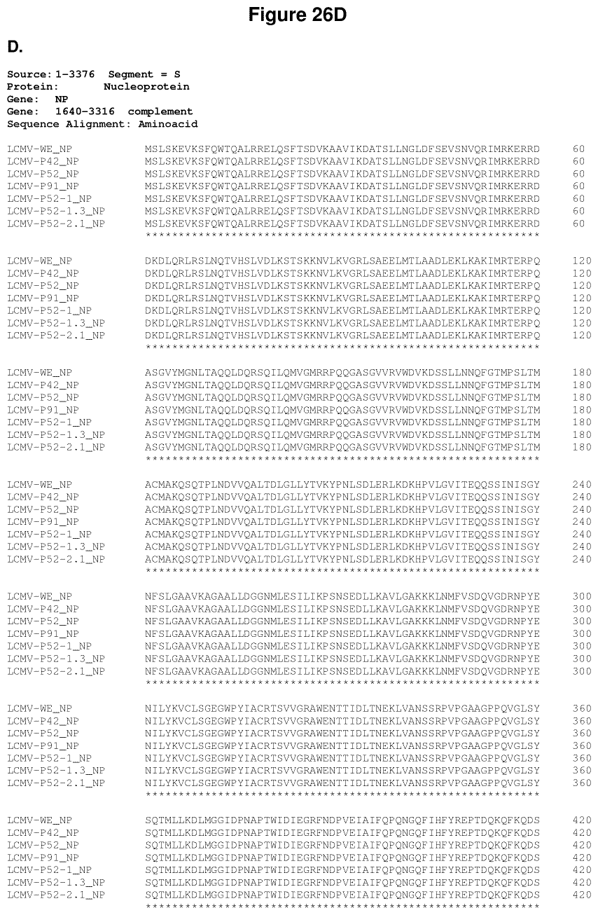

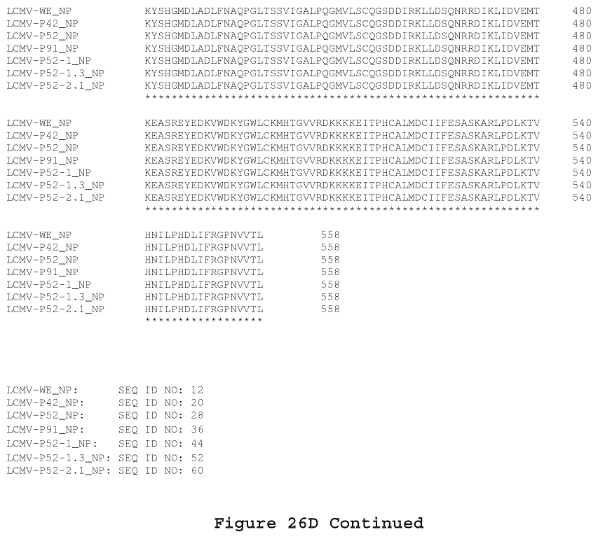

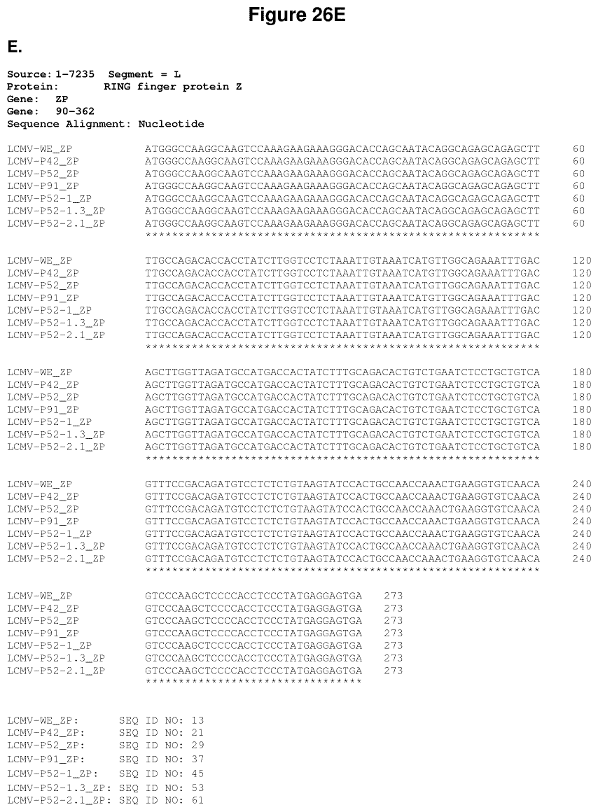

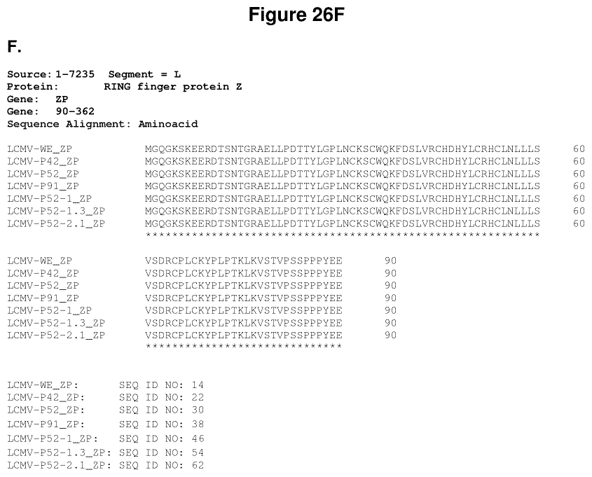

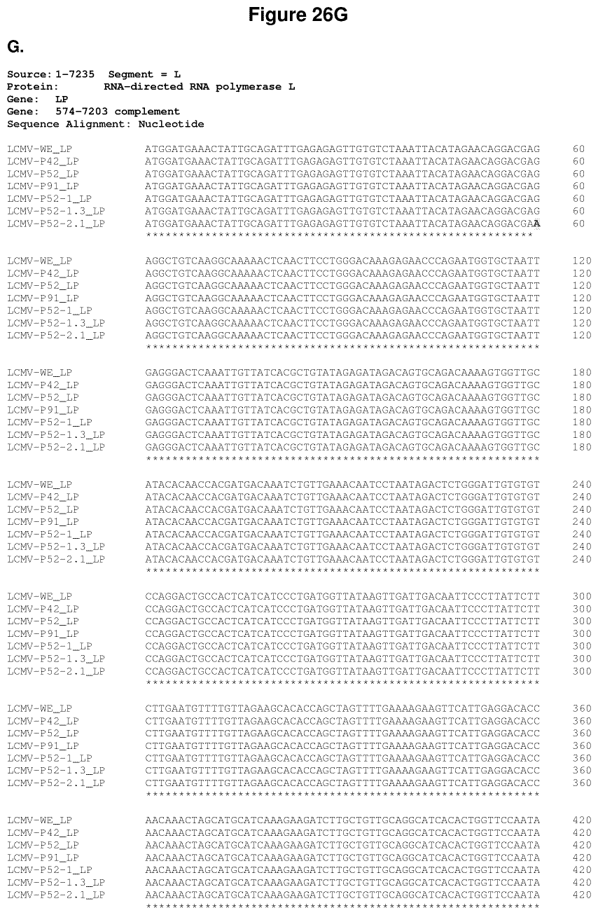

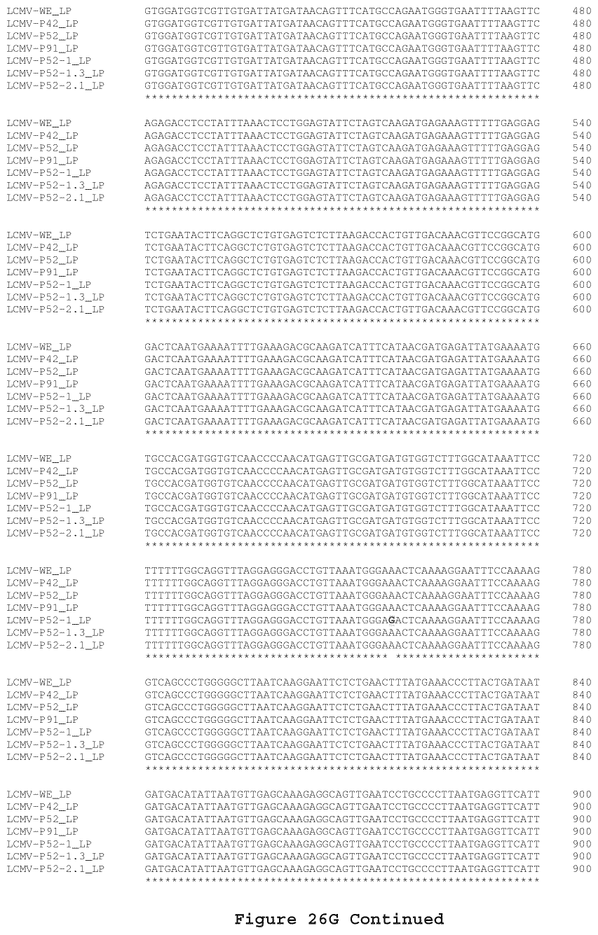

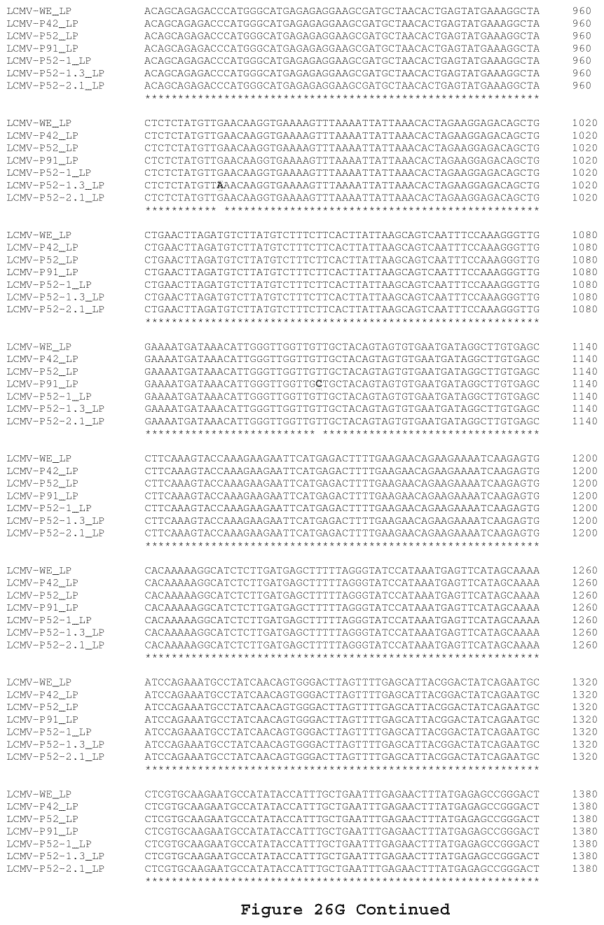

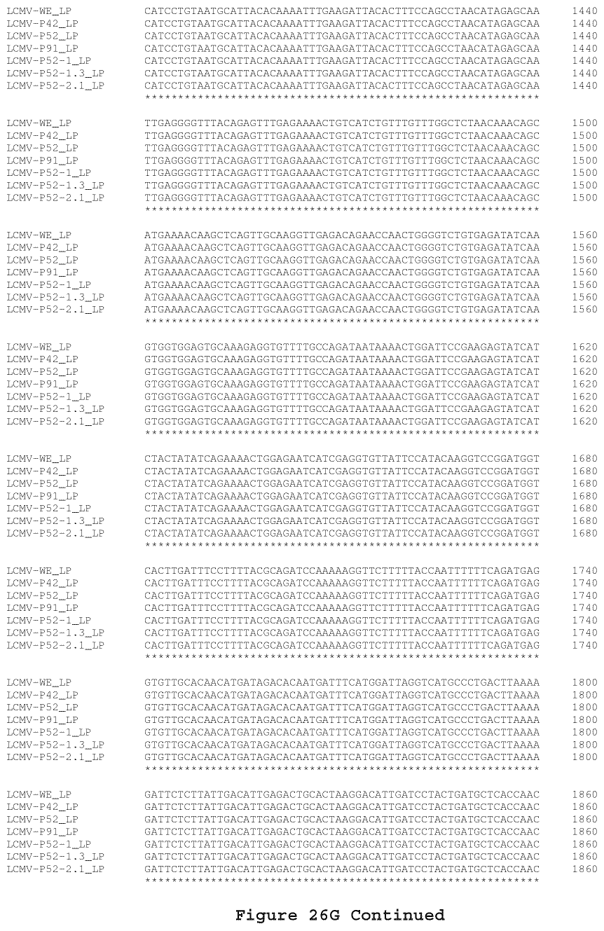

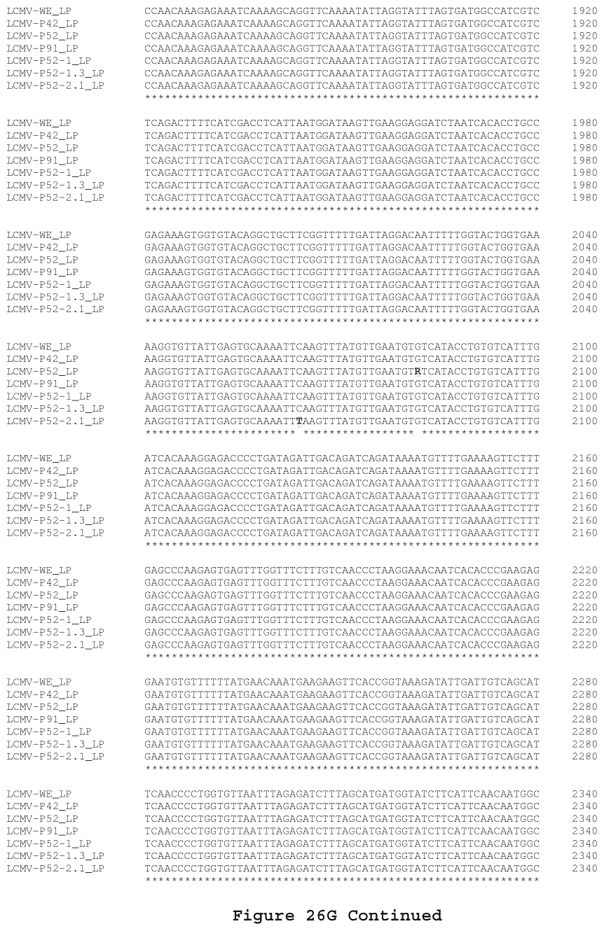

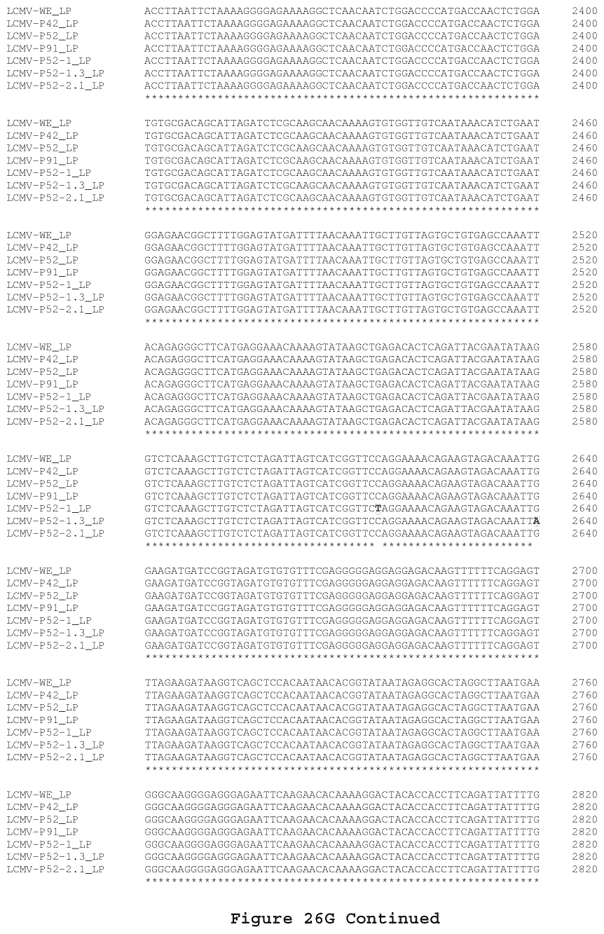

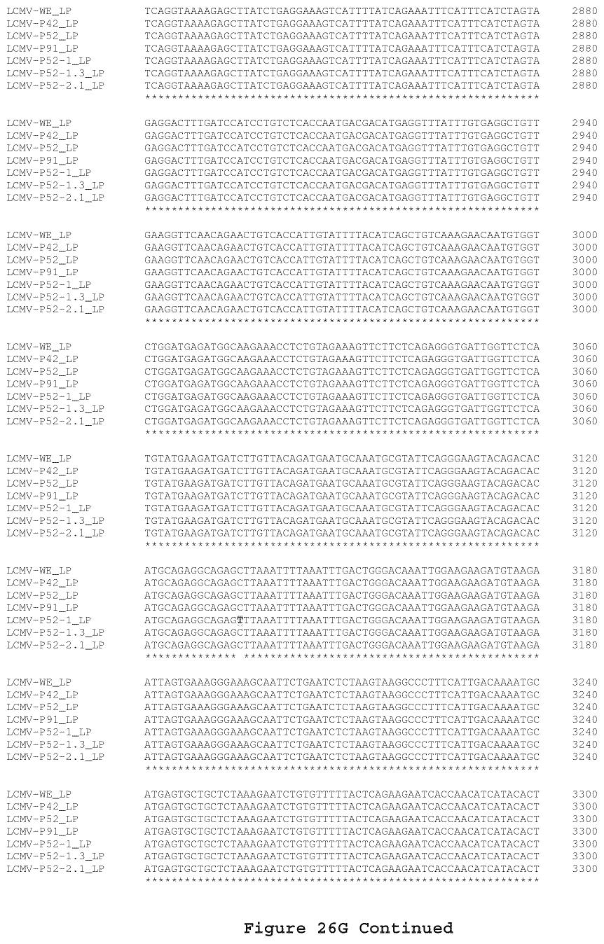

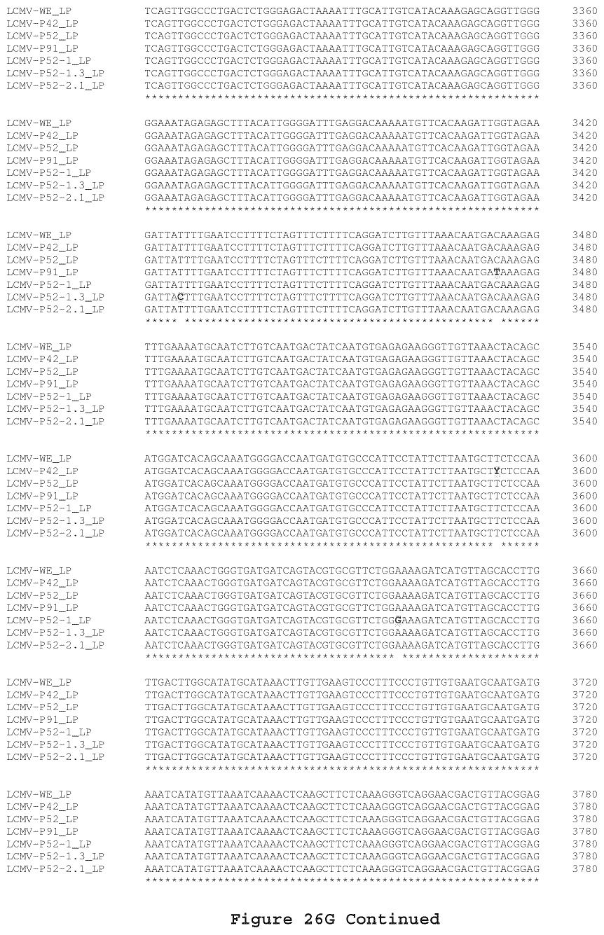

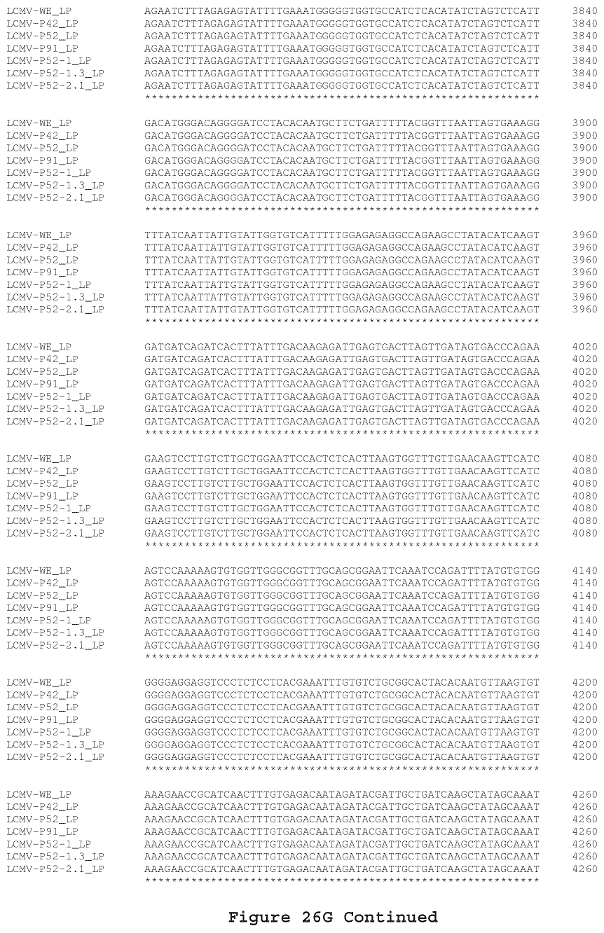

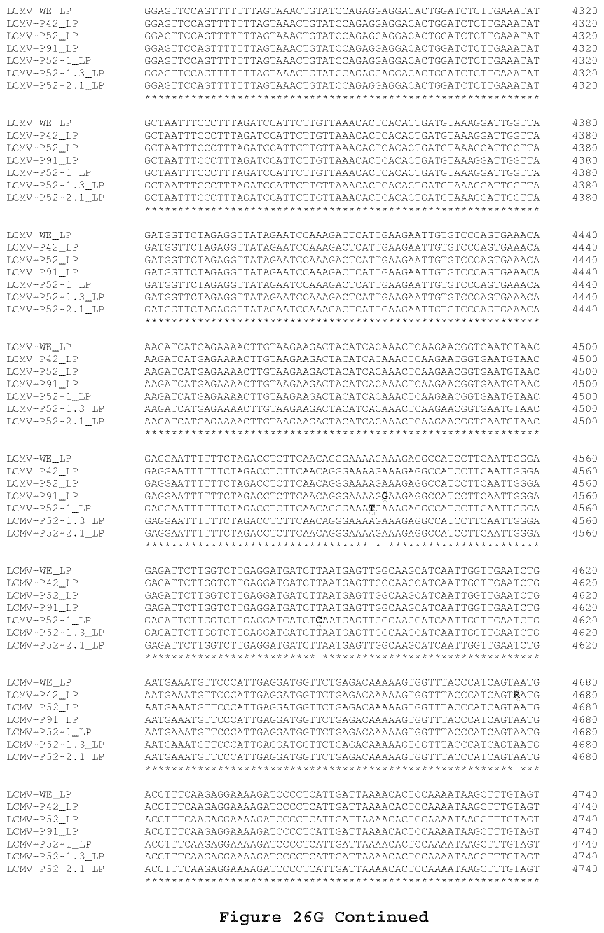

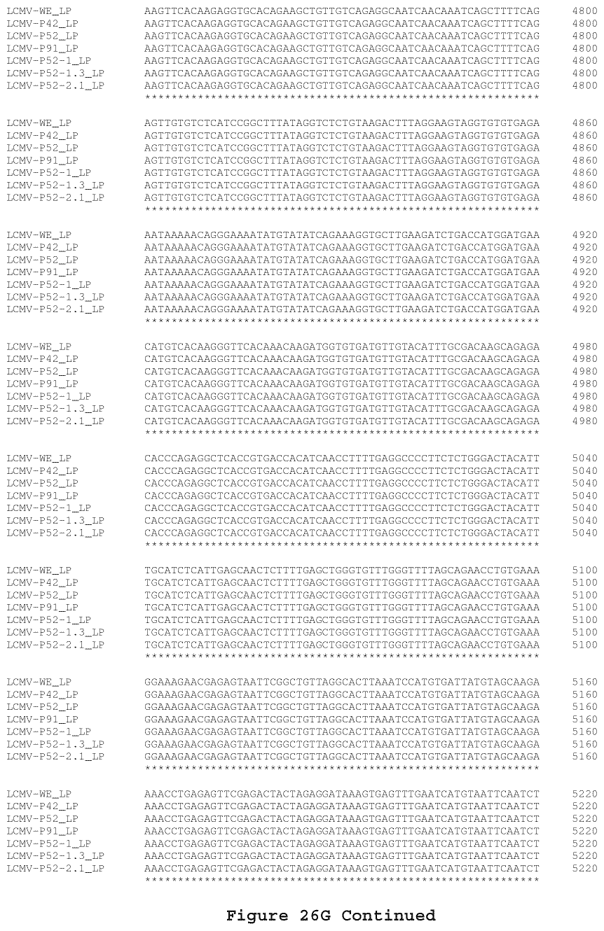

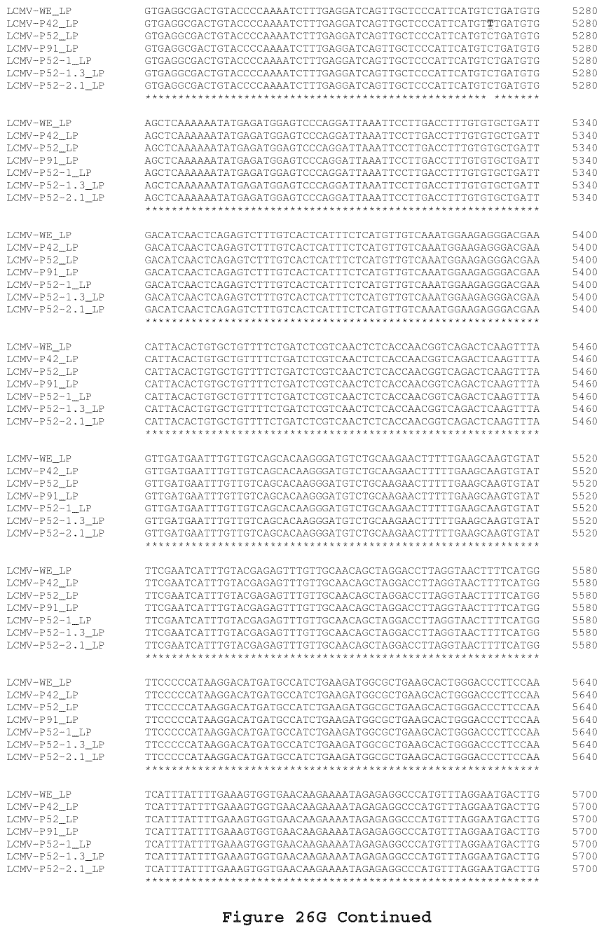

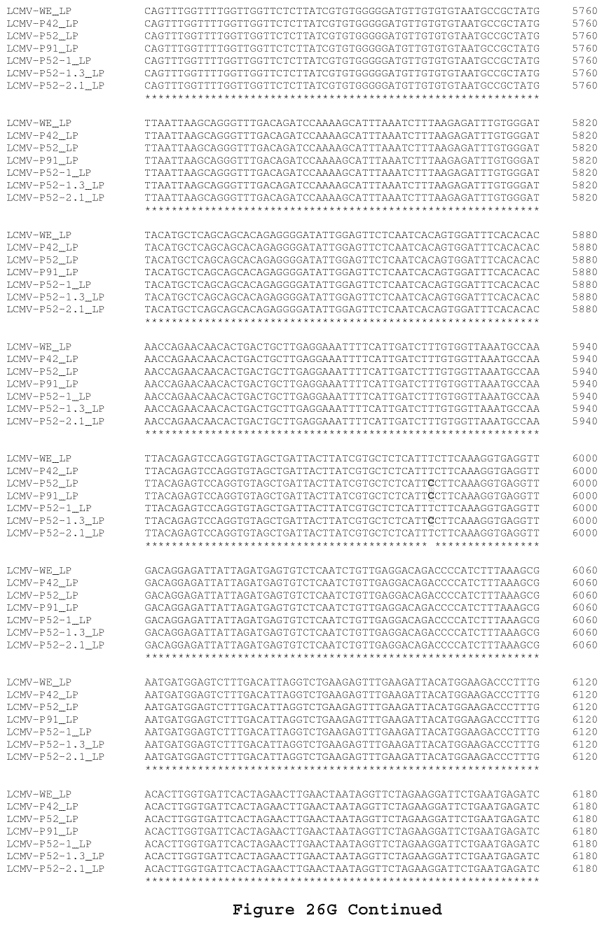

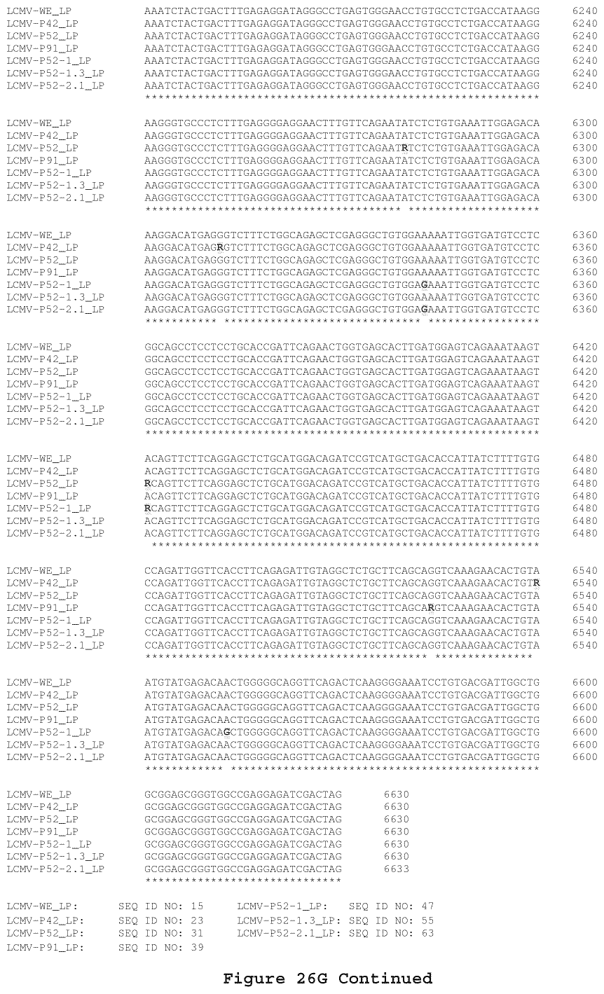

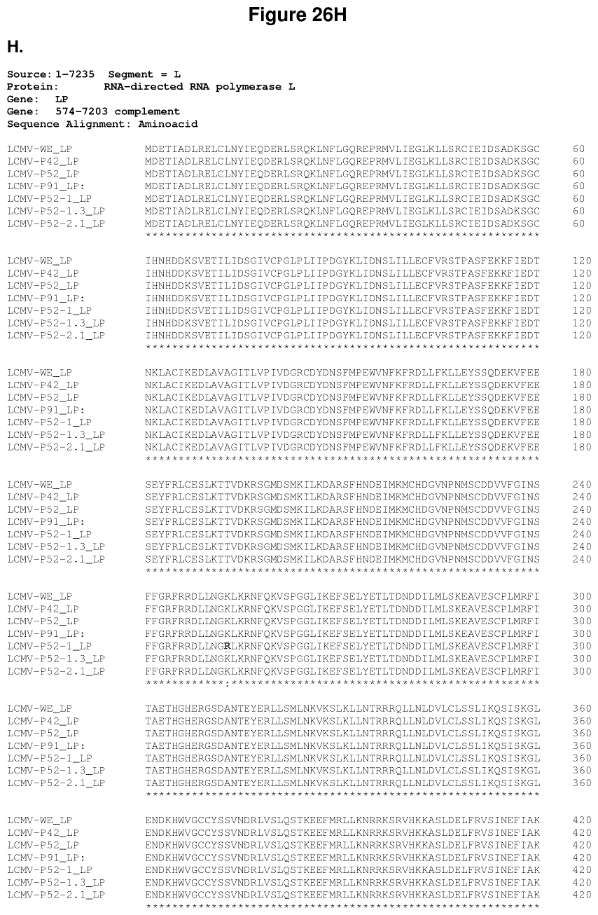

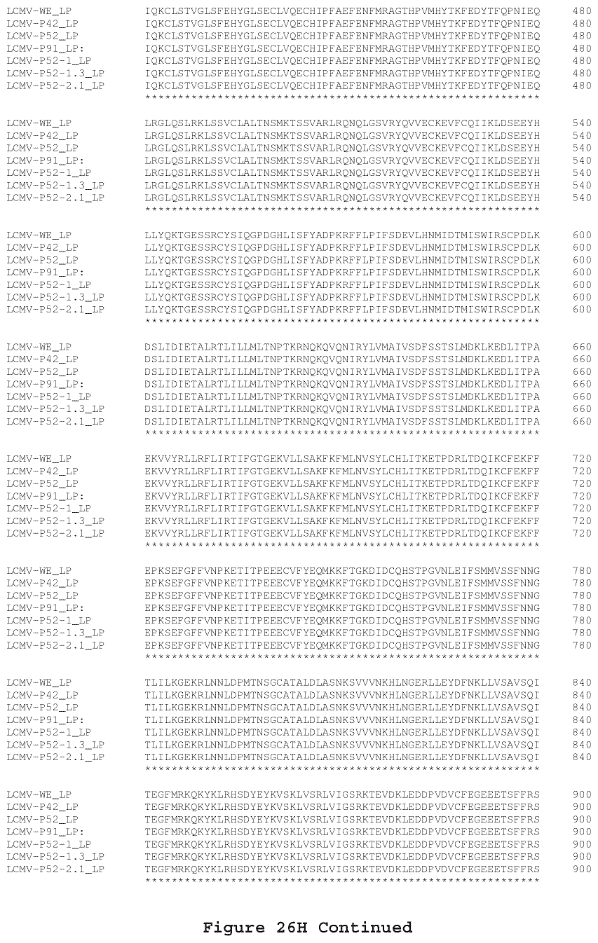

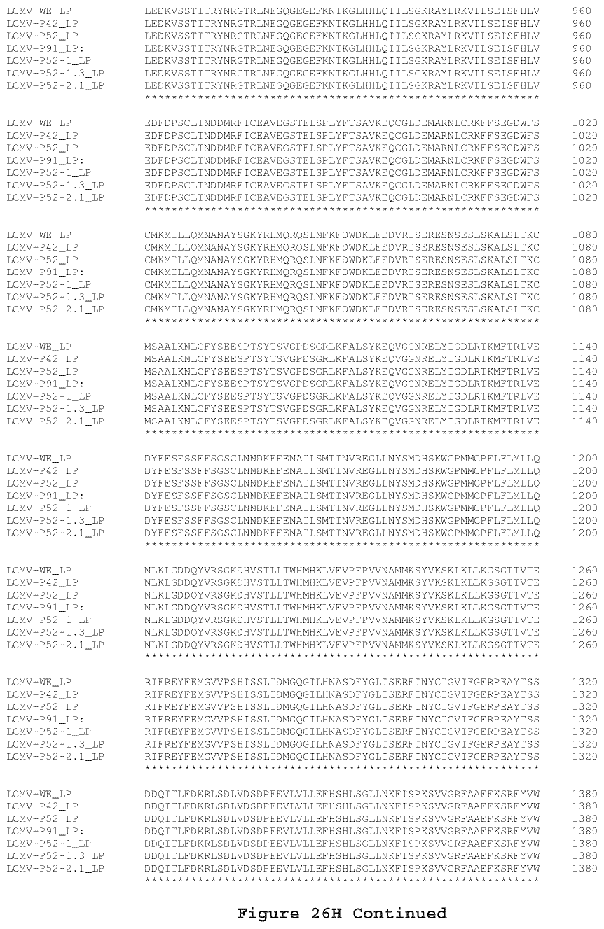

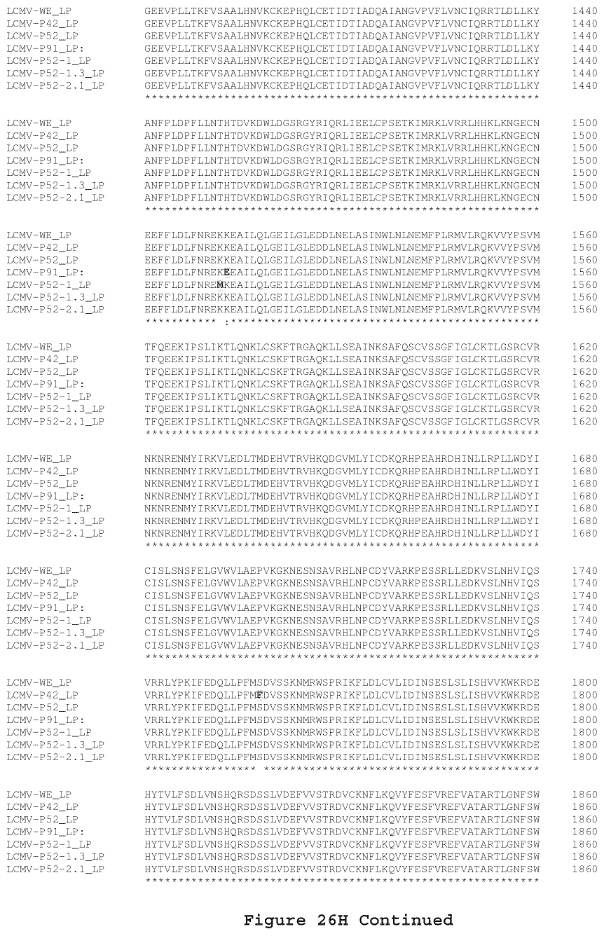

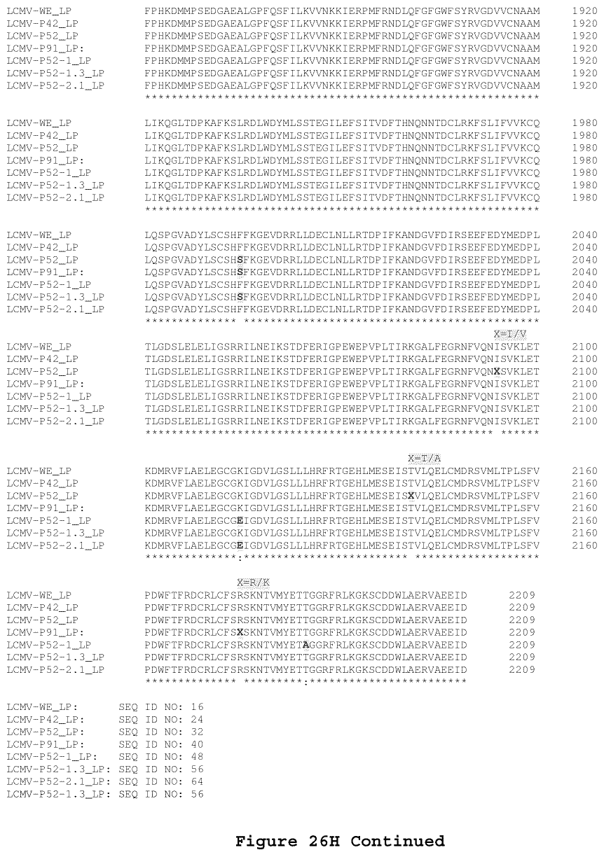

LCMV-WE was passaged 52 times in the tumor cell lines H1975. After 52 passages, the mutations I181M, R185W were detected in the glycoprotein of the new virus. In addition some, most likely irrelevant and/or oligoclonal mutations (quasispecies) were found. This virus is named: “LCMV-P52”. To determine the stability and importance of the mutations I181M and R185W, the supernatant of passage P52 was passaged 39 more times. The virus derived from this passage is named: “LCMV-P91” and still contained the mutations I181M and R185W. The virus LCMV-P52 was subcloned by limiting dilution. This clonal virus is named: “LCMV-P52.1”. The mutations I181M, R185W remained stable. The as irrelevant considered and/or oligoclonal mutations (quasispecies) showed some changes. To determine the role of the single mutations I181M and R185W some earlier passages were analyzed. The passage P29 contained viruses with individual I181M and R185W mutations. Viruses from passage P29 were subcloned by limiting dilution. One subclone with the singular mutation R185W was named: “LCMV-P52-1.3”; one subclone with the singular mutation I181M was named: “LCMV-P52-2.1”. Nucleic acid and amino acid sequences of LCMV mutants P52, P92, P52-1, P52-1.3, and P52-2.1 are shown in FIG. 25 (C-G). Alignments of nucleic acid and amino acid sequences LCMV-WE and mutants P42, P52, P92, P52-1, P52-1.3, and P52-2.1 are shown in FIG. 26 (A-H).

### 2. Investigations

2.1 Tumor cell lines MC57, C643, H1975 and primary tumor cell cultures (UKE Mel-13a) were infected with LCMV (strain WE, MOI 1). Replication was measured after 24 hours (n=3).

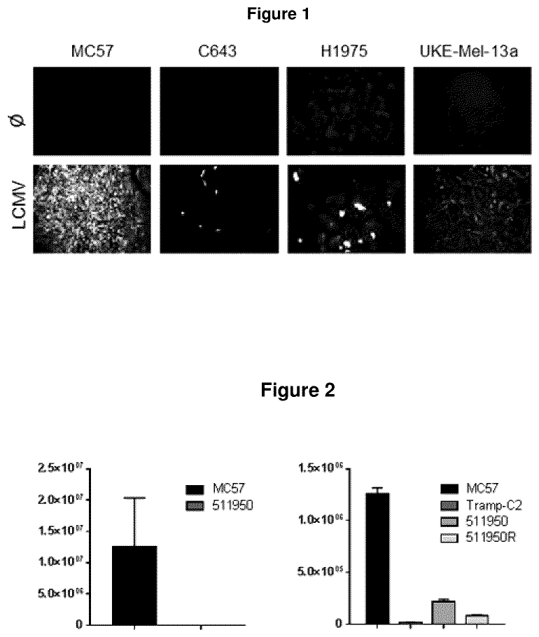

Thereby it was proven that LCMV (strain WE) spread differently. In comparison to the tumor cell line MC57, the spread was reduced in the cell lines C643 and H1975 as well as in primary tumor cell cultures (UKE-Mel-13a). The obtained results are shown graphically in FIG. 1.

In white the LCMV infected cells in the cultures with MC57, C643, H1975 and UKE-Mel-13a can be seen. Ø=without infection, LCMV=LCMV infected.

2.2 Tumor cell line MC57 and primary tumor cell cultures 511950 were infected with LCMV (strain WE, MOI 0,1) (left graph). Tumor cell lines MC57, Tramp-C2 and primary tumor cell cultures (511950 and 511950R) were infected with LCMV (strain WE, MOI 1) (right graph). The number of infectious virus particles was measured after 24 hours in the supernatant.

Thereby it was proven that LCMV (strain WE) proliferate less in comparison to the MC57 tumor cell line in the Tramp-C2 cell line as well as in the primary tumor cell cultures (511950 and 511950R). The results are shown in FIG. 2.

FIG. 2 has the following legend:

Ordinate: Infectious LCMV particles in supernatant (PFU/mL),

Abscissa: Treatment groups; MC57, Tramp-C2, 511950, 511950R.

2.3 LCMV-WE was passaged 42 times in primary tumor cell cultures (UKE-Mel-13a). After 42 passages, a functional mutation was detected in the new virus (LCMV-P42).

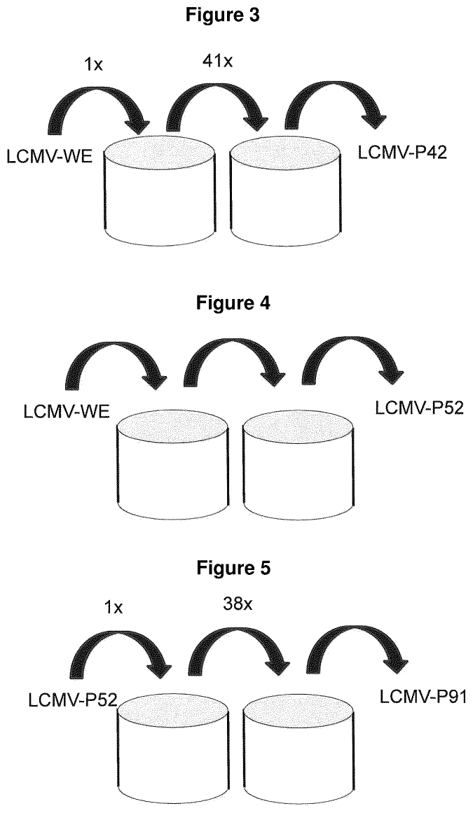

Thereby it could be proven that primary tumor cells are suitable for modifying arenaviruses by passaging. The experimental procedure is shown in FIG. 3.

2.4 LCMV-WE was passaged 52 times in tumor cell lines H1975. After 52 passages, functional mutations were detected in the new virus LCMV-P52.

Thereby, using the example of H1975, it was proven that that tumor cell lines with reduced LCMV replication (compared to cell line MC57) are suitable for altering arenaviruses by passaging. The experimental procedure is illustrated in FIG. 4.

2.5 LCMV-P52 was passaged 39 times in tumor cell lines H1975. After 39 passages further functional mutations were detected in the new virus LCMV-P91.

Thereby it was proven that H1975 cells are capable of altering arenaviruses by passaging. The experimental procedure is shown graphically in FIG. 5.

2.6 Cell lines H1975 were infected with LCMV-WE and LCMV-P52 (MOI 1). Virus was determined after 12 hours in the supernatant (n=6).

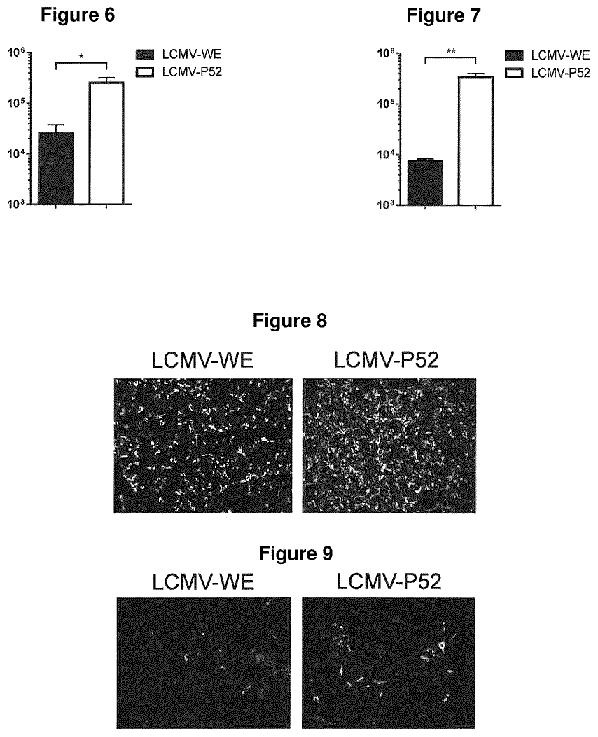

Thereby it was proven that LCMV-P52 replicate better than LCMV-WE in H1975 cells. The results obtained are shown graphically in FIG. 6.

FIG. 6 has the following legend:

Ordinate: Infectious LCMV particles in the supernatant (PFU/mL),

Abscissa: Treatment groups; LCMV-WE or LCMV-P52

2.7 Murine bone marrow-derived dendritic cells were infected with LCMV-WE and LCMV-P52 (MOI 1). Virus was determined after 24 hours in supernatant (n=3).

Thereby it was proven that LCMV-P52 replicate better than LCMV-WE in antigen-presenting cells. The results obtained are shown graphically in FIG. 7.

FIG. 7 has the following legend:

Ordinate: Infectious LCMV particles in the supernatant (PFU/mL),

Abscissa: Treatment groups; LCMV-WE or LCMV-P52

2.8 Tumor cell line H1975 was infected with LCMV-WE or LCMV-P52 (MOI 1). Virus spread was measured by immunofluorescence after 24 hours.

Thereby it was proven that LCMV-P52 can spread better in tumor cell lines compared to LCMV WE using the example of H1975. The results obtained are shown graphically in FIG. 8.

Shown in white are the LCMV infected cells for a culture infected with LCMV-WE or LCMV-P52.

2.9 Primary tumor cells (UKE-Mel-13a) were infected with LCMV-WE or LCMV-P52 (MOI 1). The spread of the viruses was measured by immunofluorescence after 24 hours.

Thereby it was proven that LCMV-P52 can spread better in primary tumor cells compared to LCMV-WE. The obtained results are shown graphically in FIG. 9.

The LCMV infected cells for cultures infected with LCMV-WE or LCMV-P52 are shown in white.

2.10 C57BL/6 mice were infected with LCMV-WE or LCMV-P52 (2×10⁴ PFU intravenously). One day after infection the activation of the innate immune system was determined by measuring IFN-alpha in serum.

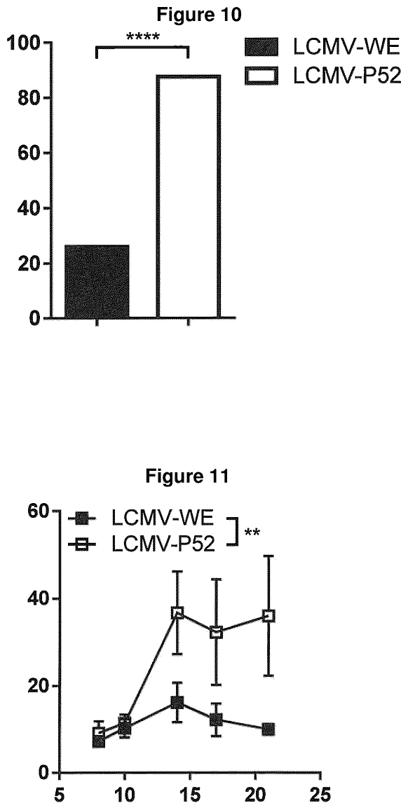

Thereby it was proven that LCMV-P52 causes a stronger innate immune activation than LCMV-WE. The obtained results are shown graphically in FIG. 10.

FIG. 10 has the following legend:

Ordinate: IFN-alpha concentration (ng/mL serum)

Abscissa: Treatment groups; LCMV-WE or LCMV-P52 infected animals.

2.11 C57BL/6 mice were infected with LCMV-WE or LCMV-P52 (2×10⁴ PFU intravenously). Activation of the adaptive immune system was determined by flow cytometry using tetramer (measures virus-specific CD8+ T cells).

Thereby it was proven that LCMV-P52 induces a stronger adaptive immune activation than LCMV-WE. The obtained results are shown graphically in FIG. 11.

FIG. 11 has the following legend:

Ordinate: Virus-specific CD8+ T cells (% of total CD8+ T cells in blood),

Abscissa: Time (days after infection)

2.12 NOD.SCID mice were subcutaneously treated with 5×10⁵ H1975 cells (day −7). The mice were additionally infected with 2×10⁴ PFU LCMV-WE or LCMV-P52 intratumorally (day 0). Tumor growth was analyzed.

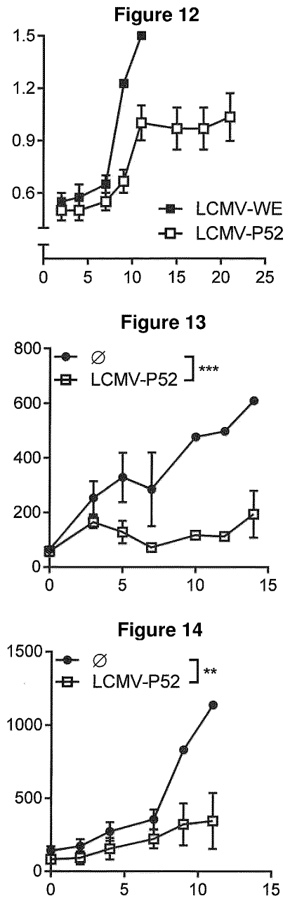

Thereby it was proven that treatment with LCMV-P52 had a stronger anti-tumoral effect than treatment with LCMV-WE. The results obtained are shown graphically in FIG. 12.

FIG. 12 has the following legend:

Ordinate: mean tumor diameter (cm),

Abscissa: Time (days after LCMV treatment)

2.13 C57BL/6 mice were subcutaneously treated with 5×10⁵ MOPC cells (day −7). A group of mice was additionally infected with 2×10⁴ PFU LCMV-P52 intravenously (n=3, day 0). Tumor growth was analyzed.

Thereby it was proven that treatment with LCMV-P52 had a strong anti-tumoral effect. The results are shown graphically in FIG. 13.

FIG. 13 has the following legend:

Ordinate: tumor volume (mm³),

Abscissa: Time (days after LCMV treatment)

2.14 C57BL/6 mice were subcutaneously treated with 5×10⁵ CMT167 cells (day −7). One group of mice was additionally infected with 2×10⁴ PFU LCMV-P52 intravenously (n=3, day 0). Tumor growth was analyzed.

Thereby it was proven that treatment with LCMV-P52 had a strong anti-tumoral effect. The results are shown in FIG. 14.

FIG. 14 has the following legend:

Ordinate: tumor volume (mm³),

Abscissa: Time (days after LCMV treatment)

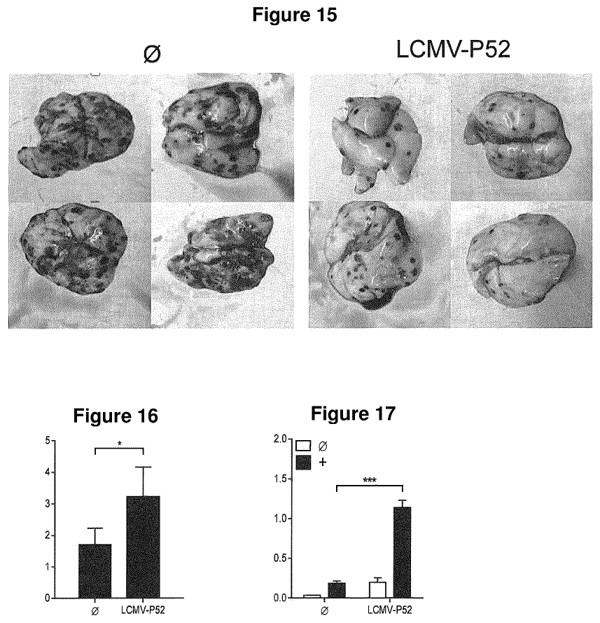

Thereby it was proven that treatment with LCMV-P52 had a strong anti-tumoral effect. The results obtained are shown graphically in FIG. 15.

In black tumor cells in the lung are shown; Ø=without infection

2.15 C57BL/6 mice were treated intravenously with 5×10⁵ B16F10-OVA cells (day −7). Tumor-specific CD8⁺ T cells isolated from the spleen of an OT-1 mouse were transferred (day −4). A group of animals were additionally treated with LCMV-P52 intravenously (2×10⁴ PFU, n=4). On day 9 the lungs were removed and photographed.

Ø=Control animals, 4 lungs each; LCMV-P52=treated with LCMV-P52, 4 lungs each.

2.16 C57BL/6 mice were treated intravenously with 5×10⁵ B16F10-OVA cells (day −7). Tumor-specific CD8⁺ T cells isolated from the spleen of an OT-1 mouse were transferred (day −4). One group of animals were additionally treated with LCMV-P52 intravenously (2×10⁴ PFU, n=4). On day 3, the frequency of tumor-specific CD8⁺ T cells in the blood was determined.

Thereby it was proven that treatment with LCMV-P52 increases the expansion of tumor-specific CD8+ T cells. The results obtained are shown graphically in FIG. 16.

FIG. 16 has the following legend:

Ordinate: Frequency of tumor-specific CD8+ T cells in the blood (% of total CD8+ T cells),

Abscissa: Treatment groups Ø=without infection LCMV-P52=treated with LCMV-P52

2.17 C57BL/6 mice were intravenously treated with 5×10⁵ B16F10-OVA cells (day −7). Tumor-specific CD8⁺ T cells isolated from the spleen of an OT-1 mouse were transferred (day −4). One group of animals were additionally treated with LCMV-P52 intravenously (2×10⁴ PFU, n=4). On day 9, the function of tumor-specific CD8⁺ T cells in the spleen was determined by in vitro restimulation.

Thereby it was proven that treatment with LCMV-P52 increases the function of tumor-specific CD8+ T cells. The obtained results are shown graphically in FIG. 17.

FIG. 17 has the following legend:

Ordinate: Frequency of IFN-gamma-producing tumor-specific CD8+ T cells (% of total CD8+ T cells),

Abscissa: Treatment groups; Ø: Without LCMV-P52 treatment; LCMV-P52: Treatment with LCMV-P52; Legend: −: Without antigen; +: Restimulation with antigen (SIINFEKL peptide).

2.18 C57BL6 mice were subcutaneously treated with 5×10⁵ B16F10-OVA cells (day −7). One group of animals was not further treated (n=3). One group of animals was treated with the inhibitor LCL-161 (oral 50 mg/kg body weight) twice a week from day 0. One group was treated intratumorally with LCMV-WE on day 0 (2×10⁴ PFU, n=4). One group was treated with LCL-161 and LCMV. Tumor growth was analyzed.

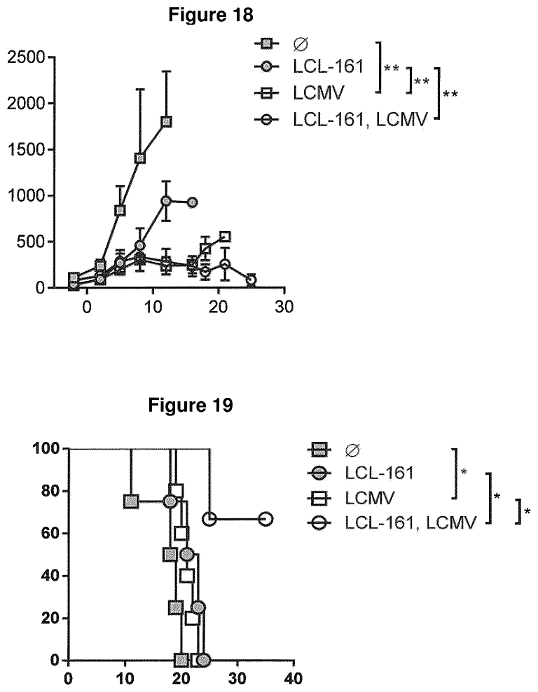

Thereby it was proven that treatment with LCMV-WE has a synergistic effect with LCL-161. The obtained results are shown graphically in FIG. 18.

FIG. 18 has the following legend:

Ordinate: Tumor volume (mm³),

Abscissa: Time (days after LCMV administration)

2.19 C57BL6 mice were subcutaneously treated with 5×10⁵ B16F10-OVA cells (day −7). One group of animals was not further treated (n=3). One group of animals was treated with the inhibitor LCL-161 (oral 50 mg/kg body weight) twice a week from day 0. One group was treated intratumorally with LCMV-WE on day 0 (2×10⁴ PFU, n=4). One group was treated with LCL-161 and LCMV. Survival of the animals was analyzed.

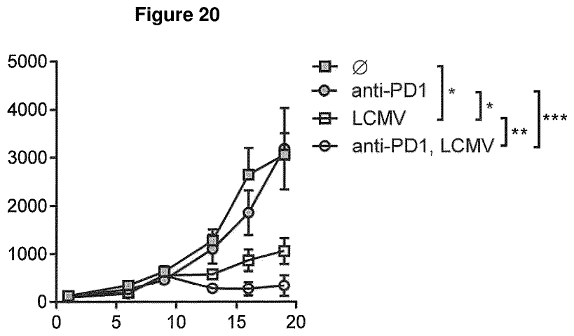

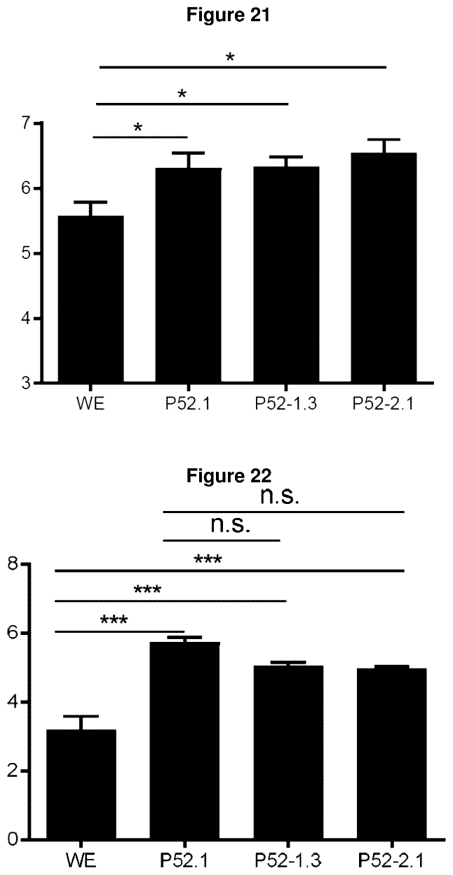

It could be shown that the treatment with LCMV-WE has a synergistic effect with LCL-161. The results obtained are shown graphically in FIG. 19.

FIG. 19 has the following legend:

Ordinate: survival of animals (%),

Abscissa: Time (days after LCMV administration)

2.20 C57BL/6 mice were subcutaneously treated with 5×10⁵ B16F10-OVA cells (day −9). One group of animals was not further treated (n=3). One group was treated intratumorally with LCMV-P52 on day 0 (2×10⁴ PFU, n=6-8). A group of animals was treated with the checkpoint blocker anti-PD-1 (200 μg. intraperitoneal) on days 1, 5 and 8. One group was treated with checkpoint blocker anti-PD-1 and LCMV-P52. Tumor growth was analyzed.

Thereby it was proven that the treatment with LCMV-P52 has a synergistic effect with checkpoint blockers (e.g. anti-PD-1). The results obtained are shown graphically in FIG. 20.

FIG. 20 has the following legend:

Ordinate: tumor volume (mm³),

Abscissa: Time (days after LCMV treatment)

The amino acid and nucleic acid sequences mentioned in the present description correspond to the amino acid and nucleic acid sequences disclosed in the following sequence listing.

2.21 10⁵ H1975 cells were seeded in 24 well plates. After 24 hours cells were infected with the viruses LCMV-WE, LCMV-P52.1 (I181M, R185W), LCMV-P52-1.3 (R185W) and LCMV-P52-2.1 (I181M) with a multiplicity of infection (MOI) of 0.1. Virus was analyzed in the supernatant after 24 hours. The results are shown in FIG. 21 (mean+SEM, n=6, *p<0.05, t-test).

The data show that the mutations 1181M and R185W increase the viral propagation in tumor cells separately.

FIG. 21 has the following legend:

X-axis: Different viruses

Y-axis: LCMV in the supernatant (log₁₀ PFU/ml)

2.22 2.5×10⁵ HCC1954 cells were seeded in 24 well plates, followed by infection of LCMV-WE, LCMV-P52.1 (I181M, R185W), LCMV-P52-1.3 (R185W) and LCMV-P52-2.1 (I181M) with a multiplicity of infection (MOI) of 0.001. Virus was analyzed in the supernatant after 48 hours. The results are shown in FIG. 22 (error bars show SEM, n=4, *p<0.001, n.s. indicates not significant, one-way ANOVA with an additional Tukey post-test was used).

The data show that either the mutation I181M or the mutation 185W increase viral propagation in HCC1954 tumor cells.

FIG. 22 has the following legend:

X-axis: Different viruses

Y-axis: LCMV in the supernatant (log₁₀ PFU/ml)

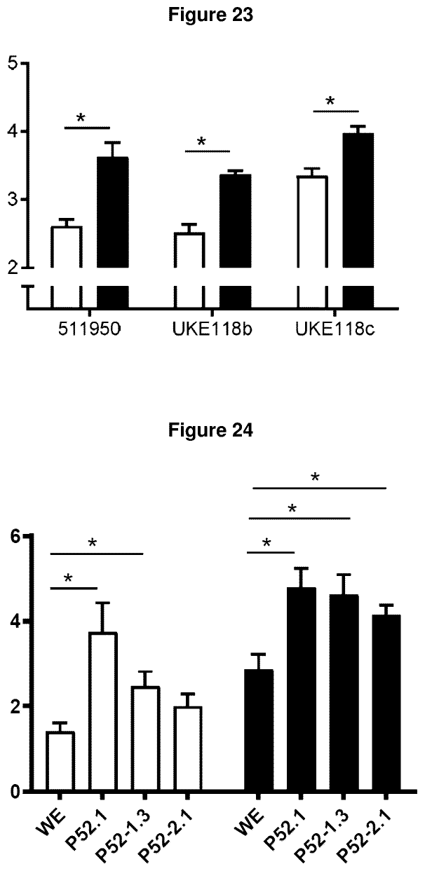

2.23 Murine Pancreatic cancer cells (511950, 4×10⁵ cells), human Melanoma cells (UKE118b, 4×10⁵ cells) or (UKE118c, 4×10⁵ cells) were seeded in 24 well plates, followed by infection with LCMV-WE (white bars) or LCMV-P42 (I181M, black bars) with a multiplicity of infection (MOI) of 0.1. Virus was analyzed in the supernatant after 24 hours. The results are shown in FIG. 23 (mean+SEM, n=3, *p<0.05, t-test).

The data show that the mutation I181M leads to increase viral propagation in tumor cells.

FIG. 23 has the following legend:

X-axis: Different cell types

Y-axis: LCMV in the supernatant (log₁₀ PFU/ml)

2.24 C57BL/6 mice were infected intravenously with 2×10⁴ PFU of either LCMV-WE, LCMV-P52.1 (I181M, R185W), LCMV-P52-1.3 (R185W) and LCMV-P52-2.1 (1181M). On day 8 (white bars) and day 10 (black bars) blood was analyzed for the frequencies of LCMV-GP33-specific CD8⁺ T cells by using tetramers in flow cytometry. The results are shown in FIG. 24 (mean+SEM, n=6-12, pooled from four separate experiments, *p<0.05, t-test).

The data show that the mutations I181M and R185W increase the capacity of LCMV to stimulate specific T cells. The combination of the mutations I181M and R185W might have synergistic properties on early T cell stimulation.

FIG. 24 has the following legend:

X-axis: Different viruses

Y-axis: Frequency of tetramer-GP33-binding CD8⁺ T cells (% of total CD8⁺ T cells).

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A method of treating or preventing a tumor, the method comprising administering to a patient in need thereof a mutant of lymphocytic choriomeningitis virus, wherein the mutant comprises a nucleic acid encoding a glycoprotein comprising a sequence that is at least 95% identical to residues 59-265 of SEQ ID NO: 10 and comprises an Arg 185→Trp amino acid substitution as compared to the wild type glycoprotein sequence set forth in SEQ ID NO: 10.

2. The method of claim 1, wherein the glycoprotein further comprises an Ile181→Met amino acid substitution as compared to the wild type glycoprotein sequence set forth in SEQ ID NO: 10

3. The method of claim 1, wherein the tumor is selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, colorectal carcinoma, hepatocellular carcinoma, testicular carcinoma, colon carcinoma, tumor of the bladder, bladder carcinoma, tumor of the bladder, hepatocellular carcinoma, lung carcinoma, lung carcinoma, endometrial carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, opharyngeal carcinoma, prostate carcinoma, thyroid carcinoma, Cervical cancer, angiosarcoma, chondrosarcoma, Ewing sarcoma, fibrosarcoma, Kaposi sarcoma, liposarcoma, leiomyosarcoma, malignant fibrosis histiocytoma, lymphoma, leukemia, neurogenic sarcoma, osteosarcoma and rhabdomyosarcoma.

4. The method of claim 1, wherein the glycoprotein has at least about 97% sequence identity to the wild type glycoprotein sequence set forth in SEQ ID NO: 10.

5. The method of claim 1, wherein the glycoprotein has at least about 95% sequence identity or is identical to a glycoprotein selected from the group consisting of SEQ ID NOs: 26, 34, and 42.

6. The method of claim 1, wherein the nucleic acid comprises a sequence that has at least about 95% sequence identity or is identical to a sequence selected from the group consisting of SEQ ID NOs: 25, 33, and 41.

7. The method of claim 1, wherein the mutant comprises a nucleic acid encoding a L-protein, wherein said L-protein has at least about 95% sequence identity to the wild type L-protein sequence set forth in SEQ ID NO: 16.

8. The method of claim 1, wherein the mutant comprises a nucleic acid encoding a L-protein, wherein said L-protein has at least about 95% sequence identity or is identical to a sequence selected from the group consisting of SEQ ID NOs: 24, 32, 40, 48, 56, and 64.

9. The method of claim 1, wherein the mutant comprises a nucleic acid encoding a L-protein, wherein said L-protein comprises 1, 2, 3, 4, 5, 6, 7, 8, 9, or 10 of the following amino acid substitutions: Lys 253→Arg, Lys 1512→Met, Lys 1513→Glu, Ser 1758→Phe, Phe 1995→Ser, Ile 2094→Val, Lys 2115→Glu, Thr 2141→Ala, Arg 2175→Lys, or Thr 2185→Ala, as compared to the wild type L-protein sequence set forth in SEQ ID NO: 16.

10. The method of claim 1, wherein the mutant comprises a nucleic acid encoding a L-protein, wherein said L-protein comprises one of the following sets of mutations:
(a) Ser 1758→Phe;
(b) Phe 1995→Ser; optionally Ile 2094→Val; and optionally Thr 2141→Ala;
(c) Lys 1513→Glu; Phe 1995→Ser; and optionally Arg 2175→Lys;
(d) Lys 253→Arg; Lys 1512→Met; Lys 2115→Glu; optionally Thr 2141→Ala; Thr 2185→Ala
(e) Phe 1995→Ser; or
(f) Lys 2115→Glu,
as compared to the wild type L-protein sequence set forth in SEQ ID NO: 16.

11. The method of claim 1, wherein the mutant comprises a nucleic acid encoding an L-protein, wherein said nucleic acid is complementary to a sequence that has at least about 95% sequence identity or that is preferably identical to a sequence selected from the group consisting of SEQ ID NOs: 17, 25, 33, 41, 49, and 57.

12. The mutant claim 1, wherein the mutant comprises a nucleic acid encoding a nucleoprotein, wherein said nucleoprotein has at least about 95% sequence identity or that is identical to a nucleoprotein selected from the group consisting of SEQ ID NOs: 12, 20, 28, 36, 44, 52, and 60.

13. The mutant claim 1, wherein the mutant comprises a nucleic acid encoding a nucleoprotein, wherein said nucleic acid is complementary to a sequence that has at least about 95% sequence identity or that is identical to a sequence selected from the group consisting of SEQ ID NOs: 11, 19, 27, 35, 43, 51, and 59.

14. The method of claim 1, wherein the mutant comprises a nucleic acid encoding a Z-protein, wherein said Z-protein has at least about 95% sequence identity or that is identical to a sequence selected from the group consisting of SEQ ID NOs: 14, 22, 30, 38, 46, 54, and 62.

15. The method of claim 1, wherein the mutant comprises a nucleic acid encoding a Z-protein, wherein said nucleic acid comprises a sequence that has at least about 95% sequence identity or that is identical to a sequence selected from the group consisting of SEQ ID NOs: 13, 21, 29, 37, 45, 53, and 61.

16. The method of claim 1, wherein the method further comprises administering to the subject a checkpoint blocker and/or an apoptosis modulator.

17. A method of treating or preventing a tumor, the method comprising administering to a patient in need thereof a mutant of lymphocytic choriomeningitis virus, wherein the mutant comprises a nucleic acid encoding a glycoprotein comprising a sequence that is at least 99% identical to residues 59-265 of SEQ ID NO: 10 and comprises an Ile181→Met amino acid substitution as compared to the wild type glycoprotein sequence set forth in SEQ ID NO: 10.

18. The method of claim 17, wherein the tumor is selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, colorectal carcinoma, hepatocellular carcinoma, testicular carcinoma, colon carcinoma, tumor of the bladder, bladder carcinoma, tumor of the bladder, hepatocellular carcinoma, lung carcinoma, lung carcinoma, endometrial carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, opharyngeal carcinoma, prostate carcinoma, thyroid carcinoma, Cervical cancer, angiosarcoma, chondrosarcoma, Ewing sarcoma, fibrosarcoma, Kaposi sarcoma, liposarcoma, leiomyosarcoma, malignant fibrosis histiocytoma, lymphoma, leukemia, neurogenic sarcoma, osteosarcoma and rhabdomyosarcoma.

19. A method of treating or preventing a tumor, the method comprising administering to a patient in need thereof a virus comprising a glycoprotein protein comprising:
(i) an amino acid sequence having at least 95% sequence identity to a sequence selected from the group consisting of SEQ ID NO: 26, SEQ ID NO: 34, SEQ ID NO: 42, and SEQ ID NO: 50, wherein the protein comprises Arg 185→Trp and Ile 181→Met amino acid substitutions as compared to the wild type glycoprotein sequence set forth in SEQ ID NO: 10; or
(ii) an amino acid sequence having at least 95% sequence identity to the sequence of SEQ ID NO: 50, wherein the protein comprises an Arg 185→Trp amino acid substitution as compared to the wild type glycoprotein sequence set forth in SEQ ID NO: 10.

20. The method of claim 19, wherein the tumor is selected from the group consisting of anal carcinoma, bronchial carcinoma, lung carcinoma, endometrial carcinoma, gallbladder carcinoma, bladder carcinoma, hepatocellular carcinoma, testicular carcinoma, colon carcinoma, colorectal carcinoma, colorectal carcinoma, hepatocellular carcinoma, testicular carcinoma, colon carcinoma, tumor of the bladder, bladder carcinoma, tumor of the bladder, hepatocellular carcinoma, lung carcinoma, lung carcinoma, endometrial carcinoma, colorectal carcinoma, rectal carcinoma, laryngeal carcinoma, esophageal carcinoma, gastric carcinoma, breast carcinoma, renal carcinoma, ovarian carcinoma, pancreatic carcinoma, pharyngeal carcinoma, opharyngeal carcinoma, prostate carcinoma, thyroid carcinoma, Cervical cancer, angiosarcoma, chondrosarcoma, Ewing sarcoma, fibrosarcoma, Kaposi sarcoma, liposarcoma, leiomyosarcoma, malignant fibrosis histiocytoma, lymphoma, leukemia, neurogenic sarcoma, osteosarcoma and rhabdomyosarcoma.


SEQUENCE LISTING (64 sequences)

SEQ ID NO: 1 - protein, 498 aa, Lymphocytic choriomeningitis virus
  REGION 1..498: note = MISC_FEATURE - Lymphocytic choriomeningitis virus Mutant p52 Glycoprotein; ssrna ambisense-strand viruses; arenaviridae; arenavirus; old world arenaviruses
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAMSQCWTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 2 - DNA, 1497 bp, Lymphocytic choriomeningitis virus
  misc_feature 1..1497: note = MISC_FEATURE - Lymphocytic choriomeningitis virus Mutant p52 Glycoprotein; ssrna ambisense-strand viruses; arenaviridae; arenavirus; old world arenaviruses
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctatgagccagtgttggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 3 - protein, 498 aa, Lymphocytic choriomeningitis virus
  REGION 1..498: note = MISC_FEATURE - Lymphocytic choriomeningitis virus Mutant p42 Glycoprotein; ssrna ambisense-strand viruses; arenaviridae; arenavirus; old world arenaviruses
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAMSQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 4 - DNA, 1497 bp, Lymphocytic choriomeningitis virus
  misc_feature 1..1497: note = MISC_FEATURE - Lymphocytic choriomeningitis virus Mutant p42 Glycoprotein; ssrna ambisense-strand viruses; arenaviridae; arenavirus; old world arenaviruses
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctatgagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 5 - protein, 2209 aa, Lymphocytic choriomeningitis virus
  REGION 1..2209: note = MISC_FEATURE - Lymphocytic choriomeningitis virus Mutant L-Protein; ssrna ambisense-strand viruses; arenaviridae; arenavirus; old world arenaviruses
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKEEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHSFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGKIGDVLGSLLLHRFRTGEHLMESEISTVLQELCMDRSVMLTPLSFVPDWFTFRDCRLCFSKSKNTVMYETTGGRFRLKGKSCDDWLAERVAEEID

SEQ ID NO: 6 - DNA, 6630 bp, Lymphocytic choriomeningitis virus
  misc_feature 1..6630: note = MISC_FEATURE - Lymphocytic choriomeningitis virus Mutant L-Protein; ssrna ambisense-strand viruses; arenaviridae; arenavirus; old world arenaviruses
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgctgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgataaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaaggaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcattccttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggaaaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcaagtcaaagaacactgtaatgtatgagacaactgggggcaggttcagactcaaggggaaatcctgtgacgattggctggcggagcgggtggccgaggagatcgactag

SEQ ID NO: 7 - protein, 2209 aa, Lymphocytic choriomeningitis virus
  REGION 1..2209: note = MISC_FEATURE - Lymphocytic choriomeningitis virus Wildtype L-Protein; ssrna ambisense-strand viruses; arenaviridae; arenavirus; old world arenaviruses
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMFDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHFFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGKIGDVLGSLLLHRFRTGEHLMESEISTVLQELCMDRSVMLTPLSFVPDWFTFRDCRLCFSRSKNTVMYETTGGRFRLKGKSCDDWLAERVAEEID

SEQ ID NO: 8 - DNA, 6630 bp, Lymphocytic choriomeningitis virus
  misc_feature 1..6630: note = MISC_FEATURE - Lymphocytic choriomeningitis virus Wildtype L-Protein; ssrna ambisense-strand viruses; arenaviridae; arenavirus; old world arenaviruses
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaagaaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcatttcttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggaaaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcaggtcaaagaacactgtaatgtatgagacaactgggggcaggttcagactcaaggggaaatcctgtgacgattggctggcggagcgggtggccgaggagatcgactag

SEQ ID NO: 9 - DNA, 1497 bp, Lymphocytic choriomeningitis virus
  misc_feature 1..1497: note = strain WE
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 10 - protein, 498 aa, Lymphocytic choriomeningitis virus
  REGION 1..498: note = misc_feature - strain WE
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 11 - DNA, 1677 bp, Lymphocytic choriomeningitis virus
  misc_feature 1..1677: note = strain WE
  source 1..1677: mol_type = genomic DNA
atgtctttgtccaaagaagtcaaaagctttcagtggacacaggcgttgaggagggagttgcagagttttacatcagatgtaaaggctgccgtcatcaaggacgcaaccagtcttctaaatgggttggacttttctgaagtcagcaacgttcagaggatcatgagaaaggaaaggagggatgataaagacttgcagagactcaggagtcttaaccagactgtgcattctcttgttgatctgaagtctacatcaaagaaaaatgttctgaaagtgggaagacttagtgcagaggaattgatgacccttgcagctgatcttgagaagctgaaggccaaaattatgagaactgagaggcctcaagcttctggagtctacatgggaaatttgacagcacaacaacttgatcaaagatcccaaatactgcaaatggttgggatgagaagacctcagcagggtgcaagtggtgtagtaagggtttgggatgtgaaggactcatcacttctgaacaatcagttcggcacaatgccaagcctgacaatggcttgcatggcaaaacagtcacagaccccactcaatgatgttgtgcaggcactcacagaccttggcttactttacacagtcaaatacccgaatctcagtgatcttgaaaggctaaaggataaacacccagttctgggggtcattactgaacagcaatctagtatcaatatctctggttataatttcagtcttggtgcagctgtgaaagcgggggcagctctgctagatggagggaacatgctggaatctatcttgatcaaaccgagcaacagtgaggatctcctaaaagcagtcctcggggccaagaagaaactcaacatgtttgtctcagatcaagttggagatagaaatccctatgaaaacatcctttataaagtctgtctttcaggtgaaggatggccatacatagcctgtagaacgtcagttgtggggagagcatgggagaacacaacaattgatctcacaaatgaaaaacttgttgccaactcatctaggccagtgcctggagcagcaggcccacctcaggtgggcttgagttacagtcagacaatgctgttgaaagacttgatgggagggattgatcccaatgctcccacatggattgacattgagggcaggttcaatgatccagtggagatagcaatattccaaccacaaaatgggcaattcatacatttttacagggaacctacggaccagaagcaattcaagcaggactcaaagtattcacacggcatggatcttgctgatctcttcaatgcacagcctgggctgacctcatcagttataggtgctctcccacaagggatggttttgagctgtcaaggttctgatgacatcagaaagcttctggactcacaaaatagaagggacataaaactcattgatgttgagatgaccaaggaggcctcaagagaatatgaagataaagtgtgggacaaatatggctggctatgcaaaatgcacactggggtagtgagagacaaaaagaagaaagagatcaccccacactgtgcactcatggactgcatcatttttgagagtgcttccaaggcaagactccctgatctaaaaaccgttcacaacatcctgccacatgatttaatcttcagaggacccaatgttgtgacactctaa

SEQ ID NO: 12 - protein, 558 aa, Lymphocytic choriomeningitis virus
  REGION 1..558: note = misc_feature - strain WE
MSLSKEVKSFQWTQALRRELQSFTSDVKAAVIKDATSLLNGLDFSEVSNVQRIMRKERRDDKDLQRLRSLNQTVHSLVDLKSTSKKNVLKVGRLSAEELMTLAADLEKLKAKIMRTERPQASGVYMGNLTAQQLDQRSQILQMVGMRRPQQGASGVVRVWDVKDSSLLNNQFGTMPSLTMACMAKQSQTPLNDVVQALTDLGLLYTVKYPNLSDLERLKDKHPVLGVITEQQSSINISGYNFSLGAAVKAGAALLDGGNMLESILIKPSNSEDLLKAVLGAKKKLNMFVSDQVGDRNPYENILYKVCLSGEGWPYIACRTSVVGRAWENTTIDLTNEKLVANSSRPVPGAAGPPQVGLSYSQTMLLKDLMGGIDPNAPTWIDIEGRFNDPVEIAIFQPQNGQFIHFYREPTDQKQFKQDSKYSHGMDLADLFNAQPGLTSSVIGALPQGMVLSCQGSDDIRKLLDSQNRRDIKLIDVEMTKEASREYEDKVWDKYGWLCKMHTGVVRDKKKKEITPHCALMDCIIFESASKARLPDLKTVHNILPHDLIFRGPNVVTL

SEQ ID NO: 13 - DNA, 273 bp, Lymphocytic choriomeningitis virus
  misc_feature 1..273: note = strain WE
  source 1..273: mol_type = genomic DNA
atgggccaaggcaagtccaaagaagaaagggacaccagcaatacaggcagagcagagcttttgccagacaccacctatcttggtcctctaaattgtaaatcatgttggcagaaatttgacagcttggttagatgccatgaccactatctttgcagacactgtctgaatctcctgctgtcagtttccgacagatgtcctctctgtaagtatccactgccaaccaaactgaaggtgtcaacagtcccaagctccccacctccctatgaggagtga

SEQ ID NO: 14 - protein, 90 aa, Lymphocytic choriomeningitis virus
  REGION 1..90: note = misc_feature - strain WE
MGQGKSKEERDTSNTGRAELLPDTTYLGPLNCKSCWQKFDSLVRCHDHYLCRHCLNLLLSVSDRCPLCKYPLPTKLKVSTVPSSPPPYEE

SEQ ID NO: 15 - DNA, 6630 bp, Lymphocytic choriomeningitis virus; identical to SEQ ID NO: 8
  misc_feature 1..6630: note = strain WE
  source 1..6630: mol_type = genomic DNA

SEQ ID NO: 16 - protein, 2209 aa, Lymphocytic choriomeningitis virus
  REGION 1..2209: note = misc_feature - strain WE
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHFFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGKIGDVLGSLLLHRFRTGEHLMESEISTVLQELCMDRSVMLTPLSFVPDWFTFRDCRLCFSRSKNTVMYETTGGRFRLKGKSCDDWLAERVAEEID

SEQ ID NO: 17 - DNA, 1497 bp, Lymphocytic choriomeningitis virus; identical to SEQ ID NO: 4
  misc_feature 1..1497: note = mutant P42
  source 1..1497: mol_type = genomic DNA

SEQ ID NO: 18 - protein, 498 aa, Lymphocytic choriomeningitis virus; identical to SEQ ID NO: 3
  REGION 1..498: note = misc_feature - mutant P42

SEQ ID NO: 19 - DNA, 1677 bp, Lymphocytic choriomeningitis virus; identical to SEQ ID NO: 11
  misc_feature 1..1677: note = mutant P42
  source 1..1677: mol_type = genomic DNA

SEQ ID NO: 20 - protein, 558 aa, Lymphocytic choriomeningitis virus; identical to SEQ ID NO: 12
  REGION 1..558: note = misc_feature - mutant P42

SEQ ID NO: 21 - DNA, 273 bp, Lymphocytic choriomeningitis virus; identical to SEQ ID NO: 13
  misc_feature 1..273: note = mutant P42
  source 1..273: mol_type = genomic DNA

SEQ ID NO: 22 - protein, 90 aa, Lymphocytic choriomeningitis virus; identical to SEQ ID NO: 14
  REGION 1..90: note = misc_feature - mutant P42

SEQ ID NO: 23 - DNA, 6630 bp, Lymphocytic choriomeningitis virus
  misc_feature 1..6630: note = mutant P42
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgctyctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaagaaagaggccatccttcaattgggagagattcttggtcttgaggatgatcttaatgagttggcaagcatcaattggttgaatctgaatgaaatgttcccattgaggatggttctgagacaaaaagtggtttacccatcagtratgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtttgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcatttcttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagrgtctttctggcagagctcgagggctgtggaaaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcaggtcaaagaacactgtratgtatgagacaactgggggcaggttcagactcaaggggaaatcctgtgacgattggctggcggagcgggtggccgaggagatcgactag

SEQ ID NO: 24 - protein, 2209 aa, Lymphocytic choriomeningitis virus; identical to SEQ ID NO: 7
  REGION 1..2209: note = misc_feature - mutant P42

SEQ ID NO: 25 - DNA, 1497 bp, Lymphocytic choriomeningitis virus; identical to SEQ ID NO: 2
  misc_feature 1..1497: note = mutant P52
  source 1..1497: mol_type = genomic DNA

SEQ ID NO: 26 - protein, 498 aa, Lymphocytic choriomeningitis virus; identical to SEQ ID NO: 1
  REGION 1..498: note = misc_feature - mutant P52

SEQ ID NO: 27 - DNA, 1677 bp, Lymphocytic choriomeningitis virus
  misc_feature 1..1677: note = mutant P52
  source 1..1677: mol_type = genomic DNA
atgtctttgtccaaagaagtcaaaagctttcagtggacacaggcgttgaggagggagttgcagagttttacatcagatgtaaaggctgccgtcatcaaggacgcaaccagtcttctaaatgggttggacttttctgaagtcagcaacgttcagaggatcatgagaaaggaaaggagggatgataaagacttgcagagactcaggagtcttaaccagactgtgcattctcttgttgatctgaagtctacatcaaagaaaaatgttctgaaagtgggaagacttagtgcagaggaattgatgacccttgcagctgatcttgagaagctgaaggccaaaattatgagaactgagaggcctcaagcttctggagtctacatgggaaatttgacagcacaacaacttgatcaaagatcccaaatactgcaaatggttgggatgagaagacctcagcagggtgcaagtggtgtagtaagggtttgggatgtgaaggactcatcacttctgaacaatcagttcggcacaatgccaagcctgacaatggcttgcatggcaaaacagtcacagaccccactcaatgatgttgtgcaggcactcacagaccttggcttactttacacagtcaaatacccgaatctcagtgatcttgaaaggctaaaggataaacacccagttctgggggtcattactgaacagcaatctagtatcaatatctctggttataatttcagtcttggtgcagctgtgaaagcgggggcagctctgctagatggagggaacatgctggaatctatcttgatcaaaccgagcaacagtgaggatctcctaaaagcagtcctcggggccaagaagaaactcaacatgtttgtctcagatcaagttggagatagaaatccctatgaaaacatcctttataaagtctgtctttcaggtgaaggatggccatacatagcctgtagaacgtcagttgtggggagagcatgggagaacacaacaattgatctcacaaatgaaaaacttgttgccaactcatctaggccagtgcctggagcagcaggcccacctcaggtgggcttgagttacagtcagacaatgctgttgaaagacttgatgggagggattgatcccaatgctcccacatggattgacattgagggcaggttcaatgatccagtggagatagcaatattccaaccacaaaatgggcaattcatacatttttacagggaacctacggaccagaagcaattcaagcaggactcaaagtattcacacggcatggatcttgctgatctcttcaatgcacagcctgggctgacctcatcagttataggtgctctcccacaagggatggttttgagctgtcaaggttctgatgacatcagaaagcttctggactcacaaaayagaagggacataaaactcattgatgttgagatgaccaaggaggcctcaagagaatatgaagataaagtgtgggacaaatatggctggctatgcaaaatgcacactggggtagtgagagacaaaaagaagaaagagatcaccccacactgtgcactcatggactgcatcatttttgagagtgcttccaaggcaagactccctgatctaaaaaccgttcacaacatcctgccacatgatttaatcttcagaggacccaatgttgtgacactctaa

SEQ ID NO: 28 - protein, 558 aa, Lymphocytic choriomeningitis virus; identical to SEQ ID NO: 12
  REGION 1..558: note = misc_feature - mutant P52

SEQ ID NO: 29 - DNA, 273 bp, Lymphocytic choriomeningitis virus; identical to SEQ ID NO: 13
  misc_feature 1..273: note = mutant P52
  source 1..273: mol_type = genomic DNA

SEQ ID NO: 30 - protein, 90 aa, Lymphocytic choriomeningitis virus; identical to SEQ ID NO: 14
  REGION 1..90: note = misc_feature - mutant P52

SEQ ID NO: 31 - DNA, 6630 bp, Lymphocytic choriomeningitis virus
  misc_feature 1..6630: note = mutant P52
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtrtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaagaaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcattccttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatrtctctgtgaaattggagacaaaggacatgagggtctttctggcagagctcgagggctgtggaaaaattggtgatgtcctcggcagcctcctcctgcaccgattcagaactggtgagcacttgatggagtcagaaataagtrcagttcttcaggagctctgcatggacagatccgtcatgctgacaccattatcttttgtgccagattggttcaccttcagagattgtaggctctgcttcagcaggtcaaagaacactgtaatgtatgagacaactgggggcaggttcagactcaaggggaaatcctgtgacgattggctggcggagcgggtggccgaggagatcgactag

SEQ ID NO: 32 - protein, 2209 aa, Lymphocytic choriomeningitis virus
  REGION 1..2209: note = misc_feature - mutant P52
  VARIANT 2094: note = Xaa can be Ile or Val
  VARIANT 2141: note = Xaa can be Thr or Ala
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHSFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNXSVKLETKDMRVFLAELEGCGKIGDVLGSLLLHRFRTGEHLMESEISXVLQELCMDRSVMLTPLSFVPDWFTFRDCRLCFSRSKNTVMYETTGGRFRLKGKSCDDWLAERVAEEID

SEQ ID NO: 33 - DNA, 1497 bp, Lymphocytic choriomeningitis virus; identical to SEQ ID NO: 2
  misc_feature 1..1497: note = mutant P91
  source 1..1497: mol_type = genomic DNA

SEQ ID NO: 34 - protein, 498 aa, Lymphocytic choriomeningitis virus; identical to SEQ ID NO: 1
  REGION 1..498: note = misc_feature - mutant P91

SEQ ID NO: 35 - DNA, 1677 bp, Lymphocytic choriomeningitis virus; identical to SEQ ID NO: 11
  misc_feature 1..1677: note = mutant P91
  source 1..1677: mol_type = genomic DNA

SEQ ID NO: 36 - protein, 558 aa, Lymphocytic choriomeningitis virus; identical to SEQ ID NO: 12
  REGION 1..558: note = misc_feature - mutant P91

SEQ ID NO: 37 - DNA, 273 bp, Lymphocytic choriomeningitis virus; identical to SEQ ID NO: 13
  misc_feature 1..273: note = mutant P91
  source 1..273: mol_type = genomic DNA

SEQ ID NO: 38 - protein, 90 aa, Lymphocytic choriomeningitis virus; identical to SEQ ID NO: 14
  REGION 1..90: note = misc_feature - mutant P91

SEQ ID NO: 39 - DNA, 6630 bp, Lymphocytic choriomeningitis virus
  misc_feature 1..6630: note = mutant P91
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgctgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgataaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaaggaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcattccttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggaaaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcargtcaaagaacactgtaatgtatgagacaactgggggcaggttcagactcaaggggaaatcctgtgacgattggctggcggagcgggtggccgaggagatcgactag

SEQ ID NO: 40 - protein, 2209 aa, Lymphocytic choriomeningitis virus
  REGION 1..2209: note = misc_feature - mutant P91
  VARIANT 2175: note = Xaa can be Arg or Lys
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKEEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHSFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGKIGDVLGSLLLHRFRTGEHLMESEISTVLQELCMDRSVMLTPLSFVPDWFTFRDCRLCFSXSKNTVMYETTGGRFRLKGKSCDDWLAERVAEEID

SEQ ID NO: 41 - DNA, 1497 bp, Lymphocytic choriomeningitis virus
  misc_feature 1..1497: note = mutant P52-1
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggcctcaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctatgagccagtgttggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 42 - protein, 498 aa, Lymphocytic choriomeningitis virus; identical to SEQ ID NO: 1
  REGION 1..498: note = misc_feature - mutant P52-1

SEQ ID NO: 43 - DNA, 1677 bp, Lymphocytic choriomeningitis virus; identical to SEQ ID NO: 11
  misc_feature 1..1677: note = mutant P52-1
  source 1..1677: mol_type = genomic DNA

SEQ ID NO: 44 - protein, 558 aa, Lymphocytic choriomeningitis virus; identical to SEQ ID NO: 12
  REGION 1..558: note = misc_feature - mutant P52-1

SEQ ID NO: 45 - DNA, 273 bp, Lymphocytic choriomeningitis virus; identical to SEQ ID NO: 13
  misc_feature 1..273: note = mutant P52-1
  source 1..273: mol_type = genomic DNA

SEQ ID NO: 46 - protein, 90 aa, Lymphocytic choriomeningitis virus; identical to SEQ ID NO: 14
  REGION 1..90: note = misc_feature - mutant P52-1

SEQ ID NO: 47 - DNA, 6630 bp, Lymphocytic choriomeningitis virus
  misc_feature 1..6630: note = mutant P52-1
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggagactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttctaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagtttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctgggaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaatgaaagaggccatccttcaattgggagagattcttggtcttgaggatgatctcaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcatttcttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggagaaattggtgatgtcctcggcagcctcctcctgcaccgattcagaactggtgagcacttgatggagtcagaaataagtrcagttcttcaggagctctgcatggacagatccgtcatgctgacaccattatcttttgtgccagattggttcaccttcagagattgtaggctctgcttcagcaggtcaaagaacactgtaatgtatgagacagctgggggcaggttcagactcaaggggaaatcctgtgacgattggctggcggagcgggtggccgaggagatcgactag

SEQ ID NO: 48 - protein, 2209 aa, Lymphocytic choriomeningitis virus
  REGION 1..2209: note = misc_feature - mutant P52-1
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGRLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREMKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHFFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGEIGDVLGSLLLHRFRTGEHLMESEISTVLQELCMDRSVMLTPLSFVPDWFTFRDCRLCFSRSKNTVMYETAGGRFRLKGKSCDDWLAERVAEEID

SEQ ID NO: 49 - DNA, 1497 bp, Lymphocytic choriomeningitis virus
  misc_feature 1..1497: note = mutant P52-1.3
  source 1..1497: mol_type = genomic DNA
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgttggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaacacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 50 - protein, 498 aa, Lymphocytic choriomeningitis virus
  REGION 1..498: note = misc_feature - mutant P52-1.3
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCWTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 51 - DNA, 1677 bp, Lymphocytic choriomeningitis virus
  misc_feature 1..1677: note = mutant P52-1.3
  source 1..1677: mol_type = genomic DNA
atgtctttgtccaaagaagtcaaaagctttcagtggacacaggcgttgaggagggagttgcagagttttacatcagatgtaaaggctgccgtcatcaaggacgcaaccagtcttctaaatgggttggacttttctgaagtcagcaacgttcagaggatcatgagaaaggaaaggagggatgataaagacttgcagagactcaggagtcttaaccagactgtgcattctcttgttgatctgaagtctacatcaaagaaaaatgttctgaaagtgggaagacttagtgcagaggaattgatgacccttgcagctgatctcgagaagctgaaggccaaaattatgagaactgagaggcctcaagcttctggagtctacatgggaaatttgacagcacaacaacttgatcaaagatcccaaatactgcaaatggttgggatgagaagacctcagcagggtgcaagtggtgtagtaagggtttgggatgtgaaggactcatcacttctgaacaatcagttcggcacaatgccaagcctgacaatggcttgcatggcaaaacagtcacagaccccactcaatgatgttgtgcaggcactcacagaccttggcttactttacacagtcaaatacccgaatctcagtgatcttgaaaggctaaaggataaacacccagttctgggggtcattactgaacagcaatctagtatcaatatctctggttataatttcagtcttggtgcagctgtgaaagcgggggcagctctgctagatggagggaacatgctggaatctatcttgatcaaaccgagcaacagtgaggatctcctaaaagcagtcctcggggccaagaagaaactcaacatgtttgtctcagatcaagttggagatagaaatccctatgaaaacatcctttataaagtctgtctttcaggtgaaggatggccatacatagcctgtagaacgtcagttgtggggagagcatgggagaacacaacaattgatctcacaaatgaaaaacttgttgccaactcatctaggccagtgcctggagcagcaggcccacctcaggtgggcttgagttacagtcagacaatgctgttgaaagacttgatgggagggattgatcccaatgctcccacatggattgacattgagggcaggttcaatgatccagtggagatagcaatattccaaccacaaaatgggcaattcatacatttttacagggaacctacggaccagaagcaattcaagcaggactcaaagtattcacacggcatggatcttgctgatctcttcaatgcacagcctgggctgacctcatcagttataggtgctctcccacaagggatggttttgagctgtcaaggttctgatgacatcagaaagcttctggactcacaaaatagaagggacataaaactcattgatgttgagatgaccaaggaggcctcaagagaatatgaagataaagtgtgggacaaatatggctggctatgcaaaatgcacactggggtagtgagagacaaaaagaagaaagagatcaccccacactgtgcactcatggactgcatcatttttgagagtgcttccaaggcaagactccctgatctaaaaaccgttcacaacatcctgccacatgatttaatcttcagaggacccaatgttgtgacactctaa

SEQ ID NO: 52 - protein, 558 aa, Lymphocytic choriomeningitis virus; identical to SEQ ID NO: 12
  REGION 1..558: note = misc_feature - mutant P52-1.3

SEQ ID NO: 53 - DNA, 273 bp, Lymphocytic choriomeningitis virus; identical to SEQ ID NO: 13
  misc_feature 1..273: note = mutant P52-1.3
  source 1..273: mol_type = genomic DNA

SEQ ID NO: 54 - protein, 90 aa, Lymphocytic choriomeningitis virus; identical to SEQ ID NO: 14
  REGION 1..90: note = misc_feature - mutant P52-1.3

SEQ ID NO: 55 - DNA, 6630 bp, Lymphocytic choriomeningitis virus
  misc_feature 1..6630: note = mutant P52-1.3
  source 1..6630: mol_type = genomic DNA
atggatgaaactattgcagatttgagagagttgtgtctaaattacatagaacaggacgagaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttaaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaattcaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattagaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattactttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaagaaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcattccttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggaaaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcaggtcaaagaacactgtaatgtatgagacaactgggggcaggttcagactcaaggggaaatcctgtgacgattggctggcggagcgggtggccgaggagatcgactag

SEQ ID NO: 56 - protein, 2209 aa, Lymphocytic choriomeningitis virus
  REGION 1..2209: note = misc_feature - mutant P52-1.3
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHSFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGKIGDVLGSLLLHRFRTGEHLMESEISTVLQELCMDRSVMLTPLSFVPDWFTFRDCRLCFSRSKNTVMYETTGGRFRLKGKSCDDWLAERVAEEID

SEQ ID NO: 57 - DNA, 1497 bp, Lymphocytic choriomeningitis virus
  misc_feature 1..1497: note = mutant P52-2.1
  source 1..1497: mol_type = genomic DNA
atgggtcagattgtgacaatgtttgaggctttgcctcacatcattgatgaggtcatcaacattgttattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctatgagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 58 - protein, 498 aa, Lymphocytic choriomeningitis virus; identical to SEQ ID NO: 3
  REGION 1..498: note = misc_feature - mutant P52-2.1

SEQ ID NO: 59 - DNA, 1677 bp, Lymphocytic choriomeningitis virus
  misc_feature 1..1677: note = mutant P52-2.1
  source 1..1677: mol_type = genomic DNA
atgtctttgtccaaagaagtcaaaagctttcagtggacacaggcgttgaggagggagttgcagagttttacatcagatgtaaaggctgccgtcatcaaggacgcaaccagtcttctaaatgggttggacttttctgaagtcagcaacgttcagaggatcatgagaaaggaaaggagggatgataaagacttgcagagactcaggagtcttaaccagactgtgcattctcttgttgatctgaagtctacatcaaagaaaaatgttctgaaagtgggaagacttagtgcagaggaattgatgacccttgcagctgatcttgagaagctgaaggccaaaattatgagaactgagaggcctcaagcttctggagtctacatgggaaatttgacagcacaacaacttgatcaaagatcccaaatactgcaaatggttgggatgagaagacctcagcagggtgcaagtggtgtagtaagggtttgggatgtgaaggactcatcacttctgaacaatcagttcggcacaatgccaagcctgacaatggcttgcatggcaaaacagtcacagaccccactcaatgatgttgtgcaggcactcacagaccttggcttactttacacagtcaaatacccgaatctcagtgatcttgaaaggctaaaggataaacacccagttctgggggtcattactgaacagcaatctagtatcaatatctctggttataatttcagtcttggtgcagctgtgaaagcgggggcagctctgctagatggagggaacatgctggaatctatcttgatcaaaccgagcaacagtgaggatctcctaaaagcagtcctcggggccaaaaagaaactcaacatgtttgtctcagatcaagttggagatagaaatccctatgaaaacatcctttataaagtctgtctttcaggtgaaggatggccatacatagcctgtagaacgtcagttgtggggagagcatgggagaacacaacaattgatctcacaaatgaaaaacttgttgccaactcatctaggccagtgcctggagcagcaggcccacctcaggtgggcttgagttacagtcagacaatgctgttgaaagacttgatgggagggattgatcccaatgctcccacatggattgacattgagggcaggttcaatgatccagtggagatagcaatattccaaccacaaaatgggcaattcatacatttttacagggaacctacggaccagaagcaattcaagcaggactcaaagtattcacacggcatggatcttgctgatctcttcaatgcacagcctgggctgacctcatcagttataggtgctctcccacaagggatggttttgagctgtcaaggttctgatgacatcagaaagcttctggactcacaaaatagaagggacataaaactcattgatgttgagatgaccaaggaggcctcaagagaatatgaagataaagtgtgggacaaatatggctggctatgcaaaatgcacactggggtagtgagagacaaaaagaagaaagagatcaccccacactgtgcactcatggactgcatcatttttgagagtgcttccaaggcaagactccctgatctaaaaaccgttcacaacatcctgccacatgatttaatcttcagaggacccaatgttgtgacactctaa

SEQ ID NO: 60 - protein, 558 aa, Lymphocytic choriomeningitis virus; identical to SEQ ID NO: 12
  REGION 1..558: note = misc_feature - mutant P52-2.1

SEQ ID NO: 61 - DNA, 273 bp, Lymphocytic choriomeningitis virus; identical to SEQ ID NO: 13
  misc_feature 1..273: note = mutant P52-2.1
  source 1..273: mol_type = genomic DNA

SEQ ID NO: 62 - protein, 90 aa, Lymphocytic choriomeningitis virus; identical to SEQ ID NO: 14
  REGION 1..90: note = misc_feature - mutant P52-2.1

SEQ ID NO: 63 - DNA, 6630 bp, Lymphocytic choriomeningitis virus
  misc_feature 1..6630: note = mutant P52-2.1
  source 1..6630: mol_type = genomic DNA
atggatgaaactattgcagatttgagagagttgtgtctaaattacatagaacaggacgaaaggctgtcaaggcaaaaactcaacttcctgggacaaagagaacccagaatggtgctaattgagggactcaaattgttatcacgctgtatagagatagacagtgcagacaaaagtggttgcatacacaaccacgatgacaaatctgttgaaacaatcctaatagactctgggattgtgtgtccaggactgccactcatcatccctgatggttataagttgattgacaattcccttattcttcttgaatgttttgttagaagcacaccagctagttttgaaaagaagttcattgaggacaccaacaaactagcatgcatcaaagaagatcttgctgttgcaggcatcacactggttccaatagtggatggtcgttgtgattatgataacagtttcatgccagaatgggtgaattttaagttcagagacctcctatttaaactcctggagtattctagtcaagatgagaaagtttttgaggagtctgaatacttcaggctctgtgagtctcttaagaccactgttgacaaacgttccggcatggactcaatgaaaattttgaaagacgcaagatcatttcataacgatgagattatgaaaatgtgccacgatggtgtcaaccccaacatgagttgcgatgatgtggtctttggcataaattccttttttggcaggtttaggagggacctgttaaatgggaaactcaaaaggaatttccaaaaggtcagccctgggggcttaatcaaggaattctctgaactttatgaaacccttactgataatgatgacatattaatgttgagcaaagaggcagttgaatcctgccccttaatgaggttcattacagcagagacccatgggcatgagagaggaagcgatgctaacactgagtatgaaaggctactctctatgttgaacaaggtgaaaagtttaaaattattaaacactagaaggagacagctgctgaacttagatgtcttatgtctttcttcacttattaagcagtcaatttccaaagggttggaaaatgataaacattgggttggttgttgctacagtagtgtgaatgataggcttgtgagccttcaaagtaccaaagaagaattcatgagacttttgaagaacagaagaaaatcaagagtgcacaaaaaggcatctcttgatgagctttttagggtatccataaatgagttcatagcaaaaatccagaaatgcctatcaacagtgggacttagttttgagcattacggactatcagaatgcctcgtgcaagaatgccatataccatttgctgaatttgagaactttatgagagccgggactcatcctgtaatgcattacacaaaatttgaagattacactttccagcctaacatagagcaattgaggggtttacagagtttgagaaaactgtcatctgtttgtttggctctaacaaacagcatgaaaacaagctcagttgcaaggttgagacagaaccaactggggtctgtgagatatcaagtggtggagtgcaaagaggtgttttgccagataataaaactggattccgaagagtatcatctactatatcagaaaactggagaatcatcgaggtgttattccatacaaggtccggatggtcacttgatttccttttacgcagatccaaaaaggttctttttaccaattttttcagatgaggtgttgcacaacatgatagacacaatgatttcatggattaggtcatgccctgacttaaaagattctcttattgacattgagactgcactaaggacattgatcctactgatgctcaccaacccaacaaagagaaatcaaaagcaggttcaaaatattaggtatttagtgatggccatcgtctcagacttttcatcgacctcattaatggataagttgaaggaggatctaatcacacctgccgagaaagtggtgtacaggctgcttcggtttttgattaggacaatttttggtactggtgaaaaggtgttattgagtgcaaaatttaagtttatgttgaatgtgtcatacctgtgtcatttgatcacaaaggagacccctgatagattgacagatcagataaaatgttttgaaaagttctttgagcccaagagtgagtttggtttctttgtcaaccctaaggaaacaatcacacccgaagaggaatgtgttttttatgaacaaatgaagaagttcaccggtaaagatattgattgtcagcattcaacccctggtgttaatttagagatctttagcatgatggtatcttcattcaacaatggcaccttaattctaaaaggggagaaaaggctcaacaatctggaccccatgaccaactctggatgtgcgacagcattagatctcgcaagcaacaaaagtgtggttgtcaataaacatctgaatggagaacggcttttggagtatgattttaacaaattgcttgttagtgctgtgagccaaattacagagggcttcatgaggaaacaaaagtataagctgagacactcagattacgaatataaggtctcaaagcttgtctctagattagtcatcggttccaggaaaacagaagtagacaaattggaagatgatccggtagatgtgtgtttcgagggggaggaggagacaagttttttcaggagtttagaagataaggtcagctccacaataacacggtataatagaggcactaggcttaatgaagggcaaggggagggagaattcaagaacacaaaaggactacaccaccttcagattattttgtcaggtaaaagagcttatctgaggaaagtcattttatcagaaatttcatttcatctagtagaggactttgatccatcctgtctcaccaatgacgacatgaggtttatttgtgaggctgttgaaggttcaacagaactgtcaccattgtattttacatcagctgtcaaagaacaatgtggtctggatgagatggcaagaaacctctgtagaaagttcttctcagagggtgattggttctcatgtatgaagatgatcttgttacagatgaatgcaaatgcgtattcagggaagtacagacacatgcagaggcagagcttaaattttaaatttgactgggacaaattggaagaagatgtaagaattagtgaaagggaaagcaattctgaatctctaagtaaggccctttcattgacaaaatgcatgagtgctgctctaaagaatctgtgtttttactcagaagaatcaccaacatcatacacttcagttggccctgactctgggagactaaaatttgcattgtcatacaaagagcaggttgggggaaatagagagctttacattggggatttgaggacaaaaatgttcacaagattggtagaagattattttgaatccttttctagtttcttttcaggatcttgtttaaacaatgacaaagagtttgaaaatgcaatcttgtcaatgactatcaatgtgagagaagggttgttaaactacagcatggatcacagcaaatggggaccaatgatgtgcccattcctattcttaatgcttctccaaaatctcaaactgggtgatgatcagtacgtgcgttctggaaaagatcatgttagcaccttgttgacttggcatatgcataaacttgttgaagtccctttccctgttgtgaatgcaatgatgaaatcatatgttaaatcaaaactcaagcttctcaaagggtcaggaacgactgttacggagagaatctttagagagtattttgaaatgggggtggtgccatctcacatatctagtctcattgacatgggacaggggatcctacacaatgcttctgatttttacggtttaattagtgaaaggtttatcaattattgtattggtgtcatttttggagagaggccagaagcctatacatcaagtgatgatcagatcactttatttgacaagagattgagtgacttagttgatagtgacccagaagaagtccttgtcttgctggaattccactctcacttaagtggtttgttgaacaagttcatcagtccaaaaagtgtggttgggcggtttgcagcggaattcaaatccagattttatgtgtggggggaggaggtccctctcctcacgaaatttgtgtctgcggcactacacaatgttaagtgtaaagaaccgcatcaactttgtgagacaatagatacgattgctgatcaagctatagcaaatggagttccagtttttttagtaaactgtatccagaggaggacactggatctcttgaaatatgctaatttccctttagatccattcttgttaaacactcacactgatgtaaaggattggttagatggttctagaggttatagaatccaaagactcattgaagaattgtgtcccagtgaaacaaagatcatgagaaaacttgtaagaagactacatcacaaactcaagaacggtgaatgtaacgaggaattttttctagacctcttcaacagggaaaagaaagaggccatccttcaattgggagagattcttggtcttgaggatgatcttaatgagttggcaagcatcaattggttgaatctgaatgaaatgttcccattgaggatggttctgagacaaaaagtggtttacccatcagtaatgacctttcaagaggaaaagatcccctcattgattaaaacactccaaaataagctttgtagtaagttcacaagaggtgcacagaagctgttgtcagaggcaatcaacaaatcagcttttcagagttgtgtctcatccggctttataggtctctgtaagactttaggaagtaggtgtgtgagaaataaaaacagggaaaatatgtatatcagaaaggtgcttgaagatctgaccatggatgaacatgtcacaagggttcacaaacaagatggtgtgatgttgtacatttgcgacaagcagagacacccagaggctcaccgtgaccacatcaaccttttgaggccccttctctgggactacatttgcatctcattgagcaactcttttgagctgggtgtttgggttttagcagaacctgtgaaaggaaagaacgagagtaattcggctgttaggcacttaaatccatgtgattatgtagcaagaaaacctgagagttcgagactactagaggataaagtgagtttgaatcatgtaattcaatctgtgaggcgactgtaccccaaaatctttgaggatcagttgctcccattcatgtctgatgtgagctcaaaaaatatgagatggagtcccaggattaaattccttgacctttgtgtgctgattgacatcaactcagagtctttgtcactcatttctcatgttgtcaaatggaagagggacgaacattacactgtgctgttttctgatctcgtcaactctcaccaacggtcagactcaagtttagttgatgaatttgttgtcagcacaagggatgtctgcaagaactttttgaagcaagtgtatttcgaatcatttgtacgagagtttgttgcaacagctaggaccttaggtaacttttcatggttcccccataaggacatgatgccatctgaagatggcgctgaagcactgggacccttccaatcatttattttgaaagtggtgaacaagaaaatagagaggcccatgtttaggaatgacttgcagtttggttttggttggttctcttatcgtgtgggggatgttgtgtgtaatgccgctatgttaattaagcagggtttgacagatccaaaagcatttaaatctttaagagatttgtgggattacatgctcagcagcacagaggggatattggagttctcaatcacagtggatttcacacacaaccagaacaacactgactgcttgaggaaattttcattgatctttgtggttaaatgccaattacagagtccaggtgtagctgattacttatcgtgctctcatttcttcaaaggtgaggttgacaggagattattagatgagtgtctcaatctgttgaggacagaccccatctttaaagcgaatgatggagtctttgacattaggtctgaagagtttgaagattacatggaagaccctttgacacttggtgattcactagaacttgaactaataggttctagaaggattctgaatgagatcaaatctactgactttgagaggatagggcctgagtgggaacctgtgcctctgaccataaggaagggtgccctctttgaggggaggaactttgttcagaatatctctgtgaaattggagacaaaggacatgagggtctttctggcagagctcgagggctgtggagaaattggtgatgtcctcggcagcctcctcctgcaccgattcagaactggtgagcacttgatggagtcagaaataagtacagttcttcaggagctctgcatggacagatccgtcatgctgacaccattatcttttgtgccagattggttcaccttcagagattgtaggctctgcttcagcaggtcaaagaacactgtaatgtatgagacaactgggggcaggttcagactcaaggggaaatcctgtgacgattggctggcggagcgggtggccgaggagatcgactag

SEQ ID NO: 64 - protein, 2209 aa, Lymphocytic choriomeningitis virus
  REGION 1..2209: note = misc_feature - mutant P52-2.1
MDETIADLRELCLNYIEQDERLSRQKLNFLGQREPRMVLIEGLKLLSRCIEIDSADKSGCIHNHDDKSVETILIDSGIVCPGLPLIIPDGYKLIDNSLILLECFVRSTPASFEKKFIEDTNKLACIKEDLAVAGITLVPIVDGRCDYDNSFMPEWVNFKFRDLLFKLLEYSSQDEKVFEESEYFRLCESLKTTVDKRSGMDSMKILKDARSFHNDEIMKMCHDGVNPNMSCDDVVFGINSFFGRFRRDLLNGKLKRNFQKVSPGGLIKEFSELYETLTDNDDILMLSKEAVESCPLMRFITAETHGHERGSDANTEYERLLSMLNKVKSLKLLNTRRRQLLNLDVLCLSSLIKQSISKGLENDKHWVGCCYSSVNDRLVSLQSTKEEFMRLLKNRRKSRVHKKASLDELFRVSINEFIAKIQKCLSTVGLSFEHYGLSECLVQECHIPFAEFENFMRAGTHPVMHYTKFEDYTFQPNIEQLRGLQSLRKLSSVCLALTNSMKTSSVARLRQNQLGSVRYQVVECKEVFCQIIKLDSEEYHLLYQKTGESSRCYSIQGPDGHLISFYADPKRFFLPIFSDEVLHNMIDTMISWIRSCPDLKDSLIDIETALRTLILLMLTNPTKRNQKQVQNIRYLVMAIVSDFSSTSLMDKLKEDLITPAEKVVYRLLRFLIRTIFGTGEKVLLSAKFKFMLNVSYLCHLITKETPDRLTDQIKCFEKFFEPKSEFGFFVNPKETITPEEECVFYEQMKKFTGKDIDCQHSTPGVNLEIFSMMVSSFNNGTLILKGEKRLNNLDPMTNSGCATALDLASNKSVVVNKHLNGERLLEYDFNKLLVSAVSQITEGFMRKQKYKLRHSDYEYKVSKLVSRLVIGSRKTEVDKLEDDPVDVCFEGEEETSFFRSLEDKVSSTITRYNRGTRLNEGQGEGEFKNTKGLHHLQIILSGKRAYLRKVILSEISFHLVEDFDPSCLTNDDMRFICEAVEGSTELSPLYFTSAVKEQCGLDEMARNLCRKFFSEGDWFSCMKMILLQMNANAYSGKYRHMQRQSLNFKFDWDKLEEDVRISERESNSESLSKALSLTKCMSAALKNLCFYSEESPTSYTSVGPDSGRLKFALSYKEQVGGNRELYIGDLRTKMFTRLVEDYFESFSSFFSGSCLNNDKEFENAILSMTINVREGLLNYSMDHSKWGPMMCPFLFLMLLQNLKLGDDQYVRSGKDHVSTLLTWHMHKLVEVPFPVVNAMMKSYVKSKLKLLKGSGTTVTERIFREYFEMGVVPSHISSLIDMGQGILHNASDFYGLISERFINYCIGVIFGERPEAYTSSDDQITLFDKRLSDLVDSDPEEVLVLLEFHSHLSGLLNKFISPKSVVGRFAAEFKSRFYVWGEEVPLLTKFVSAALHNVKCKEPHQLCETIDTIADQAIANGVPVFLVNCIQRRTLDLLKYANFPLDPFLLNTHTDVKDWLDGSRGYRIQRLIEELCPSETKIMRKLVRRLHHKLKNGECNEEFFLDLFNREKKEAILQLGEILGLEDDLNELASINWLNLNEMFPLRMVLRQKVVYPSVMTFQEEKIPSLIKTLQNKLCSKFTRGAQKLLSEAINKSAFQSCVSSGFIGLCKTLGSRCVRNKNRENMYIRKVLEDLTMDEHVTRVHKQDGVMLYICDKQRHPEAHRDHINLLRPLLWDYICISLSNSFELGVWVLAEPVKGKNESNSAVRHLNPCDYVARKPESSRLLEDKVSLNHVIQSVRRLYPKIFEDQLLPFMSDVSSKNMRWSPRIKFLDLCVLIDINSESLSLISHVVKWKRDEHYTVLFSDLVNSHQRSDSSLVDEFVVSTRDVCKNFLKQVYFESFVREFVATARTLGNFSWFPHKDMMPSEDGAEALGPFQSFILKVVNKKIERPMFRNDLQFGFGWFSYRVGDVVCNAAMLIKQGLTDPKAFKSLRDLWDYMLSSTEGILEFSITVDFTHNQNNTDCLRKFSLIFVVKCQLQSPGVADYLSCSHFFKGEVDRRLLDECLNLLRTDPIFKANDGVFDIRSEEFEDYMEDPLTLGDSLELELIGSRRILNEIKSTDFERIGPEWEPVPLTIRKGALFEGRNFVQNISVKLETKDMRVFLAELEGCGEIGDVLGSLLLHRFRTGEHLMESEISTVLQELCMDRSVMLTPLSFVPDWFTFRDCRLCFSRSKNTVMYETTGGRFRLKGKSCDDWLAERVAEEID